(19) United States
(12) Patent Application Publication
(10) Pub. No.: US 2022/0372519 A1
(43) Pub. Date: Nov. 24, 2022

(54) IN VITRO ASSEMBLY OF ANELLOVIRUS CAPSIDS ENCLOSING RNA

(71) Applicant: FLAGSHIP PIONEERING INNOVATIONS V, INC., Cambridge, MA (US)

(72) Inventors: Roger Joseph Hajjar, Lexington, MA (US); Simon Delagrave, Sudbury, MA (US); Kurt Adam Swanson, West Newton, MA (US)

(21) Appl. No.: 17/846,503

(22) Filed: Jun. 22, 2022

Related U.S. Application Data
(63) Continuation of application No. PCT/US2021/064887, filed on Dec. 22, 2021.
(60) Provisional application No. 63/147,064, filed on Feb. 8, 2021.
(60) Provisional application No. 63/130,360, filed on Dec. 23, 2020.

(51) Int. Cl. C12N 15/86

(52) U.S. Cl. CPC C12N 15/86

(57) ABSTRACT

This invention relates generally to compositions for making anellovectors and uses thereof.

### CROSS REFERENCE TO RELATED APPLICATIONS

This application is a continuation of PCT/US2021/064887, filed Dec. 22, 2021, which claims the benefit of U.S. Provisional Application No. 63/130,360, filed Dec. 23, 2020 and U.S. Provisional Application No. 63/147,064, filed Feb. 8, 2021. The contents of the aforesaid applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on, Jun. 22, 2022, is named V2057-701720FT_SL.txt and is 286,722 bytes in size.

### BACKGROUND

There is an ongoing need to develop compositions and methods for making suitable vectors to deliver therapeutic effectors to patients.

### SUMMARY

The present disclosure provides compositions and methods for producing an anellovector (e.g., a synthetic anellovector) that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). While naturally occurring Anellovirus has a DNA genome, the present disclosure provides anellovectors with a genetic element that comprises RNA.

An anellovector (e.g., produced using a composition or method as described herein) generally comprises a genetic element (e.g., a genetic element comprising or encoding an effector, e.g., an exogenous or endogenous effector, e.g., a therapeutic effector) encapsulated in a proteinaceous exterior (e.g., a proteinaceous exterior comprising an Anellovirus capsid protein, e.g., an Anellovirus ORF1 protein or a polypeptide encoded by an Anellovirus ORF1 nucleic acid, e.g., as described herein), which is capable of introducing the genetic element into a cell (e.g., a mammalian cell, e.g., a human cell). The genetic element may comprise RNA. In some embodiments, the anellovector is an infectious vehicle or particle comprising a proteinaceous exterior comprising a polypeptide encoded by an Anellovirus ORF1 nucleic acid (e.g., an ORF1 nucleic acid of Alphatorquevirus, Betatorquevirus, or Gammatorquevirus, e.g., an ORF1 of Alphatorquevirus clade 1, Alphatorquevirus clade 2, Alphatorquevirus clade 3, Alphatorquevirus clade 4, Alphatorquevirus clade 5, Alphatorquevirus clade 6, or Alphatorquevirus clade 7, e.g., as described herein). The genetic element of an anellovector of the present disclosure is typically a circular and/or single-stranded RNA molecule (e.g., circular and single stranded) having a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some embodiments, the genetic element of an anellovector is produced using a baculovirus, nucleic acid construct (e.g., a bacmid and/or donor vector), insect cell, and/or animal cell line, e.g., as described herein.

In some instances, the genetic element comprises or encodes an effector (e.g., comprises a nucleic acid effector, such as a non-coding RNA, or encodes a polypeptide effector, e.g., a protein), e.g., which can be expressed in a target cell. In some embodiments, the effector is a therapeutic agent or a therapeutic effector, e.g., as described herein. In some embodiments, the effector is an endogenous effector or an exogenous effector, e.g., exogenous to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell (e.g., as described in Examples 3 and 4 of PCT/US19/65995). In another example, the anellovector can deliver and express an effector, e.g., an exogenous protein, in vivo (e.g., as described in Examples 19 and 28 of PCT/US19/65995). Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject.

In some embodiments, the compositions and methods described herein can be used to produce the genetic element of a synthetic anellovector, e.g., in a host cell. A synthetic anellovector has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors include an exogenous genetic element enclosed within a proteinaceous exterior, which can be used for delivering the genetic element, or an effector (e.g., an exogenous effector or an endogenous effector) encoded therein (e.g., a polypeptide or nucleic acid effector), into eukaryotic (e.g., human) cells. In embodiments, the anellovector does not cause a detectable and/or an unwanted immune or inflammatory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector may be substantially non-immunogenic to the target cell, tissue or subject.

In some embodiments, the compositions and methods described herein can be used to produce the genetic element of an anellovector comprising: (i) a proteinaceous exterior comprising an ORF1 molecule; and (ii) a genetic element comprising RNA; wherein the genetic element is enclosed within the proteinaceous exterior. In some embodiments, the genetic element consists of at least 10%, 20%, 25%, 30%, 40%, 50%, 60%, 70%, 75%, 80%, 85%, 90%, 95%, 96%, 97%, 98%, 99%, or 100% RNA. In some embodiments, the genetic element does not comprise DNA. In some embodiments, the genetic element does not comprise ssDNA. Alternatively, in some embodiments, the genetic element comprises a DNA region. In some embodiments, a DNA or RNA molecule described herein comprises one or more modified nucleotides (e.g., a base modification, sugar modification, or backbone modification). In some embodiments, the genetic element is a single-stranded. In some embodiments, the genetic element comprises a double stranded region. In some embodiments the genetic element is a linear polypeptide. Alternatively, in some embodiments, the genetic element is a circular polynucleotide. In some embodiments, the nucleic acid sequence is codon-optimized, e.g., for expression in an insect cell. In some embodiments, at least 50%, 60%, 70%, 80%, 90%, 95%, 96%, 97%, 98%, 99%, or 100% of the codons in the nucleic acid sequence are codon-optimized, e.g., for expression in an insect cell. In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell. In some embodiments, the genetic element is about 10-20, 20-30, 30-40, 50-60 60-70, 70-80, 80-90, 90-100, 100-125, 125-150, 150-200, 200-300, 300-400, 400-500, 500-600, 600-700, 700-800, 800-900, 900-1000, 1000-1500, 1500-2000, 2000-2500, 2500-3000, 3000-3500, 3500-4000, or 4000-4500 nucleotides in length. In some embodiments, the genetic element is at least about 10, 20, 30, 40, 50, 60, 70, 80, 90, 100, 125, 150, 175, 200, 250, 300, 400, 500, 600, 700, 800, 900, 1000, 1500, 2000, 2500, 3000, 3500, 4000, or 4500 nucleotides in length.

In some embodiments, the compositions and methods described herein can be used to produce the genetic element of an infectious (e.g., to a human cell) Annellovector, vehicle, or particle comprising a capsid (e.g., a capsid comprising an Anellovirus ORF, e.g., ORF1, polypeptide) encapsulating a genetic element comprising a protein binding sequence that binds to the capsid and a heterologous (to the Anellovirus) sequence encoding a therapeutic effector. In embodiments, the Anellovector is capable of delivering the genetic element into a mammalian, e.g., human, cell.

In an aspect, the invention features a method of making an anellovector by in vitro assembly. In some embodiments, a method of making an anellovector comprises: (a) providing a mixture comprising: (i) a genetic element comprising RNA, and (ii) an ORF1 molecule; and (b) incubating the mixture under conditions suitable for enclosing the genetic element within a proteinacous comprising the ORF1 molecule, thereby making an anellovector; optionally wherein the mixture is not comprised in a cell. In some embodiments, a method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, a method further comprises, prior to the providing of (a), purifying the ORF1 molecule expressed by the host cell.

In some embodiments, anellovectors, as described herein, can be used as effective delivery vehicles for introducing an agent, such as an effector described herein, to a target cell, e.g., a target cell in a subject to be treated therapeutically or prophylactically.

In an aspect, the invention features a pharmaceutical composition comprising an anellovector (e.g., a synthetic anellovector) as described herein. In some embodiments, the pharmaceutical composition further comprises a pharmaceutically acceptable carrier or excipient. In embodiments, the pharmaceutical composition comprises a unit dose comprising about 10⁵-10¹⁴ (e.g., about 10⁶-10¹³, 10⁷-10¹², 10⁸-10¹¹, or 10⁹-10¹⁰) genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In some embodiments, the payload is a nucleic acid. In some embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

Additional features of any of the aforesaid anellovectors, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### ENUMERATED EMBODIMENTS

1. An anellovector comprising:
a) proteinaceous exterior comprising an ORF1 molecule;
b) a genetic element comprising RNA,

wherein the genetic element is enclosed within the proteinaceous exterior.

2. The anellovector of embodiment 1, wherein the genetic element consists of at least 10%, 20%, 25%, 30%, 40%, 50%, 60%, 70%, 75%, 80%, 85%, 90%, 95%, 96%, 97%, 98%, 99%, or 100% RNA.

3. The anellovector of embodiment 1 or 2, wherein the RNA comprises one or more chemical modifications.

4. The anellovector of any of the preceding embodiments, wherein the genetic element consists of or consists essentially of RNA.

5. The anellovector of any of the preceding embodiments, wherein the anellovector does not comprise DNA.

6. The anellovector of any of the preceding embodiments, wherein the anellovector does not comprise ssDNA.

7. The anellovector of any of the preceding embodiments, wherein the genetic element comprises a DNA region.

8. The anellovector of any of the preceding embodiments, wherein all nucleotides of the DNA region are chemically modified.

9. The anellovector of embodiment 7 or 8, wherein the DNA region is covalently linked to the RNA of the genetic element.

10. The anellovector of any of the preceding embodiments, wherein at least a portion of the DNA region hybridizes to at least a portion of the RNA of the genetic element.

11. The anellovector of any of the preceding embodiments, wherein the DNA region is 10-20, 20-30, 30-40, 40-50, 50-60, 60-70, 70-80, 80-90, 90-100, 100-150, 150-200, 200-300, 300-400, 400-500, 500-600, 600-700, 700-800, 800-900, or 900-1000 nucleotides in length.

12. The anellovector of any of the preceding embodiments, wherein the genetic element is single stranded.

13. The anellovector of any of the preceding embodiments, wherein the genetic element comprises a double stranded region (e.g., a region of RNA pairing with RNA or a DNA pairing with RNA).

14. The anellovector of any of the preceding embodiments, wherein the genetic element is linear.

15. The anellovector of any of the preceding embodiments, wherein the genetic element is circular.

16. The anellovector of any of the preceding embodiments, wherein the genetic element comprises a first region and a second region that can hybridize with the first region.

17. The anellovector of any of the preceding embodiments, wherein the genetic element does not comprise a 5′ end or a 3′ end.

18. The anellovector of any of embodiments 15-17, wherein the genetic element does not comprise one or both of a free phosphate and a free sugar.

19. The anellovector of any of embodiments 15-18, wherein every phosphate in the genetic element is covalently linked to a first sugar by a first oxygen atom comprised by the phosphate and a second sugar by a second oxygen atom comprised by the phosphate.

20. The anellovector of any of embodiments 15-19, wherein every sugar in the genetic element is covalently linked to a first phosphate by a first carbon atom comprised by the sugar and a second phosphate by a second carbon atom comprised by the sugar.

21. The anellovector of any of embodiments 15-20, wherein the genetic element was produced by circularizing a linear RNA.

22. The anellovector of any of the preceding embodiments, wherein the genetic element is about 10-20, 20-30, 30-40, 50-60 60-70, 70-80, 80-90, 90-100, 100-125, 125-150, 150-200, 200-300, 300-400, 400-500, 500-600, 600-700, 700-800, 800-900, 900-1000, 1000-1500, 1500-2000, 2000-2500, 2500-3000, 3000-3500, 3500-4000, or 4000-4500 nucleotides in length.

23. The anellovector of any of the preceding embodiments, wherein the genetic element is at least about 10, 20, 30, 40, 50, 60, 70, 80, 90, 100, 125, 150, 175, 200, 250, 300, 400, 500, 600, 700, 800, 900, 1000, 1500, 2000, 2500, 3000, 3500, 4000, or 4500 nucleotides in length.

24. The anellovector of any of the preceding embodiments, wherein the genetic element binds the ORF1 molecule.

25. The anellovector of any of the preceding embodiments, wherein the genetic element binds a jelly roll domain comprised by the ORF1 molecule.

26. The anellovector of any of the preceding embodiments, wherein the genetic element binds an arginine-rich domain comprised by the ORF1 molecule.

27. The anellovector of any of the preceding embodiments, wherein the anellovector comprises a plurality of genetic elements, e.g., at least 2, 3, 4, 5, 10, 20, 30, 40, 50, or 60 genetic elements.

28. The anellovector of embodiment 27, wherein the genetic elements of the plurality each comprise the same sequence.

29. The anellovector of embodiment 27, wherein the genetic elements of the plurality comprise different sequences.

30. The anellovector of any of the preceding embodiments, wherein the genetic element encodes an exogenous effector.

31. The anellovector of embodiment 30, wherein the sequence encoding the exogenous effector is at least about 10, 20, 30, 40, 50, 60, 70, 80, 90, 100, 125, 150, 175, 200, 250, 300, 400, 500, 600, 700, 800, 900, 1000, 1500, 2000, 2500, or 3000 nucleotides in length.

32. The anellovector of any of embodiments 30-31, wherein the exogenous effector comprises a therapeutic effector (e.g., a polypeptide or a nucleic acid molecule).

33. The anellovector of any of embodiments 30-32, wherein the exogenous effector comprises a human protein, or a polypeptide comprising an amino acid sequence having at least 75%, 80%, 85%, 90%, 95%, 96%, 97%, 98%, or 99% sequence identity thereto.

34. The anellovector of any of embodiments 30-33, wherein the exogenous effector comprises a nucleic acid molecule.

35. The anellovector of any of embodiments 30-34, wherein the exogenous effector comprises a noncoding nucleic acid molecule, e.g., a functional RNA, e.g., an mRNA, miRNA, or siRNA).

36. The anellovector of any of embodiments 30-35, wherein the genetic element is an mRNA molecule encoding the exogenous effector (e.g., a peptide or polypeptide, e.g., a therapeutic peptide or polypeptide).

37. The anellovector of embodiment 36, wherein the noncoding nucleic acid molecule is a human noncoding nucleic acid molecule, or a nucleic acid molecule comprising a sequence having at least 75%, 80%, 85%, 90%, 95%, 96%, 97%, 98%, or 99% sequence identity thereto.

38. The anellovector of any of embodiments 30-37, wherein the exogenous effector comprises a cytosolic polypeptide or cytosolic peptide (e.g., a DPP-4 inhibitor, an activator of GLP-1 signaling, or an inhibitor of neutrophil elastase, or a functional fragment thereof).

39. The anellovector of embodiment 38, wherein the exogenous effector comprises a regulatory intracellular polypeptide.

40. The anellovector of any of embodiments 30-39, wherein the exogenous effector comprises a secreted polypeptide or peptide (e.g., a cytokine, an antibody molecule, a hormone, a growth factor, or a clotting-associated factor, or a functional fragment thereof).

41. The anellovector of any of embodiments 30-40, wherein the exogenous effector comprises a protein replacement therapeutic.

42. The anellovector of any of embodiments 30-41, wherein the exogenous effector comprises an enzyme.

43. The anellovector of any of embodiments 30-42, wherein the exogenous effector comprises erythropoietin (EPO) or human growth hormone (hGH), or a functional fragment thereof 44. The anellovector of any of embodiments 30-43, wherein the exogenous effector comprises a component of a gene editing system (e.g., a component of a CRISPR system, e.g., a Cas9, Cpf1, or a functional fragment thereof).

45. The anellovector of any of the preceding embodiments, wherein the RNA comprises chemically modified RNA, e.g., as described herein.

46. The anellovector of any of the preceding embodiments, wherein the RNA comprises a cap.

47. The anellovector of any of the preceding embodiments, wherein the RNA comprises a poly-A tail, e.g., at least about 5, 10, 15, 20, 25, 30, 40, 50, 60, 70, 80, 90, or 100 adenosines in length.

48. The anellovector of any of the preceding embodiments, wherein the RNA lacks a poly-A tail, e.g., comprises no more than 5, 10, 15, 20, 25, 30, 40, 50, 60, 70, 80, 90, or 100 sequential adenosines.

49. The anellovector of any of the preceding embodiments, wherein the proteinaceous exterior comprises about 60 (e.g., about 40, 50, 60, 70, or 80) copies of the ORF1 molecule.

50. The anellovector of any of the preceding embodiments, wherein the jelly roll domains of the ORF1 molecules face the interior of the proteinaceous exterior.

51. The anellovector of any of the preceding embodiments, wherein the ORF1 molecule comprises an amino acid sequence as listed in any of Tables N-S and 37A-37C, or an amino acid sequence having at least 30%, 40%, 50%, 60%, 70%, 75%, 80%, 85%, 90%, 95%, 96%, 97%, 98%, or 99% sequence identity thereto.

52. The anellovector of any of the preceding embodiments, wherein the ORF1 molecule comprises an arginine-rich region, e.g., comprising the amino acid sequence of an arginine-rich region as listed in any of Tables N-S and 37A-37C, or an amino acid sequence having at least 30%, 40%, 50%, 60%, 70%, 75%, 80%, 85%, 90%, 95%, 96%, 97%, 98%, or 99% sequence identity thereto.

53. The anellovector of embodiment 52, wherein the arginine-rich region comprises at least about 70% (e.g., at least about 70%, 75%, 80%, 85%, 90%, 95%, or 100%) basic residues (e.g., arginine or lysine).

54. The anellovector of any of the preceding embodiments, wherein the ORF1 molecule comprises a jelly roll domain, e.g., comprising the amino acid sequence of a jelly roll domain as listed in any of Tables N-S and 37A-37C, or an amino acid sequence having at least 30%, 40%, 50%, 60%, 70%, 75%, 80%, 85%, 90%, 95%, 96%, 97%, 98%, or 99% sequence identity thereto.

55. The anellovector of embodiment 54, wherein the jelly roll domain comprises one or more (e.g., 1, 2, 3, or 4) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

56. The anellovector of embodiment 54, wherein the jelly roll domain comprises two (3-sheets, e.g., arranged in antiparallel orientation relative to each other.

57. The anellovector of embodiment 54, wherein the jelly roll domain comprises eight β-strands.

58. The anellovector of any of embodiments 52-57, wherein the jelly roll domain comprises a region having at least 75%, 80%, 85%, 90%, 95%, 96%, 97%, 98%, 99%, or 100% sequence identity to the sequence of a D β-strand, e.g., as shown in FIG. 3.

59. The anellovector of embodiment 52, wherein the D β-strand comprises 1, 2, or 3, or more basic residues (e.g., arginine or lysine).

60. The anellovector of any of embodiments 52-59, wherein the jelly roll domain comprises a region having at least 75%, 80%, 85%, 90%, 95%, 96%, 97%, 98%, 99%, or 100% sequence identity to the sequence of a G β-strand, e.g., as shown in FIG. 3.

61. The anellovector of embodiment 52, wherein the G β-strand comprises at least about 1, 2, or 3, or more basic residues (e.g., arginine or lysine).

62. The anellovector of any of the preceding embodiments, wherein the ORF1 molecule comprises an N22 domain, e.g., comprising the amino acid sequence of an N22 domain as listed in any of Tables N-S and 37A-37C, or an amino acid sequence having at least 30%, 40%, 50%, 60%, 70%, 75%, 80%, 85%, 90%, 95%, 96%, 97%, 98%, or 99% sequence identity thereto.

63. The anellovector of any of the preceding embodiments, wherein the N22 domain comprises the amino acid sequence YNPX²DXGX²N, wherein Xⁿ is each independently a contiguous sequence of any n amino acids.

64. The anellovector of any of the preceding embodiments, wherein the N22 domain comprises a first beta strand and a second beta strand flanking the amino acid sequence YNPX²DXGX²N, e.g., wherein the first beta strand comprises the tyrosine (Y) residue of the amino acid sequence YNPX²DXGX²N and/or wherein the second beta strand comprises the second asparagine (N) residue (from N to C) of the amino acid sequence YNPX²DXGX²N.

65. The anellovector of any of the preceding embodiments, wherein the ORF1 molecule comprises a C-terminal domain, e.g., comprising the amino acid sequence of a C-terminal domain as listed in any of Tables N-S and 37A-37C, or an amino acid sequence having at least 30%, 40%, 50%, 60%, 70%, 75%, 80%, 85%, 90%, 95%, 96%, 97%, 98%, or 99% sequence identity thereto.

66. The anellovector of any of the preceding embodiments, wherein the genetic element lacks a sequence encoding an Anellovirus ORF1 protein (e.g., as described herein).

67. The anellovector of any of the preceding embodiments, wherein the genetic element lacks a sequence encoding an Anellovirus ORF2 protein (e.g., as described herein).

68. The anellovector of any of the preceding embodiments, wherein the genetic element lacks a sequence encoding an Anellovirus ORF3 protein (e.g., as described herein).

69. The anellovector of any of the preceding embodiments, wherein the anellovector is configured to deliver the genetic element to a cell (e.g., a eukaryotic cell, e.g., a mammalian cell, e.g., a human cell).

70. The anellovector of embodiment 69, wherein a population of at least 1000 of the anellovectors is capable of delivering at least about 100 copies (e.g., at least 1, 2, 3, 4, 5, 10, 20, 30, 40, 50, 100, 200, 300, 400, 500, 600, 700, 800, 900, 1000, 5000, 10,000, 50,000, 100,000, 500,000, or 1,000,000 copies) of the genetic element into one or more of the cells.

71. The anellovector of embodiment 69 or 70, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering the genetic element into at least 10%, 20%, 30%, 40%, 50%, 60%, 70%, 80%, 90%, 95%, 99%, or more of a population of the cells.

72. The anellovector of any of embodiments 69-71, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering at least 1, 2, 3, 4, 5, 6, 7, 8, 9, 10, 20, 50, 100, 200, 500, 1000, 2000, 5000, 8,000, 1×10⁴, 1×10⁵, 1×10⁶, 1×10⁷ or greater copies of the genetic element per cell to a population of the cells.

73. The anellovector of any of embodiments 69-72, wherein a population of the anellovectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering 1-3, 1-4, 1-5, 1-6, 1-7, 1-8, 1-9, 1-10, 5-10, 10-20, 20-50, 50-100, 100-1000, 1000-10⁴, 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of the genetic element per cell to a population of the cells.

74. The anellovector of any of the preceding embodiments, wherein the anellovector selectively delivers the effector to, or is present at higher levels in (e.g., preferentially accumulates in), a desired cell type, tissue, or organ (e.g., bone marrow, blood, heart, GI, skin, photoreceptors in the retina, epithelial linings, or pancreas).

75. The anellovector of any of the preceding embodiments, wherein the genetic element is protected from or resistant to digestion by an RNase (e.g., by the proteinaceous exterior).

76. The anellovector of any of the preceding embodiments, wherein the genetic element enclosed within the proteinaceous exterior is resistant to endonuclease digestion, e.g., to RNase digestion.

77. The anellovector of any of the preceding embodiments, wherein the genetic element comprises a promoter element.

78. The anellovector of any of the preceding embodiments, wherein the genetic element comprises a protein binding sequence.

79. The anellovector of embodiment 78, wherein the protein binding sequence is capable of binding to the ORF1 molecule.

80. A composition comprising a plurality of the anellovectors of any of the preceding embodiments.

81. The composition of embodiment 80, wherein at least 50%, 60%, 70%, 75%, 80%, 85%, 90%, 95%, 96%, 97%, 98%, 99%, or 100% of proteinaceous exteriors comprising an ORF1 molecule in the composition comprise at least one copy of the genetic element.

82. The composition of embodiment 80, wherein at least 50%, 60%, 70%, 75%, 80%, 85%, 90%, 95%, 96%, 97%, 98%, 99%, or 100% of proteinaceous exteriors comprising an ORF1 molecule in the composition comprise at least one copy of an anellovector genetic element.

83. The composition of any of embodiments 80-82, wherein the composition comprises at least 10², 10³, 10⁴, 10⁴, 10⁵, 10⁶, or 10⁷ of the same anellovector.

84. The composition of any of embodiments 80-83, wherein the plurality comprises at least 10³, 10⁴, 10⁵, 10⁶, 10⁷, 10⁸, 10⁹, 10¹⁰, 10¹¹, 10¹², 10¹³, 10¹⁴, or 10¹⁵ anellovectors (e.g., copies of the anellovector); or wherein the composition comprises at least 10⁵, 10⁶, 10⁷, 10⁸, 10⁹, 10¹⁰, 10¹¹, 10¹², 10¹³, 10¹⁴, or 10¹⁵ anellovector genomes per mL.

85. The composition of any of embodiments 80-84, having one or more (e.g., 1, 2, 3, 4, 5, or 6) of the following characteristics:
a) the composition meets a pharmaceutical or good manufacturing practices (GMP) standard;
b) the composition was made according to good manufacturing practices (GMP);
c) the composition has a pathogen level below a predetermined reference value, e.g., is substantially free of pathogens;
d) the composition has a contaminant level below a predetermined reference value, e.g., is substantially free of contaminants (e.g., a denaturant, e.g., urea);
e) the composition has a predetermined level of non-infectious particles or a predetermined ratio of particles:infectious units (e.g., <300:1, <200:1, <100:1, or <50:1), or
f) the pharmaceutical composition has low immunogenicity or is substantially non-immunogenic, e.g., as described herein;
optionally wherein the composition comprises urea at a concentration of less than 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.

86. The composition of any of embodiments 80-85, wherein the pharmaceutical composition has a contaminant level below a predetermined reference value, e.g., is substantially free of contaminants.

87. The composition of any of embodiments 80-86, wherein the composition comprises urea at a concentration of less than 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.

88. The composition of embodiment 86 or 87, wherein the contaminant comprises one or more of the following: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and/or aggregates.

89. The composition of any of embodiments 80-88, wherein the composition comprises less than 10% (e.g., less than about 10%, 5%, 4%, 3%, 2%, 1%, 0.5%, or 0.1%) contaminant by weight.

90. The composition of any of embodiments 80-89, wherein at least 90% of proteinaceous exteriors comprise the same genetic element (e.g., the genetic element of the anellovector).

91. The composition of any of embodiments 80-90, wherein at least 90% of proteinaceous exteriors comprise the same ORF1 molecule.

92. A pharmaceutical composition comprising the anellovector or composition of any of the preceding embodiments, and a pharmaceutically acceptable carrier or excipient.

93. A method of making an anellovector, the method comprising:

(a) providing a mixture comprising:
(i) a genetic element comprising RNA, and
(ii) an ORF1 molecule; and

(b) incubating the mixture under conditions suitable for enclosing the genetic element within a proteinaceous exterior comprising the ORF1 molecule, thereby making an anellovector;

optionally wherein the mixture is not comprised in a cell.

94. The method of embodiment 93, further comprising, prior to the providing of (a), expressing the ORF1 molecule, e.g., in a host cell (e.g., an insect cell or a mammalian cell).

95. The method of embodiment 94, wherein the expressing comprising incubating a host cell (e.g., an insect cell or a mammalian cell) comprising a nucleic acid molecule (e.g., a baculovirus expression vector) encoding the ORF1 molecule under conditions suitable for producing the ORF1 molecule.

96. The method of embodiment 94 or 95, further comprising, prior to the providing of (a), purifying the ORF1 molecule expressed by the host cell.

97. A method of purifying an anellovector, the method comprising:

(a) providing an anellovector (e.g., as described herein) comprising:
(i) a genetic element, e.g., a genetic element comprising RNA, and
(ii) a proteinaceous exterior comprising an ORF1 molecule, the proteinaceous exterior enclosing the genetic element; and

(b) purifying the anellovector.

98. The method of embodiment 96 or 97, wherein the purifying (e.g., the purifying of the ORF1 molecule or the purifying of the anelloevctor) comprises affinity purification, e.g., heparin affinity purification. 99. The method of any of embodiments 96-98, wherein the purifying (e.g., the purifying of the ORF1 molecule or the purifying of the anelloevctor) comprises size exclusion chromatography (e.g., using a Tris buffer mobile phase).

100. The method of any of embodiments 96-99, wherein the purifying (e.g., the purifying of the ORF1 molecule or the purifying of the anelloevctor) comprises affinity purification (e.g., heparin affinity purification) followed by size exclusion chromatography.

101. The method of any of embodiments 96-100, wherein the purifying (e.g., the purifying of the ORF1 molecule or the purifying of the anelloevctor) comprises anion exchange chromatography (e.g., Mustang Q membrane chromatography).

102. The method of any of embodiments 96-101, wherein the purifying (e.g., the purifying of the ORF1 molecule or the purifying of the anelloevctor) comprises mixed mode chromatography (e.g., using a mixed mode resin, e.g., a Cato700 resin).

103. The method of any of embodiments 96-102, wherein the purifying (e.g., the purifying of the ORF1 molecule or the purifying of the anelloevctor) produces a composition comprising one or more virus-like particles (VLPs) comprising at least about 20, 30, 40, 50, or 60 copies, or 20-30, 30-40, 40-50, or 50-60 copies, of the ORF1 molecule.

104. The method of embodiment 103, wherein at least 75%, 80%, 90%, 95%, 96%, 97%, 98%, 99%, or 100% of the virus-like particles comprise proteinaceous exteriors that are 60mers or are particles of at least 30, 31, 32, 33, 34, or 35 nm in diameter.

105. The method of embodiment 103, wherein the composition comprises at least 10⁵, 10⁶, 10⁷, 10⁸, 10⁹, 10¹⁰ particles/mL, or comprises 10⁵-10⁶, 10⁶-10⁷, 10⁷-10⁹, 10⁸-10⁹, 10⁹-10¹⁰, or 10¹⁰-10¹¹ particles/mL (e.g., as measured by electron microscopy).

106. The method of any of embodiments 93-105, further comprising, prior to the providing of (a), incubating the ORF1 molecule under conditions suitable for disassembly of a proteinaceous exterior (e.g., a virus-like particle (VLP)) comprising the ORF1 molecule.

107. The method of embodiment 106, wherein the conditions suitable for disassembly of the proteinaceous exterior comprising the ORF1 molecule comprise incubation in the presence of a denaturant.

108. The method of any of embodiments 106-107, wherein the denaturant comprises a chaotropic agent (e.g., urea), or a detergent (e.g., SDS (e.g., 0.1% SDS), Tween, or Triton).

109. The method of any of embodiments 106-108, wherein the conditions suitable for disassembly of the proteinaceous exterior comprising the ORF1 molecule comprise a predetermined conductivity, a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), heat (e.g., temperature above about 85, 86, 87, 88, 89, 90, 91, 92, 93, 94, or 95° C.), or pH (e.g., acidic pH or basic pH).

110. The method of any of embodiments 106-109, wherein the conditions suitable for disassembly of the proteinaceous exterior comprising the ORF1 molecule comprise incubation in a solution comprising urea at a concentration of at least 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.

111. The method of any of embodiments 106-110, wherein the incubating of (b) results in at least 50%, 60%, 70%, 75%, 80%, 85%, 90%, 96%, or 100% of a population of particles comprising the ORF1 molecule or copies thereof being disassembled.

112. The method of any of embodiments 106-111, wherein the conditions suitable for disassembly of the proteinaceous exterior are sufficient to disassemble a complex (e.g., a proteinaceous exterior) comprising at least about 20, 30, 40, 50, or 60 copies, or 20-30, 30-40, 40-50, or 50-60 copies, of the ORF1 molecule.

113. The method of any of embodiments 106-112, wherein the conditions suitable for disassembly of the proteinaceous exterior result in fewer than 10⁸ remaining intact particles/mL.

114. The method of any of embodiments 106-113, further comprising, prior to the providing of (a), removing the ORF1 molecule from the conditions suitable for disassembly of the proteinaceous exterior (e.g., subjecting the ORF1 molecule to non-denaturing conditions).

115. The method of embodiment 114, wherein the removing of the ORF1 molecule from the conditions suitable for disassembly of the proteinaceous exterior comprises reducing the concentration of the denaturant, e.g., reducing the concentration of the denaturan (e.g., urea) to below 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.

116. The method of embodiment 114 or 115, wherein the removing results in the formation of one or more anellovectors each enclosing at least one copy (e.g., at least 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 copies) of the genetic element (e.g., the mRNA).

117. The method of embodiment 116, wherein the number of anellovectors enclosing the at least one copy of the genetic element in the resulting solution is at least 10⁵, 10⁶, 10⁷, 10⁸, 10⁹, 10¹⁰, or 10¹¹; or is between 10⁵-10⁶, 10⁶-10⁷, 10⁷-10⁹, 10⁸-10⁹, 10⁹-10¹⁰, or 10¹⁰-10¹¹ (e.g., as measured by electron microscopy).

118. The method of embodiment 116, wherein the number of anellovectors enclosing the at least one copy of the genetic element in the resulting solution is at least 10⁵, 10⁶, 10⁷, 10⁸, 10⁹, 10¹⁰, or 10¹¹ anellovectors/mL; or is between 10⁵-10⁶, 10⁶-10⁷, 10⁷-10⁹, 10⁸-10⁹, 10⁹-10¹⁰, or 10¹⁰-10¹¹ anellovectors/mL (e.g., as measured by electron microscopy).

119. The method of any of embodiments 114-118, wherein the removing results in a solution comprising at least 10⁵, 10⁶, 10⁷, 10⁸, 10⁹, 10¹⁰, or 10¹¹ anellovectors/mL; or between 10⁵-10⁶, 10⁶-10⁷, 10⁷-10⁹, 10⁸-10⁹, 10⁹-10¹⁰, or 10¹⁰-10¹¹ anellovectors/mL (e.g., as measured by electron microscopy), wherein the anellovectors each enclose at least one copy (e.g., at least 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 copies) of the genetic element (e.g., the mRNA)

120. The method of any of embodiments 114-119, wherein at least 75%, 80%, 90%, 95%, 96%, 97%, 98%, 99%, or 100% of the anellovectors comprise proteinaceous exteriors that are 60mers or particles or are particles of at least 30, 31, 32, 33, 34, or 35 nm in diameter.

121. The method of any of embodiments 93-120, wherein the genetic element of the anellovector is resistant to an endonuclease (e.g., an RNase).

122. The method of any of embodiments 93-121, wherein (a) comprises admixing (i) and (ii).

123. The method of any of embodiments 93-122, which is performed in a cell-free system.

124. A method of manufacturing an anellovector composition, comprising:
(a) providing a plurality of anellovectors or compositions according to any of the preceding embodiments;
(b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and
(c) formulating the plurality of anellovectors, e.g., as a pharmaceutical composition suitable for administration to a subject, e.g., if one or more of the parameters of (b) meet a specified threshold.

125. A method of treating a disease or disorder in a subject in need thereof, the method comprising administering to the subject an anellovector or composition of any of the preceding embodiments, thereby treating a disease or disorder (e.g., as described herein) in the subject.

126. A method of modulating, e.g., enhancing or inhibiting, a biological function (e.g., as described herein) in a subject, the method comprising administering the anellovector or the composition of any of the preceding embodiments to the subject.

127. A method of delivering a genetic element to a cell, the method comprising contacting the anellovector or composition of any of the preceding embodiments with a cell, e.g., a eukaryotic cell, e.g., a mammalian cell, e.g., a human cell.

128. Use of the anellovector or composition of any of the preceding embodiments for treating a disease or disorder (e.g., as described herein) in a subject.

129. The anellovector or composition of any of the preceding embodiments for use in a method for treating a disease or disorder (e.g., as described herein) in a subject.

130. The anellovector or composition of any of the preceding embodiments for use in the manufacture of a medicament for treating a disease or disorder (e.g., as described herein) in a subject.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

FIG. 1 is a series of electron micrographs showing that recombinant capsid proteins from anellovirus strains form virus-like particles (VLPs) in vitro. Capsid proteins produced in different cell lines form VLPs in vitro as observed by negative staining electron microscopy. (A) Ring 2 ORF1 VLP purified from insect cells with an observed diameter of approximately 35 nm. (B) Ring 10 ORF1 VLP purified from insect cells with an observed diameter of approximately 35 nm. (C) CAV VP1 VLP purified from mammalian cells with an observed diameter approximately 20 nm.

FIG. 2 is a diagram showing the eight beta-strand jelly roll domain (or jelly roll fold) observed in the structure of Beak and Feather Disease Virus (BFDV). By convention, the beta strands are labeled B though I. The strands form four antiparallel beta sheets with an orientation of B-I-D-G and C-H-E-F. The B-I-D-G sheet forms the interior of the viral capsid.

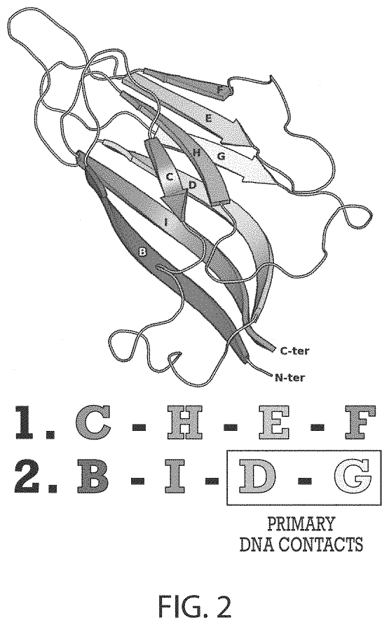

FIG. 3 is an amino acid sequence alignment depicting the jelly roll sequences for Anellovirus ORF1 protein as compared to jelly roll domain of Beak and Feather Disease Virus (BFDV)/Hepatitis E capsid protein (HepE).

FIGS. 4A-4E are a series of diagrams showing an exemplary method of producing Anellovirus ORF1 protein-based virus-like particles (VLPs) enclosing mRNA molecules encoding eGFP. (A) ORF1 protein was produced in cells and isolated as described herein. (B) VLPs that formed from the ORF1 proteins were then disassembled in a 2 M urea solution. (C) When the urea was removed in the absence of mRNA, few VLPs reformed (titer of less than 10⁸ particles/mL detected by electron microscopy). (D, E) When the urea was removed in the presence of mRNAs encoding eGFP, substantial quantities of VLPs (titer of 1×10⁹-1×10¹⁰ particles/mL) were detected by electron microscopy (EM).

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

### DETAILED DESCRIPTION OF THE INVENTION

### Definitions

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

The term “amplification,” as used herein, refers to replication of a nucleic acid molecule or a portion thereof, to produce one or more additional copies of the nucleic acid molecule or a portion thereof (e.g., a genetic element or a genetic element region). In some embodiments, amplification results in partial replication of a nucleic acid sequence. In some embodiments, amplification occurs via rolling circle replication.

As used herein, the term “anellovector” refers to a vehicle comprising a genetic element, e.g., an RNA, e.g., a circular RNA, enclosed in a proteinaceous exterior. In some embodiments, the genetic element is substantially protected from digestion with RNase by a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with an RNase, e.g., prior to entry into a host cell. In some embodiments, the anellovector is purified, e.g., it is separated from its original source and/or substantially free (>50%, >60%, >70%, >80%, >90%) of other components. In some embodiments, the anellovector is capable of introducing the genetic element into a target cell (e.g., via infection). In some embodiments, the anellovector is an infective synthetic viral particle containing certain Anellovirus elements, such as an Anellovirus ORF1 molecule.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

The term “backbone” or “backbone region,” as used herein, refers to a region within a nucleic acid molecule (e.g., within a bacmid or donor vector, e.g., as described herein) that comprises one or more elements involved in (e.g., necessary and/or sufficient for) replication and/or maintenance of the nucleic acid molecule in a host cell. In some embodiments, a backbone region, such as a “baculovirus backbone region,” comprises one or more baculoviral elements (e.g., a baculovirus genome or a functional fragment thereof), e.g., suitable for replication of the nucleic acid construct in insect cells (e.g., Sf9 cells). In some embodiments, the backbone further comprises a selectable marker. In some embodiments, a nucleic acid molecule comprises a genetic element region and a backbone region (e.g., a baculovirus backbone region and/or a backbone region suitable for replication in bacterial cells).

The term “bacmid”, as used herein, refers to a nucleic acid molecule comprising sufficient baculovirus backbone elements such that it is suitable for replication in insect cells, and furthermore is suitable for replication in bacterial cells. In some embodiments, the nucleic acid molecule is suitable for replication in bacterial cells (e.g., E. coli cells, e.g., DH 10Bac cells).

As used herein, a “circular” nucleic acid refers to a nucleic acid that forms a structure without free 5′ or 3′ ends. In some embodiments, the circular nucleic acid is closed through covalent or non-covalent bonds. For instance, the circular nucleic acid may be made by covalently linking the ends of a linear nucleic acid, e.g., with a phosphate-sugar bond or a synthetic linker moeity. In other embodiments, the circular nucleic acid comprises two ends that are in proximity and are not free (not substantially accessible to an exonuclease). For instance, the circular nucleic acid may be made by hybridizing the ends of a linear nucleic acid directly or through a nucleic acid splint.

As used herein, a “DNA region” refers to a portion of a polynucleotide strand comprising a plurality of DNA nucleotides. For example, in some embodiments a DNA region is a plurality of DNA nucleotides incorporated into an RNA strand. For example, a DNA region comprises about 5-10, 10-20, 20-30, 30-40, 40-50, 50-60, 60-70, 70-80, 80-90, or 90-100 DNA nucleotides within a polynucleotide strand.

As used herein, a nucleic acid “encoding” refers to a nucleic acid sequence encoding an amino acid sequence or a polynucleotide, e.g., an mRNA or functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid molecule that is or can be enclosed within (e.g, protected from RNase digestion by) a proteinaceous exterior, e.g., to form an anellovector as described herein. It is understood that the genetic element can be produced as naked RNA and optionally further assembled into a proteinaceous exterior. It is also understood that an anellovector can insert its genetic element into a cell, resulting in the genetic element being present in the cell and the proteinaceous exterior not necessarily entering the cell.

As used herein, “genetic element construct” refers to a nucleic acid construct (e.g., a plasmid, bacmid, donor vector, cosmid, or minicircle) comprising a genetic element sequence, or fragment thereof. In some embodiments, a bacmid or donor vector as described herein is a genetic element construct comprising a genetic element sequence, or fragment thereof.

The term “genetic element region,” as used herein, refers to a region of a construct that comprises the sequence of a genetic element. In some embodiments, the genetic element region comprises a sequence having sufficient identity to a wild-type Anellovirus sequence, or a fragment thereof, to be enclosed by a proteinaceous exterior, thereby forming an anellovector (e.g., a sequence having at least 70%, 75%, 80%, 85%, 90%, 95%, 96%, 97%, 98%, 99%, or 100% sequence identity to the wild-type Anellovirus sequence or fragment thereof). In embodiments, the genetic element region comprises a protein binding sequence, e.g., as described herein (e.g., a 5′ UTR, 3′ UTR, and/or a GC-rich region as described herein, or a sequence having at least 70%, 75%, 80%, 85%, 90%, 95%, 96%, 97%, 98%, 99%, or 100% sequence identity thereto). In some embodiments, the construct comprising a genetic element region is not enclosed in a proteinaceous exterior, but a genetic element produced from the construct can be enclosed in a proteinaceous exterior. In some embodiments, the construct comprising the genetic element region further comprises a vector backbone (e.g., a bacmid backbone or a donor vector backbone). In some embodiments, the construct (e.g., bacmid) comprises one or more baculovirus elements (e.g., a baculovirus genome, e.g., comprising the genetic element region).

As used herein, the term “mutant” when used with respect to a genome (e.g., an Anellovirus genome), or a fragment thereof, refers to a sequence having at least one change relative to a corresponding wild-type Anellovirus sequence. In some embodiments, the mutant genome or fragment thereof comprises at least one single nucleotide polymorphism, addition, deletion, or frameshift relative to the corresponding wild-type Anellovirus sequence. In some embodiments, the mutant genome or fragment thereof comprises a deletion of at least one Anellovirus ORF (e.g., one or more of ORF1, ORF2, ORF2/2, ORF2/3, ORF1/1, and/or ORF1/2) relative to the corresponding wild-type Anellovirus sequence. In some embodiments, the mutant genome or fragment thereof comprises a deletion of all Anellovirus ORFs (e.g., all of ORF1, ORF2, ORF2/2, ORF2/3, ORF1/1, and ORF1/2) relative to the corresponding wild-type Anellovirus sequence. In some embodiments, the mutant genome or fragment thereof comprises a deletion of at least one Anellovirus noncoding region (e.g., one or more of a 5′ UTR, 3′ UTR, and/or GC-rich region) relative to the corresponding wild-type Anellovirus sequence. In some embodiments, the mutant genome or fragment thereof comprises or encodes an exogenous effector.

As used herein the term “ORF molecule” refers to a polypeptide having an activity and/or a structural feature of an Anellovirus ORF protein (e.g., a polypeptide comprising an Anellovirus ORF1, ORF2, ORF2/2, ORF2/3, ORF1/1, and/or ORF1/2 protein), or a functional fragment thereof. When used generically (i.e., “ORF molecule”), the polypeptide may comprise an activity and/or structural feature of any of the Anellovirus ORFs described herein (e.g., any of an Anellovirus ORF1, ORF2, ORF2/2, ORF2/3, ORF1/1, and/or ORF1/2), or a functional fragment thereof. When used with a modifier to indicate a particular open reading frame (e.g., “ORF1 molecule,” “ORF2 molecule,” “ORF2/2 molecule,” “ORF2/3 molecule,” “ORF1/1 molecule,” or “ORF1/2 molecule”), it is generally meant that the polypeptide comprises an activity and/or structural feature of the corresponding Anellovirus ORF protein, or a functional fragment thereof (for example, as defined below for “ORF1 molecule”). For example, an “ORF2 molecule” comprises an activity and/or structural feature of an Anellovirus ORF2 protein, or a functional fragment thereof.

As used herein, the term “ORF1 molecule” refers to a polypeptide having an activity and/or a structural feature of an Anellovirus ORF1 protein (e.g., an Anellovirus ORF1 protein as described herein), or a functional fragment thereof. An ORF1 molecule may, in some instances, comprise one or more of (e.g., 1, 2, 3 or 4 of): a first region comprising at least 60% basic residues (e.g., at least 60% arginine residues), a second region comprising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid. An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein).

As used herein, the term “ORF2 molecule” refers to a polypeptide having an activity and/or a structural feature of an Anellovirus ORF2 protein (e.g., an Anellovirus ORF2 protein as described herein), or a functional fragment thereof. An “Anellovirus ORF2 protein,” as used herein, refers to an ORF2 protein encoded by an Anellovirus genome (e.g., a wild-type Anellovirus genome, e.g., as described herein).

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In some embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, a “substantially non-pathogenic” organism, particle, or component, refers to an organism, particle (e.g., a virus or an anellovector, e.g., as described herein), or component thereof that does not cause or induce an unacceptable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce an undesirable condition (e.g., a disease or pathogenic condition), e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization. In some embodiments, integration frequency is determined by quantitative gel purification assay of genomic DNA separated from free vector, e.g., as described in Wang et al. (2004, Gene Therapy 11: 711-721, incorporated herein by reference in its entirety).

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a clinically significant immune response. In some embodiments, the substantially non-immunogenic anellovector does not produce a clinically significant immune response against a protein comprising an amino acid sequence or encoded by a nucleic acid sequence of an Anellovirus or anellovector genetic element. In some embodiments, an immune response (e.g., an undesired or untargeted immune response) is detected by assaying antibody (e.g., neutralizing antibody) presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies (e.g., neutralizing antibodies) against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

This invention relates generally to anellovectors, e.g., synthetic anellovectors, and uses thereof. The present disclosure provides anellovectors, compositions comprising anellovectors, and methods of making or using anellovectors. Anellovectors are generally useful as delivery vehicles, e.g., for delivering a therapeutic agent to a eukaryotic cell. Generally, an anellovector described herein will include a genetic element comprising an RNA sequence (e.g., an RNA sequence encoding an effector, e.g., an exogenous effector or an endogenous effector) enclosed within a proteinaceous exterior. An anellovector may include one or more deletions of sequences (e.g., regions or domains as described herein) relative to an Anellovirus sequence (e.g., as described herein). Anellovectors can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

TABLE OF CONTENTS

```
I. Compositions and Methods for Making Anellovectors by In Vitro
Assembly
  A. Components and Assembly of Anellovectors
    i. ORF1 molecules for assembly of Anellovectors
    ii. ORF2 molecules for assembly of Anellovectors
  B. Genetic Elements
    i. Genetic Elements comprising RNA
      a. RNA-only genetic elements
      b. Hybridized RNA-ssDNA genetic elements
      c. RNA/DNA conjugates
  C. Genetic Element Constructs
  D. Production of RNA-based genetic elements
  E. Production of protein components
    i. Baculovirus expression systems
    ii. Insect cell systems
    iii. Mammalian cell systems
  F. Effectors
  G. In vitro assembly methods
  H. Enrichment and Purification
II. Anellovectors
  A. Anelloviruses
  B. ORF1 molecules
  C. ORF2 molecules
  D. Genetic elements
  E. Protein binding sequences
  F. 5′ UTR regions
  G. GC-rich regions
  H. Effectors
  I. Regulatory Sequences
  J. Other Sequences
  K. Proteinaceous exterior
III. Methods of use
IV. Administration/Delivery
```

### I. Compositions and Methods for Making Anellovectors by In Vitro Assembly

The present disclosure provides, in some aspects, compositions and methods that can be used for producing anellovectors, e.g., anellovectors having a genetic element comprising RNA, as described herein. In some embodiments, the compositions and methods described herein can be used to produce a genetic element or a genetic element construct. In some embodiments, the compositions and methods described herein can be used to produce a genetic element or a genetic element construct by in vitro assembly. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell (e.g., an insect cell, e.g., an Sf9 cell).

### Components and Assembly of Anellovectors

The compositions and methods herein can be used to produce anellovectors. As described herein, an anellovector generally comprises a genetic element (e.g., an RNA molecule) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, the genetic element is enclosed within the proteinaceous exterior in a host cell (e.g., an insect cell, e.g., an Sf9 cell). In some embodiments, the host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, the host cell comprises the genetic element and/or a nucleic acid construct comprising the sequence of the genetic element. In some embodiments, the nucleic acid construct is selected from a plasmid, viral nucleic acid, minicircle, bacmid, or artificial chromosome. In some embodiments, the genetic element is excised from the nucleic acid construct (e.g., bacmid) and, optionally, converted from a double-stranded form to a single-stranded form (e.g., by denaturation). In some embodiments, the genetic element is generated by a polymerase based on a template sequence in the nucleic acid construct (e.g., bacmid). In some embodiments, the polymerase produces a single-stranded copy of the genetic element sequence, which can optionally be circularized to form a genetic element as described herein.

In some embodiments, the host cell comprises a genetic element construct (e.g., a bacmid, plasmid, or minicircle) and a bacmid comprising one or more sequences encoding Anellovirus ORF molecules (e.g., ORF1, ORF2, ORF2/2, ORF2/3, ORF1/1, and/or ORF1/2 ORF molecules), or functional fragments thereof. In some embodiments, proteinaceous exterior proteins are expressed from the bacmid. In embodiments, the proteinaceous exterior proteins expressed from the bacmid enclose a genetic element, thereby forming an anellovector. In some embodiments, the bacmid comprises a backbone suitable for replication of the nucleic acid construct in insect cells (e.g., Sf9 cells), e.g., a baculovirus backbone region. In some embodiments, the bacmid comprises a backbone region suitable for replication of the genetic element construct in bacterial cells (e.g., E. coli cells, e.g., DH 10Bac cells). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells). In some embodiments, the bacmid is introduced into the host cell via a baculovirus particle. In embodiments, the bacmid is produced by a producer cell, e.g., an insect cell (e.g., an Sf9 cell) or a bacterial cell (e.g., an E. coli cell, e.g., a DH 10Bac cell). In embodiments, the producer cell comprises a bacmid and/or a donor vector, e.g., as described herein. In embodiments, the producer cell further comprises sufficient cellular machinery for replication of the bacmid and/or donor vector.

ORF1 Molecules, e.g., for Assembly of Anellovectors

An anellovector can be made, for example, by enclosing a genetic element within a proteinaceous exterior. In some embodiments, the enclosure occurs in a cell-free system or in a cell. The proteinaceous exterior of an Anellovector generally comprises a polypeptide encoded by an Anellovirus ORF1 nucleic acid (e.g., an Anellovirus ORF1 molecule or a splice variant or functional fragment thereof, e.g., as described herein). An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 arginine-rich region, jelly-roll region, N22 domain, hypervariable region, and/or C-terminal domain. In some embodiments, the proteinaceous exterior comprises an Anellovirus ORF1 jelly-roll region (e.g., as described herein). In some embodiments, the proteinaceous exterior comprises an Anellovirus ORF1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer.

ORF2 Molecules, e.g., for Assembly of Anellovectors

Producing an anellovector using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector does not comprise an ORF2 molecule, or a splice variant or functional fragment thereof, and/or a nucleic acid encoding an ORF2 molecule, or a splice variant or functional fragment thereof. In some embodiments, producing the anellovector comprises expression of an ORF2 molecule, or a splice variant or functional fragment thereof, but the ORF2 molecule is not incorporated into the anellovector.

### Genetic Elements

Genetic Elements Comprising RNA

In some embodiments, a genetic element is or comprises a nucleic acid. In some embodiments, a genetic element is a single-stranded polynucleotide. In some embodiments, a genetic element comprises one or more double stranded regions. In some embodiments, a genetic element comprises RNA. In some embodiments, the genetic element comprises an RNA hairpin structure. In some embodiments, the genetic element is an mRNA, e.g., a chemically modified mRNA. In some embodiments, a genetic element consists of at least 10%, 20%, 25%, 30%, 40%, 50%, 60%, 70%, 75%, 80%, 85%, 90%, 95%, 96%, 97%, 98%, 99%, or 100% RNA. In some embodiments, the genetic element comprises a DNA strand and an RNA strand, e.g., wherein at least a portion of the DNA strand hybridizes to at least a portion of the RNA strand.

In some embodiments, the genetic element does not encode any of an Anellovirus ORF1, ORF1/1, ORF1/2, ORF2, ORF2/2, ORF2/3, or ORF2t/3.

In some embodiments, the RNA genetic element encodes an effector, e.g., an effector protein.

In some embodiments, the RNA genetic element is or comprises an effector, e.g., a functional RNA. In some embodiments, RNA is selected from the group consisting of mRNA, rRNA, tRNA (e.g., a TREM), regulatory RNA, non-coding RNA, long non-coding RNA (lncRNA), circular RNA (circRNA), double stranded RNA (dsRNA), guide RNA (gRNA), small interfering RNA (siRNA), short hairpin RNA (shRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), and other RNAi molecules.

In some embodiments, the genetic element comprises RNA, e.g., chemically modified RNA. In some embodiments, one or more nucleotides of RNA of a genetic element are chemically modified. In some embodiments, RNA comprises one or more chemical modifications to one or more bases. In some embodiments, RNA comprises one or more chemical modifications to one or more sugars. In some embodiments, at least 1, 2, 3, 4, 5, 6, 7, 8, 9, or 10 nucleotides of RNA of a genetic element are chemically modified. In some embodiments, RNA comprises one or more backbone modifications. In some embodiments, a modification comprises a non-naturally occurring modification, e.g., a modification described in any one of Tables 5-9. A non-naturally occurring modification can be made according to methods known in the art.

In some embodiments, a genetic element described herein comprises a non-naturally occurring modification provided in Table 5, or a combination thereof.

TABLE 5
Exemplary non-naturally occurring modifications

| | | | Naturally |
|---|---|---|---|
| Name | Symbol | Base | Occurring |
| 7-deaza-adenosine | — | A | NO |
| Nl-methyl-adenosine | — | A | NO |
| N6,N6 (dimethyl)adenine | — | A | NO |
| N6-cis-hydroxy-isopentenyl-adenosine | — | A | NO |
| a-thio-adenosine | — | A | NO |
| 2 (amino)adenine | — | A | NO |
| 2 (aminopropyl)adenine | — | A | NO |
| 2 (methylthio) N6 (isopentenyl)adenine | — | A | NO |
| 2-(alkyl)adenine | — | A | NO |
| 2-(aminoalkyl)adenine | — | A | NO |
| 2-(aminopropyl)adenine | — | A | NO |
| 2-(halo)adenine | — | A | NO |
| 2-(halo)adenine | — | A | NO |
| 2-(propyl)adenine | — | A | NO |
| 2′-Amino-2′-deoxy-ATP | — | A | NO |
| 2′-Azido-2′-deoxy-ATP | — | A | NO |
| 2′-Deoxy-2′-a-aminoadenosine TP | — | A | NO |
| 2′-Deoxy-2′-a-azidoadenosine TP | — | A | NO |
| 6 (alkyl)adenine | — | A | NO |
| 6 (methyl)adenine | — | A | NO |
| 6-(alkyl)adenine | — | A | NO |
| 6-(methyl)adenine | — | A | NO |
| 7 (deaza)adenine | — | A | NO |
| 8 (alkenyl)adenine | — | A | NO |
| 8 (alkynyl)adenine | — | A | NO |
| 8 (amino)adenine | — | A | NO |
| 8 (thioalkyl)adenine | — | A | NO |
| 8-(alkenyl)adenine | — | A | NO |
| 8-(alkyl)adenine | — | A | NO |
| 8-(alkynyl)adenine | — | A | NO |
| 8-(amino)adenine | — | A | NO |
| 8-(halo)adenine | — | A | NO |
| 8-(hydroxyl)adenine | — | A | NO |
| 8-(thioalkyl)adenine | — | A | NO |
| 8-(thiol)adenine | — | A | NO |
| 8-azido-adenosine | — | A | NO |
| aza adenine | — | A | NO |
| deaza adenine | — | A | NO |
| N6 (methyl)adenine | — | A | NO |
| N6-(isopentyl)adenine | — | A | NO |
| 7-deaza-8-aza-adenosine | — | A | NO |
| 7-methyladenine | — | A | NO |
| 1-Deazaadenosine TP | — | A | NO |
| 2′Fluoro-N6-Bz-deoxyadenosine TP | — | A | NO |
| 2′-OMe-2-Amino-ATP | — | A | NO |
| 2′O-methyl-N6-Bz-deoxyadenosine TP | — | A | NO |
| 2′-a-Ethynyladenosine TP | — | A | NO |
| 2-aminoadenine | — | A | NO |
| 2-Aminoadenosine TP | — | A | NO |
| 2-Amino-ATP | — | A | NO |
| 2′-a-Trifluoromethyladenosine TP | — | A | NO |
| 2-Azidoadenosine TP | — | A | NO |
| 2′-b-Ethynyladenosine TP | — | A | NO |
| 2-Bromoadenosine TP | — | A | NO |
| 2′-b-Trifluoromethyladenosine TP | — | A | NO |
| 2-Chloroadenosine TP | — | A | NO |
| 2′-Deoxy-2′,2′-difluoroadenosine TP | — | A | NO |
| 2′-Deoxy-2′-a-mercaptoadenosine TP | — | A | NO |
| 2′-Deoxy-2′-a-thiomethoxyadenosine TP | — | A | NO |
| 2′-Deoxy-2′-b-aminoadenosine TP | — | A | NO |
| 2′-Deoxy-2′-b-azidoadenosine TP | — | A | NO |
| 2′-Deoxy-2′-b-bromoadenosine TP | — | A | NO |
| 2′-Deoxy-2′-b-chloroadenosine TP | — | A | NO |
| 2′-Deoxy-2′-b-fluoroadenosine TP | — | A | NO |
| 2′-Deoxy-2′-b-iodoadenosine TP | — | A | NO |
| 2′-Deoxy-2′-b-mercaptoadenosine TP | — | A | NO |
| 2′-Deoxy-2′-b-thiomethoxyadenosine TP | — | A | NO |
| 2-Fluoroadenosine TP | — | A | NO |
| 2-Iodoadenosine TP | — | A | NO |
| 2-Mercaptoadenosine TP | — | A | NO |
| 2-methoxy-adenine | — | A | NO |
| 2-methylthio-adenine | — | A | NO |
| 2-Trifluoromethyladenosine TP | — | A | NO |
| 3-Deaza-3-bromoadenosine TP | — | A | NO |
| 3-Deaza-3-chloroadenosine TP | — | A | NO |
| 3-Deaza-3-fluoroadenosine TP | — | A | NO |
| 3-Deaza-3-iodoadenosine TP | — | A | NO |
| 3-Deazaadenosine TP | — | A | NO |
| 4′-Azidoadenosine TP | — | A | NO |
| 4′-Carbocyclic adenosine TP | — | A | NO |
| 4′-Ethynyladenosine TP | — | A | NO |
| 5′-Homo-adenosine TP | — | A | NO |
| 8-Aza-ATP | — | A | NO |
| 8-bromo-adenosine TP | — | A | NO |
| 8-Trifluoromethyladenosine TP | — | A | NO |
| 9-Deazaadenosine TP | — | A | NO |
| 2-aminopurine | — | A/G | NO |
| 7-deaza-2,6-diaminopurine | — | A/G | NO |
| 7-deaza-8-aza-2,6-diaminopurine | — | A/G | NO |
| 7-deaza-8-aza-2-aminopurine | — | A/G | NO |
| 2,6-diaminopurine | — | A/G | NO |
| 7-deaza-8-aza-adenine, 7-deaza-2- | — | A/G | NO |
| aminopurine | | | |
| 4-methylcytidine | — | C | NO |
| 5-aza-cytidine | — | C | NO |
| Pseudo-iso-cytidine | — | C | NO |
| pyrrolo-cytidine | — | C | NO |
| a-thio-cytidine | — | C | NO |
| 2-(thio)cytosine | — | C | NO |
| 2′-Amino-2′-deoxy-CTP | — | C | NO |
| 2′-Azido-2′-deoxy-CTP | — | C | NO |
| 2′-Deoxy-2′-a-aminocytidine TP | — | C | NO |
| 2′-Deoxy-2′-a-azidocytidine TP | — | C | NO |
| 3 (deaza) 5 (aza)cytosine | — | C | NO |
| 3 (methyl)cytosine | — | C | NO |
| 3-(alkyl)cytosine | — | C | NO |
| 3-(deaza) 5 (aza)cytosine | — | C | NO |
| 3-(methyl)cytidine | — | C | NO |
| 4,2′-O-dimethylcytidine | — | C | NO |
| 5 (halo)cytosine | — | C | NO |
| 5 (methyl)cytosine | — | C | NO |
| 5 (propynyl)cytosine | — | C | NO |
| 5 (trifluoromethyl)cytosine | — | C | NO |
| 5-(alkyl)cytosine | — | C | NO |
| 5-(alkyny!)cytosine | — | C | NO |
| 5-(halo)cytosine | — | C | NO |
| 5-(propynyl)cytosine | — | C | NO |
| 5-(trifluoromethyl)cytosine | — | C | NO |
| 5-bromo-cytidine | — | C | NO |
| 5-iodo-cytidine | — | C | NO |
| 5-propynyl cytosine | — | C | NO |
| 6-(azo)cytosine | — | C | NO |
| 6-aza-cytidine | — | C | NO |
| aza cytosine | — | C | NO |
| deaza cytosine | — | C | NO |
| N4 (acetyl)cytosine | — | C | NO |
| l-methyl-1-deaza-pseudoisocytidine | — | C | NO |
| 1-methyl-pseudoisocytidine | — | C | NO |
| 2-methoxy-5-methyl-cytidine | — | C | NO |
| 2-methoxy-cytidine | — | C | NO |
| 2-thio-5-methyl-cytidine | — | C | NO |
| 4-methoxy-1-methyl-pseudoisocytidine | — | C | NO |
| 4-methoxy-pseudoisocytidine | — | C | NO |
| 4-thio-l-methyl-1-deaza- | — | C | NO |
| pseudoisocytidine | | | |
| 4-thio-1-methyl-pseudoisocytidine | — | C | NO |
| 4-thio-pseudoisocytidine | — | C | NO |
| 5-aza-zebularine | — | C | NO |
| 5-methyl-zebularine | — | C | NO |
| pyrrolo-pseudoisocytidine | — | C | NO |
| zebularine | — | C | NO |
| (E)-5-(2-Bromo-vinyl)cytidine TP | — | C | NO |
| 2,2′-anhydro-cytidine TP hydrochloride | — | C | NO |
| 2′Fluor-N4-Bz-cytidine TP | — | C | NO |
| 2′Fluoro-N4-Acetyl-cytidine TP | — | C | NO |
| 2′-O-Methyl-N4-Acetyl-cytidine TP | — | C | NO |
| 2′O-methyl-N4-Bz-cytidine TP | — | C | NO |
| 2′-a-Ethynylcytidine TP | — | C | NO |
| 2′-a-Trifluoromethylcytidine TP | — | C | NO |
| 2′-b-Ethynylcytidine TP | — | C | NO |
| 2′-b-Trifluoromethylcytidine TP | — | C | NO |
| 2′-Deoxy-2′,2′-difluorocytidine TP | — | C | NO |
| 2′-Deoxy-2′-a-mercaptocytidine TP | — | C | NO |
| 2′-Deoxy-2′-a-thiomethoxycytidine TP | — | C | NO |
| 2′-Deoxy-2′-b-aminocytidine TP | — | C | NO |
| 2′-Deoxy-2′-b-azidocytidine TP | — | C | NO |
| 2′-Deoxy-2′-b-bromocytidine TP | — | C | NO |
| 2′-Deoxy-2′-b-chlorocytidine TP | — | C | NO |
| 2′-Deoxy-2′-b-fluorocytidine TP | — | C | NO |
| 2′-Deoxy-2′-b-iodocytidine TP | — | C | NO |
| 2′-Deoxy-2′-b-mercaptocytidine TP | — | C | NO |
| 2′-Deoxy-2′-b-thiomethoxycytidine TP | — | C | NO |
| 2′-O-Methyl-5-(1-propynyl)cytidine TP | — | C | NO |
| 3′-Ethynylcytidine TP | — | C | NO |
| 4′-Azidocytidine TP | — | C | NO |
| 4′-Carbocyclic cytidine TP | — | C | NO |
| 4′-Ethynylcytidine TP | — | C | NO |
| 5-(1-Propynyl)ara-cytidine TP | — | C | NO |
| 5-(2-Chloro-phenyl)-2-thiocytidine TP | — | C | NO |
| 5-(4-Amino-phenyl)-2-thiocytidine TP | — | C | NO |
| 5-Aminoallyl-CTP | — | C | NO |
| 5-Cyanocytidine TP | — | C | NO |
| 5-Ethynylara-cytidine TP | — | C | NO |
| 5-Ethynylcytidine TP | — | C | NO |
| 5′-Homo-cytidine TP | — | C | NO |
| 5-Methoxycytidine TP | — | C | NO |
| 5-Trifluoromethyl-Cytidine TP | — | C | NO |
| N4-Amino-cytidine TP | — | C | NO |
| N4-Benzoyl-cytidine TP | — | C | NO |
| pseudoisocytidine | — | C | NO |
| 6-thio-guanosine | — | G | NO |
| 7-deaza-guanosine | — | G | NO |
| 8-oxo-guanosine | — | G | NO |
| N1-methyl-guanosine | — | G | NO |
| a-thio-guanosine | — | G | NO |
| 2 (propyl)guanine | — | G | NO |
| 2-(alkyl)guanine | — | G | NO |
| 2′-Amino-2′-deoxy-GTP | — | G | NO |
| 2′-Azido-2′-deoxy-GTP | — | G | NO |
| 2′-Deoxy-2′-a-aminoguanosine TP | — | G | NO |
| 2′-Deoxy-2′-a-azidoguanosine TP | — | G | NO |
| 6 (methyl)guanine | — | G | NO |
| 6-(alky1)guanine | — | G | NO |
| 6-(methyl)guanine | — | G | NO |
| 6-methyl-guanosine | — | G | NO |
| 7 (alkyl)guanine | — | G | NO |
| 7 (deaza)guanine | — | G | NO |
| 7 (methyl)guanine | — | G | NO |
| 7-(alkyl)guanine | — | G | NO |
| 7-(deaza)guanine | — | G | NO |
| 7-(methyl)guanine | — | G | NO |
| 8 (alkyl)guanine | — | G | NO |
| 8 (alkynyl)guanine | — | G | NO |
| 8 (halo)guanine | — | G | NO |
| 8 (thioalkyl)guanine | — | G | NO |
| 8-(alkenyl)guanine | — | G | NO |
| 8-(alkyl)guanine | — | G | NO |
| 8-(alkynyl)guanine | — | G | NO |
| 8-(amino)guanine | — | G | NO |
| 8-(halo)guanine | — | G | NO |
| 8-(hydroxyl)guanine | — | G | NO |
| 8-(thioalkyl)guanine | — | G | NO |
| 8-(thiol)guanine | — | G | NO |
| azaguanine | — | G | NO |
| deaza guanine | — | G | NO |
| N (methyl)guanine | — | G | NO |
| N-(methyl)guanine | — | G | NO |
| l-methyl-6-thio-guanosine | — | G | NO |
| 6-methoxy-guanosine | — | G | NO |
| 6-thio-7-deaza-8-aza-guanosine | — | G | NO |
| 6-thio-7-deaza-guanosine | — | G | NO |
| 6-thio-7-methyl-guanosine | — | G | NO |
| 7-deaza-8-aza-guanosine | — | G | NO |
| 7-methyl-8-oxo-guanosine | — | G | NO |
| N2,N2-dimethyl-6-thio-guanosine | — | G | NO |
| N2-methyl-6-thio-guanosine | — | G | NO |
| 1-Me-GTP | — | G | NO |
| 2′Fluoro-N2-isobutyl-guanosine TP | — | G | NO |
| 2′O-methyl-N2-isobutyl-guanosine TP | — | G | NO |
| 2′-a-Ethynylguanosine TP | — | G | NO |
| 2′-a-Trifluoromethylguanosine TP | — | G | NO |
| 2′-b-Ethynylguanosine TP | — | G | NO |
| 2′-b-Trifluoromethylguanosine TP | — | G | NO |
| 2′-Deoxy-2′,2′-difluoroguanosine TP | — | G | NO |
| 2′-Deoxy-2′-a-mercaptoguanosine TP | — | G | NO |
| 2′-Deoxy-2′-a-thiomethoxyguanosine TP | — | G | NO |
| 2′-Deoxy-2′-b-aminoguanosine TP | — | G | NO |
| 2′-Deoxy-2′-b-azidoguanosine TP | — | G | NO |
| 2′-Deoxy-2′-b-bromoguanosine TP | — | G | NO |
| 2′-Deoxy-2′-b-chloroguanosine TP | — | G | NO |
| 2′-Deoxy-2′-b-fluoroguanosine TP | — | G | NO |
| 2′-Deoxy-2′-b-iodoguanosine TP | — | G | NO |
| 2′-Deoxy-2′-b-mercaptoguanosine TP | — | G | NO |
| 2′-Deoxy-2′-b-thiomethoxyguanosine TP | — | G | NO |
| 4′-Azidoguanosine TP | — | G | NO |
| 4′-Carbocyclic guanosine TP | — | G | NO |
| 4′-Ethynylguanosine TP | — | G | NO |
| 5′-Homo-guanosine TP | — | G | NO |
| 8-bromo-guanosine TP | — | G | NO |
| 9-Deazaguanosine TP | — | G | NO |
| N2-isobutyl-guanosine TP | — | G | NO |
| 7-methylinosine | | A | NO |
| allyamino-thymidine | — | T | NO |
| aza thymidine | — | T | NO |
| deaza thymidine | — | T | NO |
| deoxy-thymidine | — | T | NO |
| 5-propynyl uracil | — | U | NO |
| a-thio-uridine | — | U | NO |
| 1 (aminoalkylamino-carbonylethylenyl)- | — | U | NO |
| 2 (thio)-pseudouracil | | | |
| 1 (aminoalkylaminocarbonylethylenyl)- | — | U | NO |
| 2,4-(dithio)pseudouracil | | | |
| 1 (aminoalkylaminocarbonylethylenyl)-4 | — | U | NO |
| (thio)pseudouracil | | | |
| 1 (aminoalkylaminocarbonylethylenyl)- | — | U | NO |
| pseudouracil | | | |
| 1 (aminocarbonylethylenyl)-2(thio)- | — | U | NO |
| pseudouracil | | | |
| 1 (aminocarbonylethylenyl)-2,4- | — | U | NO |
| (dithio)pseudouracil | | | |
| 1 (aminocarbonylethylenyl)-4 | — | U | NO |
| (thio)pseudouracil | | | |
| 1 (aminocarbonylethylenyl)-pseudouracil | — | U | NO |
| 1 substituted 2(thio)-pseudouracil | — | U | NO |
| 1 substituted 2,4-(dithio)pseudouracil | — | U | NO |
| 1 substituted 4 (thio)pseudouracil | — | U | NO |
| 1 substituted pseudouracil | — | U | NO |
| 1-(aminoalkylamino-carbonylethylenyl)- | — | U | NO |
| 2-(thio)-pseudouracil | | | |
| l-Methyl-3-(3-amino-3-carboxypropyl) | — | U | NO |
| pseudouridine TP | | | |
| l-Methyl-3-(3-amino-3- | — | U | NO |
| carboxyproovl)pseudo-UTP | | | |
| 1-Methyl-pseudo-UTP | — | U | NO |
| 2 (thio)pseudouracil | — | U | NO |
| 2′ deoxy uridine | — | U | NO |
| 2′ fluorouridine | — | U | NO |
| 2-(thio)uracil | — | U | NO |
| 2,4-(dithio)psuedouracil | — | U | NO |
| 2′ methyl, 2′amino, 2′azido, 2′fluro- | — | U | NO |
| guanosine | | | |
| 2′-Amino-2′-deoxy-UTP | — | U | NO |
| 2′-Azido-2′-deoxy-UTP | — | U | NO |
| 2′-Azido-deoxyuridine TP | — | U | NO |
| 2′-O-methylpseudouridine | — | U | NO |
| 2′ deoxy uridine | 2′dU | U | NO |
| 2′ fluorouridine | — | U | NO |
| 2′-Deoxy-2′-a-aminouridine TP | — | U | NO |
| 2′-Deoxy-2′-a-azidouridine TP | — | U | NO |
| 2-methylpseudouridine | m3′P | U | NO |
| 3 (3 amino-3 carboxypropyl)uracil | — | U | NO |
| 4 (thio)pseudouracil | — | U | NO |
| 4-(thio )pseudouracil | — | U | NO |
| 4-(thio)uracil | — | U | NO |
| 4-thiouracil | — | U | NO |
| 5 (1,3-diazole-1-alkyl)uracil | — | U | NO |
| 5 (2-aminopropyl)uracil | — | U | NO |
| 5 (aminoalkyl)uracil | — | U | NO |
| 5 (dimethylaminoalkyl)uracil | — | U | NO |
| 5 (guanidiniumalkyl)uracil | — | U | NO |
| 5 (methoxycarbonylmethyl)-2-(thio)uracil | — | U | NO |
| 5 (methoxycarbonyl-methyl)uracil | — | U | NO |
| 5 (methyl) 2 (thio)uracil | — | U | NO |
| 5 (methyl) 2,4 (dithio)uracil | — | U | NO |
| 5 (methyl) 4 (thio)uracil | — | U | NO |
| 5 (methylaminomethyl)-2 (thio)uracil | — | U | NO |
| 5 (methylaminomethyl)-2,4 (dithio)uracil | — | U | NO |
| 5 (methylaminomethyl)-4 (thio)uracil | — | U | NO |
| 5 (propynyl)uracil | — | U | NO |
| 5 (trifluoromethyl)uracil | — | U | NO |
| 5-(2-aminopropyl)uracil | — | U | NO |
| 5-(alky1)-2-(thio)pseudouracil | — | U | NO |
| 5-(alkyl)-2,4 (dithio)pseudouracil | — | U | NO |
| 5-(alky1)-4 (thio)pseudouracil | — | U | NO |
| 5-(alkyl)pseudouracil | — | U | NO |
| 5-(alkyl)uracil | — | U | NO |
| 5-(alkynyl)uracil | — | U | NO |
| 5-(allylamino)uracil | — | U | NO |
| 5-(cyanoalkyl)uracil | — | U | NO |
| 5-(dialkylaminoalkyl)uracil | — | U | NO |
| 5-(dimethylaminoalkyl)uracil | — | U | NO |
| 5-(guanidiniumalkyl)uracil | — | U | NO |
| 5-(halo)uracil | — | U | NO |
| 5-(1,3-diazole-l-alkyl)uracil | — | U | NO |
| 5-(methoxy)uracil | — | U | NO |
| 5-(methoxycarbonylmethyl)-2- | — | U | NO |
| (thio)uracil | | | |
| 5-(methoxycarbonyl-methyl)uracil | — | U | NO |
| 5-(methyl) 2(thio)uracil | — | U | NO |
| 5-(methyl) 2,4 (dithio)uracil | — | U | NO |
| 5-(methyl) 4 (thio)uracil | — | U | NO |
| 5-(methyl)-2-(thio)pseudouracil | — | U | NO |
| 5-(methyl)-2,4 (dithio)pseudouracil | — | U | NO |
| 5-(methyl)-4 (thio)pseudouracil | — | U | NO |
| 5-(methyl)pseudouracil | — | U | NO |
| 5-(methylaminomethyl)-2 (thio)uracil | — | U | NO |
| 5-(methylaminomethyl)-2,4(dithio)uracil | — | U | NO |
| 5-(methylaminomethyl)-4-(thio)uracil | — | U | NO |
| 5-(propyny1)uracil | — | U | NO |
| 5-(trifluoromethyl)uracil | — | U | NO |
| 5-aminoallyl-uridine | — | U | NO |
| 5-bromo-uridine | — | U | NO |
| 5-iodo-uridine | — | U | NO |
| 5-uracil | — | U | NO |
| 6 (azo)uracil | — | U | NO |
| 6-(azo)uracil | — | U | NO |
| 6-aza-uridine | — | U | NO |
| allyamino-uracil | — | U | NO |
| aza uracil | — | U | NO |
| deaza uracil | — | U | NO |
| N3 (methyl)uracil | — | U | NO |
| P seudo-UTP-1-2-ethanoic acid | — | U | NO |
| pseudouracil | — | U | NO |
| 4-Thio-pseudo-UTP | — | U | NO |
| 1-carboxymethyl-pseudouridine | — | U | NO |
| l-methyl-1-deaza-pseudouridine | — | U | NO |
| 1-propynyl-uridine | — | U | NO |
| l-taurinomethyl-1-methyl-uridine | — | U | NO |
| l-taurinomethyl-4-thio-uridine | — | U | NO |
| 1-taurinomethyl-pseudouridine | — | U | NO |
| 2-methoxy-4-thio-pseudouridine | — | U | NO |
| 2-thio-l-methyl-1-deaza-pseudouridine | — | U | NO |
| 2-thio-1-methyl-pseudouridine | — | U | NO |
| 2-thio-5-aza-uridine | — | U | NO |
| 2-thio-dihydropseudouridine | — | U | NO |
| 2-thio-dihydrouridine | — | U | NO |
| 2-thio-pseudouridine | — | U | NO |
| 4-methoxy-2-thio-pseudouridine | — | U | NO |
| 4-methoxy-pseudouridine | — | U | NO |
| 4-thio-1-methyl-pseudouridine | — | U | NO |
| 4-thio-pseudouridine | — | U | NO |
| 5-aza-uridine | — | U | NO |
| dihydropseudouridine | — | U | NO |
| (±)1-(2-Hydroxypropyl)pseudouridine TP | — | U | NO |
| (2R)-l-(2-Hydroxypropyl)pseudouridine | — | U | NO |
| TP | | | |
| (2S)-l-(2-Hydroxypropyl)pseudouridine | — | U | NO |
| TP | | | |
| (E)-5-(2-Bromo-vinyl)ara-uridine TP | — | U | NO |
| (E)-5-(2-Bromo-vinyl)uridine TP | — | U | NO |
| (Z)-5-(2-Bromo-vinyl)ara-uridine TP | — | U | NO |
| (Z)-5-(2-Bromo-vinyl)uridine TP | — | U | NO |
| 1-(2,2,2-Trifluoroethyl)-pseudo-UTP | — | U | NO |
| 1-(2,2,3,3,3- | — | U | NO |
| Pentafluoropropyl)pseudouridine TP | | | |
| 1-(2,2-Diethoxyethyl)pseudouridine TP | — | U | NO |
| 1-(2,4,6-Trimethylbenzyl)pseudouridine | — | U | NO |
| TP | | | |
| 1-(2,4,6-Trimethyl-benzyl)pseudo-UTP | — | U | NO |
| 1-(2,4,6-Trimethyl-phenyl)pseudo-UTP | — | U | NO |
| 1-(2-Amino-2-carboxyethyl)pseudo-UTP | — | U | NO |
| 1-(2-Amino-ethyl)pseudo-UTP | — | U | NO |
| 1-(2-Hydroxyethyl)pseudouridine TP | — | U | NO |
| 1-(2-Methoxyethyl)pseudouridine TP | — | U | NO |
| 1-(3,4-Bis- | — | U | NO |
| trifluoromethoxybenzvl)pseudouridine | | | |
| TP | | | |
| 1-(3,4-Dimethoxybenzyl)pseudouridine | — | U | NO |
| TP | | | |
| 1-(3-Amino-3-carboxypropyl)pseudo- | — | U | NO |
| UTP | | | |
| 1-(3-Amino-propyl)pseudo-UTP | — | U | NO |
| 1-(3-Cyclopropyl-prop-2- | — | U | NO |
| ynyl)pseudouridine TP | | | |
| 1-(4-Amino-4-carboxybutyl)pseudo-UTP | — | U | NO |
| 1-(4-Amino-benzyl)pseudo-UTP | — | U | NO |
| 1-(4-Amino-buty l)pseudo-UTP | — | U | NO |
| 1-(4-Amino-phenyl)pseudo-UTP | — | U | NO |
| 1-(4-Azidobenzyl)pseudouridine TP | — | U | NO |
| 1-(4-Bromobenzyl)pseudouridine TP | — | U | NO |
| 1-(4-Chlorobenzyl)pseudouridine TP | — | U | NO |
| 1-(4-Fluorobenzyl)pseudouridine TP | — | U | NO |
| 1-(4-Iodobenzyl)pseudouridine TP | — | U | NO |
| 1-(4- | — | U | NO |
| Methanesulfonylbenzyl)pseudouridine TP | | | |
| 1-(4-Methoxybenzyl)pseudouridine TP | — | U | NO |
| 1-(4-Methoxy-benzy l)pseudo-UTP | — | U | NO |
| 1-(4-Methoxy-phenyl)pseudo-UTP | — | U | NO |
| 1-(4-Methylbenzyl)pseudouridine TP | — | U | NO |
| 1-(4-Methyl-benzyl)pseudo-UTP | — | U | NO |
| 1-(4-Nitrobenzyl)pseudouridine TP | — | U | NO |
| 1-(4-Nitro-benzyl)pseudo-UTP | — | U | NO |
| 1(4-Nitro-phenyl)pseudo-UTP | — | U | NO |
| 1-(4-Thiomethoxybenzyl)pseudouridine | — | U | NO |
| TP | | | |
| 1-(4- | — | U | NO |
| Trifluoromethoxybenzyl)pseudouridine | | | |
| TP | | | |
| 1-(4- | — | U | NO |
| Trifluoromethylbenzyl)pseudouridine TP | | | |
| 1-(5-Amino-pentyl)pseudo-UTP | — | U | NO |
| 1-(6-Amino-hexyl)pseudo-UTP | — | U | NO |
| 1,6-Dimethyl-pseudo-UTP | — | U | NO |
| 1-[3-(2-{2-[2-(2-Aminoethoxy)-ethoxy]- | — | U | NO |
| ethoxy}-ethoxy)-propionyl]pseudouridine | | | |
| TP | | | |
| 1-{3-[2-(2- Aminoethoxy)-ethoxy]- | — | U | NO |
| propionvl} pseudouridine TP | | | |
| 1-Acetylpseudouridine TP | — | U | NO |
| l-Alkyl-6-(1-propynyl)-pseudo-UTP | — | U | NO |
| l-Alkyl-6-(2-propynyl)-pseudo-UTP | — | U | NO |
| l-Alkyl-6-allyl-pseudo-UTP | — | U | NO |
| l-Alkyl-6-ethynyl-pseudo-UTP | — | U | NO |
| l-Alkyl-6-homoallyl-pseudo-UTP | — | U | NO |
| l-Alkyl-6-vinyl-pseudo-UTP | — | U | NO |
| 1-Allylpseudouridine TP | — | U | NO |
| 1-Aminomethyl-pseudo-UTP | — | U | NO |
| 1-Benzoylpseudouridine TP | — | U | NO |
| 1-Benzyloxymethylpseudouridine TP | — | U | NO |
| 1-Benzyl-pseudo-UTP | — | U | NO |
| l-Biotinyl-PEG2-pseudouridine TP | — | U | NO |
| 1-Biotinylpseudouridine TP | — | U | NO |
| 1-Butyl-pseudo-UTP | — | U | NO |
| 1-Cyanomethylpseudouridine TP | — | U | NO |
| 1-Cyclobutylmethyl-pseudo-UTP | — | U | NO |
| 1-Cyclobutyl-pseudo-UTP | — | U | NO |
| 1-Cycloheptylmethyl-pseudo-UTP | — | U | NO |
| 1-Cycloheptyl-pseudo-UTP | — | U | NO |
| 1-Cyclohexylmethyl-pseudo-UTP | — | U | NO |
| 1-Cyclohexyl-pseudo-UTP | — | U | NO |
| 1-Cyclooctylmethyl-pseudo-UTP | — | U | NO |
| 1-Cyclooctyl-pseudo-UTP | — | U | NO |
| 1-Cyclopentylmethyl-pseudo-UTP | — | U | NO |
| 1-Cyclopentyl-pseudo-UTP | — | U | NO |
| 1-Cyclopropylmethyl-pseudo-UTP | — | U | NO |
| 1-Cyclopropyl-pseudo-UTP | — | U | NO |
| 1-Ethyl-pseudo-UTP | — | U | NO |
| 1-Hexyl-pseudo-UTP | — | U | NO |
| 1-Homoallylpseudouridine TP | — | U | NO |
| 1-Hydroxymethylpseudouridine TP | — | U | NO |
| 1-iso-propyl-pseudo-UTP | — | U | NO |
| 1-Me-2-thio-pseudo-UTP | — | U | NO |
| 1-Me-4-thio-pseudo-UTP | — | U | NO |
| 1-Me-alpha-thio-pseudo-UTP | — | U | NO |
| 1-Methanesulfonylmethylpseudouridine | — | U | NO |
| TP | | | |
| 1-Methoxymethylpseudouridine TP | — | U | NO |
| l-Methyl-6-(2,2,2-Trifluoroethyl)pseudo- | — | U | NO |
| UTP | | | |
| l-Methyl-6-(4-morpholino)-pseudo-DTP | — | U | NO |
| l-Methyl-6-(4-thiomorpholino)-pseudo- | — | U | NO |
| UTP | | | |
| l-Methyl-6-(substituted phenyl)pseudo- | — | U | NO |
| UTP | | | |
| 1-Methyl-6-amino-pseudo-UTP | — | U | NO |
| l-Methyl-6-azido-pseudo-UTP | — | U | NO |
| 1-Methyl-6-bromo-pseudo-UTP | — | U | NO |
| l-Methyl-6-butyl-pseudo-UTP | — | U | NO |
| l-Methyl-6-chloro-pseudo-UTP | — | U | NO |
| 1-Methyl-6-cyano-pseudo-UTP | — | U | NO |
| l-Methyl-6-dimethylamino-pseudo-UTP | — | U | NO |
| l-Methyl-6-ethoxy-pseudo-UTP | — | U | NO |
| l-Methyl-6-ethylcarboxylate-pseudo- | — | U | NO |
| UTP | | | |
| l-Methyl-6-ethyl-pseudo-UTP | — | U | NO |
| l-Methyl-6-fluoro-pseudo-UTP | — | U | NO |
| l-Methyl-6-formyl-pseudo-UTP | — | U | NO |
| 1-Methyl-6-hydroxyamino-pseudo-UTP | — | U | NO |
| l-Methyl-6-hydroxy-pseudo-UTP | — | U | NO |
| l-Methyl-6-iodo-pseudo-UTP | — | U | NO |
| l-Methyl-6-iso-propyl-pseudo-UTP | — | U | NO |
| l-Methyl-6-methoxy-pseudo-UTP | — | U | NO |
| l-Methyl-6-methylamino-pseudo-UTP | — | U | NO |
| l-Methyl-6-phenyl-pseudo-UTP | — | U | NO |
| l-Methyl-6-propyl-pseudo-UTP | — | U | NO |
| l-Methyl-6-tert-butyl-pseudo-UTP | — | U | NO |
| 1-Methyl-6-trifluoromethoxy-pseudo- | — | U | NO |
| UTP | | | |
| l-Methyl-6-trifluoromethyl-pseudo-UTP | — | U | NO |
| 1-Morpholinomethylpseudouridine TP | — | U | NO |
| 1-Pentyl-pseudo-UTP | — | U | NO |
| 1-Phenyl-pseudo-UTP | — | U | NO |
| 1-Piyaloylpseudouridine TP | — | U | NO |
| 1-Propargylpseudouridine TP | — | U | NO |
| 1-Propyl-pseudo-UTP | — | U | NO |
| 1-propynyl-pseudouridine | — | U | NO |
| 1-p-tolyl-pseudo-UTP | — | U | NO |
| 1-tert-Butyl-pseudo-UTP | — | U | NO |
| 1-Thiomethoxymethylpseudouridine TP | — | U | NO |
| 1-Thiomorpholinomethylpseudouridine | — | U | NO |
| TP | | | |
| 1-Trifluoroacetylpseudouridine TP | — | U | NO |
| 1-Trifluoromethyl-pseudo-UTP | — | U | NO |
| 1-Vinylpseudouridine TP | — | U | NO |
| 2,2′-anhydro-uridine TP | — | U | NO |
| 2′-bromo-deoxyuridine TP | — | U | NO |
| 2′-F-5-Methyl-2′-deoxy-UTP | — | U | NO |
| 2′-OMe-5-Me-UTP | — | U | NO |
| 2′-OMe-pseudo-UTP | — | U | NO |
| 2′-a-Ethynyluridine TP | — | U | NO |
| 2′-a-Trifluoromethyluridine TP | — | U | NO |
| 2′-b-Ethynyluridine TP | — | U | NO |
| 2′-b-Trifluoromethyluridine TP | — | U | NO |
| 2′-Deoxy-2′,2′-difluorouridine TP | — | U | NO |
| 2′-Deoxy-2′-a-mercaptouridine TP | — | U | NO |
| 2′-Deoxy-2′-a-thiomethoxyuridine TP | — | U | NO |
| 2′-Deoxy-2′-b-aminouridine TP | — | U | NO |
| 2′-Deoxy-2′-b-azidouridine TP | — | U | NO |
| 2′-Deoxy-2′-b-bromouridine TP | — | U | NO |
| 2′-Deoxy-2′-b-chlorouridine TP | — | U | NO |
| 2′-Deoxy-2′-b-fluorouridine TP | — | U | NO |
| 2′-Deoxy-2′-b-iodouridine TP | — | U | NO |
| 2′-Deoxy-2′-b-mercaptouridine TP | — | U | NO |
| 2′-Deoxy-2′-b-thiomethoxyuridine TP | — | U | NO |
| 2-methoxy-4-thio-uridine | — | U | NO |
| 2-methoxyuridine | — | U | NO |
| 2′-O-Methyl-5-(1-propynyl)uridine TP | — | U | NO |
| 3-Alkyl-pseudo-UTP | — | U | NO |
| 4′-Azidouridine TP | — | U | NO |
| 4′-Carbocyclic uridine TP | — | U | NO |
| 4′-Ethynyluridine TP | — | U | NO |
| 5-(1-Propynyl)ara-uridine TP | — | U | NO |
| 5-(2-Furanyl)uridine TP | — | U | NO |
| 5-Cyanouridine TP | — | U | NO |
| 5-Dimethylaminouridine TP | — | U | NO |
| 5′-Homo-uridine TP | — | U | NO |
| 5-iodo-2′-fluoro-deoxyuridine TP | — | U | NO |
| 5-Phenylethynyluridine TP | — | U | NO |
| 5-Trideuteromethyl-6-deuterouridine TP | — | U | NO |
| 5-Trifluoromethyl-Uridine TP | — | U | NO |
| 5-Vinylarauridine TP | — | U | NO |
| 6-(2,2,2-Trifluoroethyl)-pseudo-UTP | — | U | NO |
| 6-(4-Morpholino)-pseudo-DTP | — | U | NO |
| 6-(4-Thiomorpholino)-pseudo-UTP | — | U | NO |
| 6-(Substituted-Phenyl)-pseudo-UTP | — | U | NO |
| 6-Amino-pseudo-UTP | — | U | NO |
| 6-Azido-pseudo-UTP | — | U | NO |
| 6-Bromo-pseudo-UTP | — | U | NO |
| 6-Butyl-pseudo-UTP | — | U | NO |
| 6-Chloro-pseudo-UTP | — | U | NO |
| 6-Cyano-pseudo-UTP | — | U | NO |
| 6-Dimethylamino-pseudo-UTP | — | U | NO |
| 6-Ethoxy-pseudo-UTP | — | U | NO |
| 6-Ethylcarboxylate-pseudo-UTP | — | U | NO |
| 6-Ethyl-pseudo-UTP | — | U | NO |
| 6-Fluoro-pseudo-UTP | — | U | NO |
| 6-Formyl-pseudo-UTP | — | U | NO |
| 6-Hydroxyamino-pseudo-UTP | — | U | NO |
| 6-Hydroxy-pseudo-UTP | — | U | NO |
| 6-Iodo-pseudo-UTP | — | U | NO |
| 6-iso-Propyl-pseudo-UTP | — | U | NO |
| 6-Methoxy-pseudo-UTP | — | U | NO |
| 6-Methylamino-pseudo-UTP | — | U | NO |
| 6-Methyl-pseudo-UTP | — | U | NO |
| 6-Phenyl-pseudo-UTP | — | U | NO |
| 6-Phenyl-pseudo-UTP | — | U | NO |
| 6-Propyl-pseudo-UTP | — | U | NO |
| 6-tert-Butyl-pseudo-UTP | — | U | NO |
| 6-Trifluoromethoxy-pseudo-UTP | — | U | NO |
| 6-Trifluoromethyl-pseudo-UTP | — | U | NO |
| Alpha-thio-pseudo-UTP | — | U | NO |
| Pseudouridine 1-(4- | — | U | NO |
| methylbenzenesulfonic | | | |
| acid) TP | | | |
| Pseudouridine 1-(4-methylbenzoic acid) | — | U | NO |
| TP | | | |
| Pseudouridine TP l-[3-(2- | — | U | NO |
| ethoxy)]propionic acid | | | |
| Pseudouridine TP l-[3-{2-(2-[2-(2-ethoxy)- | — | U | NO |
| ethoxy]-ethoxy)-ethoxy}]propionic | | | |
| acid | | | |
| Pseudouridine TP l-[3-{2-(2-[2-{2(2- | — | U | NO |
| ethoxy)-ethoxy}-ethoxy]-ethoxy)- | | | |
| ethoxy}]propionic acid | | | |
| Pseudouridine TP l-[3-{2-(2-[2-ethoxy]- | — | U | NO |
| ethoxy)-ethoxv}]propionic acid | | | |
| Pseudouridine TP l-[3-{2-(2-ethoxy)- | — | U | NO |
| ethoxv}] propionic acid | | | |
| Pseudouridine TP 1-methylphosphonic | — | U | NO |
| acid | | | |
| Pseudouridine TP 1-methylphosphonic | — | U | NO |
| acid diethyl ester | | | |
| Pseudo-UTP-N1-3-propionic acid | — | U | NO |
| Pseudo-UTP-N1-4-butanoic acid | — | U | NO |
| Pseudo-UTP-N 1-5-pentanoic acid | — | U | NO |
| Pseudo-UTP-N1-6-hexanoic acid | — | U | NO |
| Pseudo-UTP-Nl-7-heptanoic acid | — | U | NO |
| Pseudo-UTP-N1-methy1-p-benzoic acid | — | U | NO |
| Pseudo-UTP-N1-p-benzoic acid | — | U | NO |

In some embodiments, a genetic element described herein comprises a modification provided in Table 6, or a combination thereof. The modifications provided in Table 6 occur naturally in RNAs, and may be used herein in a genetic element at a position that does not occur in nature.

TABLE 6
Additional exemplary modifications

| | | | Natu- |
|---|---|---|---|
| | | | rally |
| | | | Oc- |
| Name | Symbol | Base | curring |
| 2-methylthio-N6-(cis- | ms2i6A | A | YES |
| hvdroxvisopentenvl)adenosine | | | |
| 2-methylthio-N6-methyladenosine | ms2m6A | A | YES |
| 2-methylthio-N6-threonyl | ms2t6A | A | YES |
| carbamoyladenosine | | | |
| N6-glycinylcarbamoyladenosine | g6A | A | YES |
| N6-isopentenyladenosine | i6A | A | YES |
| N6-methyladenosine | m6A | A | YES |
| N6-threonylcarbamoyladenosine | t6A | A | YES |
| 1,2′-O-dimethyladenosine | mlAm | A | YES |
| 1-methyladenosine | mlA | A | YES |
| 2′-O-methyladenosine | Am | A | YES |
| 2′-O-ribosyladenosine (phosphate) | Ar(p) | A | YES |
| 2-methyladenosine | m2A | A | YES |
| 2-methylthio-N6 isopentenyladenosine | ms2i6A | A | YES |
| 2-methylthio-N6-hydroxynorvalyl | ms2hn6A | A | YES |
| carbamoyladenosine | | | |
| 2′-O-methyladenosine | m6A | A | YES |
| 2′-O-ribosyladenosine (phosphate) | Ar(p) | A | YES |
| isopenteny ladenosine | Iga | A | YES |
| N6-(cis-hydroxyisopentenyl)adenosine | io6A | A | YES |
| N6,2′-O-dimethyladenosine | m6Am | A | YES |
| N6,2′-O-dimethyladenosine | m⁶Am | A | YES |
| N6,N6,2′-O-trimethyladenosine | m62Am | A | YES |
| N6,N6-dimethyladenosine | m62A | A | YES |
| N6-acetyladenosine | ac6A | A | YES |
| N6-hydroxynorvalylcarbamoyladenosine | hn6A | A | YES |
| N6-methyl-N6- | m6t6A | A | YES |
| threonylcarbamoyladenosine | | | |
| 2-methyladenosine | m²A | A | YES |
| 2-methylthio-N⁶-isopenteny ladenosine | ms²i⁶A | A | YES |
| 2-thiocytidine | s2C | C | YES |
| 3-methylcytidine | m3C | C | YES |
| 5-formylcytidine | f5C | C | YES |
| 5-hydroxymethyl cytidine | hm5C | C | YES |
| 5-methylcytidine | m5C | C | YES |
| N4-acetylcytidine | ac4C | C | YES |
| 2′-O-methylcytidine | Cm | C | YES |
| 2′-O-methylcytidine | Cm | C | YES |
| 5,2′-O-dimethylcytidine | m5Cm | C | YES |
| 5-formyl-2′-O-methylcytidine | f5Cm | C | YES |
| lysidine | k2C | C | YES |
| N4,2′-O-dimethy lcytidine | m4Cm | C | YES |
| N4-acetyl-2′-O-methylcytidine | ac4Cm | C | YES |
| N4-methylcytidine | m4C | C | YES |
| N4,N4-Dimethyl-2′-OMe-Cytidine TP | — | C | YES |
| 7-methylguanosine | m7G | G | YES |
| N2,2′-O-dimethylguanosine | m2Gm | G | YES |
| N2-methylguanosine | m2G | G | YES |
| wyosine | imG | G | YES |
| 1,2′-O-dimethylguanosine | mlGm | G | YES |
| 1-methylguanosine | mlG | G | YES |
| 2′-O-methylguanosine | Gm | G | YES |
| 2′-O-ribosylguanosine (phosphate) | Gr(p) | G | YES |
| 2′-O-methylguanosine | Gm | G | YES |
| 2′-O-ribosylguanosine (phosphate) | Gr(p) | G | YES |
| 7-aminomethyl-7-deazaguanosine | preQl | G | YES |
| 7-cyano-7-deazaguanosine | preQ0 | G | YES |
| archaeosine | G+ | G | YES |
| methylwyosine | mimG | G | YES |
| N2,7-dimethylguanosine | m2, 7G | G | YES |
| N2,N2,2′-O-trimethylguanosine | m22Gm | G | YES |
| N2,N2,7-trimethylguanosine | m2, 2, 7G | G | YES |
| N2,N2-dimethylguanosine | m22G | G | YES |
| N2,7,2′-O-trimethylguanosine | m2 7 | G | YES |
| | ′Gm | | |
| 1-methylinosine | mll | A | YES |
| inosine | I | A | YES |
| 1,2′-O-dimethylinosine | mlim | A | YES |
| 2′-O-methylinosine | Im | A | YES |
| 2′-O-methylinosine | Im | A | YES |
| epoxyqueuosine | oQ | G | YES |
| galactosyl-queuosine | galQ | G | YES |
| mannosyl-queuosine | manQ | G | YES |
| 2′-O-methyluridine | — | U | YES |
| 2-thiouridine | s2U | U | YES |
| 3-methyluridine | m3U | U | YES |
| 5-carboxymethyluridine | cm5U | U | YES |
| 5-hydroxyuridine | ho5U | U | YES |
| 5-methyluridine | m5U | U | YES |
| 5-taurinomethyl-2-thiouridine | rm5s2U | U | YES |
| 5-taurinomethyluridine | rm5U | U | YES |
| dihydrouridine | D | U | YES |
| pseudouridine | Q | U | YES |
| (3-(3-amino-3-carboxypropyl)uridine | acp3U | U | YES |
| l-methyl-3-(3-amino-5- | mlacp3′P | U | YES |
| carboxypropyl)pseudouridine | | | |
| 1-methylpseduouridine | ml′P | U | YES |
| 1-methyl-pseudouridine | — | U | YES |
| 2′-0-methyluridine | Um | U | YES |
| 2′-0-methylpseudouridine | ′Pm | U | YES |
| 2′-0-methyluridine | Um | U | YES |
| 2-thio-2′-0-methyluridine | s2Um | U | YES |
| 3-(3-amino-3-carboxypropyl)uridine | acp3U | U | YES |
| 3,2′-0-dimethyluridine | m3Um | U | YES |
| 3-Methyl-pseudo-Uridine TP | — | U | YES |
| 4-thiouridine | s4U | U | YES |
| 5-(carboxyhydroxymethyl)uridine | chm5U | U | YES |
| 5-(carboxyhydroxymethyl)uridine methyl | mchm5U | U | YES |
| ester | | | |
| 5,2′-0-dimethyluridine | m5Um | U | YES |
| 5,6-dihydro-uridine | — | U | YES |
| 5-aminomethy1-2-thiouridine | nm5s2U | U | YES |
| 5-carbamoylmethyl-2′-0-methyluridine | ncm5Um | U | YES |
| 5-carbamoylmethyluridine | ncm5U | U | YES |
| 5-carboxyhydroxymethyluridine | — | U | YES |
| 5-carboxyhydroxymethyluridine methyl | — | U | YES |
| ester | | | |
| 5-carboxymethylaminomethyl-2′-0- | cmnm5Um | U | YES |
| methyluridine | | | |
| 5-carboxymethylaminomethyl-2- | cmnm5s2U | U | YES |
| thiouridine | | | |
| 5-carboxymethylaminomethyl-2- | — | U | YES |
| thiouridine | | | |
| 5-carboxymethylaminomethyluridine | cmnm5U | U | YES |
| 5-carboxymethylaminomethyluridine | — | U | YES |
| 5-Carbamoylmethyluridine TP | — | U | YES |
| 5-methoxycarbonylmethyl-2′-0- | mcm5Um | U | YES |
| methyluridine | | | |
| 5-methoxycarbonylmethy1-2-thiouridine | mcm5s2U | U | YES |
| 5-methoxycarbonylmethyluridine | mcm5U | U | YES |
| 5-methoxyuridine | mo5U | U | YES |
| 5-methyl-2-thiouridine | m5s2U | U | YES |
| 5-methylaminomethyl-2-selenouridine | mnm5se2U | U | YES |
| 5-methylaminomethyl-2-thiouridine | mnm5s2U | U | YES |
| 5-methylaminomethyluridine | mnm5U | U | YES |
| 5-Methyldihydrouridine | — | U | YES |
| 5-0xyacetic acid- Uridine TP | — | U | YES |
| 5-0xyacetic acid-methyl ester-Uridine TP | — | U | YES |
| Nl-methyl-pseudo-uridine | — | U | YES |
| uridine 5-oxyacetic acid | cmo5U | U | YES |
| uridine 5-oxyacetic acid methyl ester | mcmo5U | U | YES |
| 3-(3-Amino-3-carboxypropyl)-Uridine | — | U | YES |
| TP | | | |
| 5-(iso-Pentenylaminomethyl)-2- | — | U | YES |
| thiouridine TP | | | |
| 5-(iso-Pentenylaminomethyl)-2′-0- | — | U | YES |
| methyluridine TP | | | |
| 5-(iso-Pentenylaminomethyl)uridine TP | — | U | YES |
| wybutosine | yW | A/T | YES |
| hydroxywybutosine | OHyW | A/T | YES |
| isowyosine | imG2 | A/T | YES |
| peroxywybutosine | o2yW | A/T | YES |
| undermodified hydroxywybutosine | OHyW* | A/T | YES |
| 4-demethylwyosine | imG-14 | A/T | YES |

In an embodiment, a genetic element described herein comprises a non-naturally occurring modification provided in Table 7, or a combination thereof.

TABLE 7
Additional exemplary non-naturally occurring modifications
Name

2,6-(diamino)purine
1-(aza)-2-(thio)-3-(aza)-phenoxazin-1-yl
1,3-(diaza)-2-(oxo)-phenthiazin-1-yl
1,3-(diaza)-2-(oxo)-phenoxazin-1-yl
1,3,5-(triaza)-2,6-(dioxa)-naphthalene
2 (amino)purine
2,4,5-(trimethyl)phenyl
2′methyl, 2′amino, 2′azido, 2′fluro-cytidine
2′methyl, 2′amino, 2′azido, 2′fluro-adenine
2′methyl, 2′amino, 2′azido, 2′fluro-uridine
2′-amino-2′-deoxyribose
2-amino-6-Chloro-purine
2-aza-inosinyl
2′-azido-2′-deoxyribose
2′fluoro-2′-deoxyribose
2′-fluoro-modified bases
2′-O-methyl-ribose
2-oxo-7-aminopyridopyrimidin-3-yl
2-oxo-pyridopyrimidine-3 -yl
2-pyridinone
3 nitropyrrole
3-(methyl)-7-(propynyl)isocarbostyrilyl
3-(methyl)isocarbostyrilyl
4-(fluoro)-6-(methyl)benzimidazole
4-(methyl)benzimidazole
4-(methyl)indolyl
4,6-(dimethyl)indolyl
5 nitroindole
5 substituted pyrimidines
5-(methyl)isocarbostyrilyl
5-nitroindole
6-(aza)pyrimidine
6-(azo)thymine
6-(methyl)-7-(aza)indolyl
6-chloro-purine
6-phenyl-pyrrolo-pyrimidin-2-on-3-yl
7-(aminoalkylhydroxy)-1-(aza)-2-(thio)-3-(aza)-
phenthiazin-1-yl
7-(aminoalkylhydroxy)-1-(aza)-2-(thio)-3-(aza)-
phenoxazin-1-yl
7-(aminoalkylhydroxy)-1,3-(diaza)-2-(oxo)-phenoxazin-1-
yl
7-(aminoalkylhydroxy)-1,3-(diaza)-2-(oxo)-phenthiazin-
1-yl
7-(aminoalkylhydroxy)-1,3-(diaza)-2-(oxo)-phenoxazin-l-
yl
7-(aza)indolyl
7-(guanidiniumalkylhydroxy)-1-(aza)-2-(thio)-3-(aza)-
phenoxazinl-yl
7-(guanidiniumalkylhydroxy)-1-(aza)-2-(thio)-3-(aza)-
phenthiazin-1-yl
7-(guanidiniumalkylhydroxy)-1-(aza)-2-(thio)-3-(aza)-
phenoxazin-1-yl
7-(guanidiniumalkylhydroxy)-1,3-(diaza)-2-(oxo)-
phenoxazin-1-yl
7-(guanidiniumalkyl-hydroxy)-1,3-(diaza)-2-(oxo)-
phenthiazin-1-yl
7-(guanidiniumalkylhydroxy)-1,3-(diaza)-2-(oxo)-
phenoxazin-1-yl
7-(propynyl)isocarbostyrilyl
7-(propynyl)isocarbostyrilyl, propynyl-7-(aza)indolyl
7-deaza-inosinyl
7-substituted 1-(aza)-2-(thio)-3-(aza)-phenoxazin-1-yl
7-substituted 1,3-(diaza)-2-(oxo)-phenoxazin-1-yl
9-(methyl)-imidizopyridinyl
aminoindolyl
anthracenyl
bis-ortho-(aminoalkylhydroxy)-6-phenyl-pyrrolo-
nvrimidin-2-on-3-yl
bis-ortho-substituted-6-phenyl-pyrrolo-pyrimidin-2-on-3-
yl
difluorotolyl
hypoxanthine
imidizopyridinyl
inosinyl
isocarbostyrilyl
isoguanine
N2-substituted purines
N6-methyl-2-amino-purine
N6-substituted purines
N-alkylated derivative
napthalenyl
nitrobenzimidazolyl
nitroimidazolyl
nitroindazolyl
nitropyrazolyl
nubularine
O6-substituted purines
O-alkylated derivative
ortho-(aminoalkylhydroxy)-6-phenyl-pyrrolo-pyrimidin-2-
on-3-yl
ortho-substituted-6-phenyl-pyrrolo-pyrimidin-2-on-3-yl
Oxoformycin TP
para-(aminoalkylhydroxy)-6-phenyl-pyrrolo-pyrimidin-2-
on-3-yl
para-substituted-6-phenyl-pyrrolo-pyrimidin-2-on-3-yl
pentacenyl
phenanthracenyl
phenyl
propynyl-7-(aza)indolyl
pyrenyl
pyridopyrimidin-3-yl
pyridopyrimidin-3-yl, 2-oxo-7-amino-pyridopyrimidin-3-
yl
pyrrolo-pyrimidin-2-on-3-yl
pyrrolopyrimidinyl
pyrrolopyrizinyl
stilbenzyl
substituted 1,2,4-triazoles
tetracenyl
tubercidine
xanthine
Xanthosine-5′-TP
2-thio-zebularine
5-aza-2-thio-zebularine
7-deaza-2-amino-purine
pyridin-4-one ribonucleoside
2-Amino-riboside-TP
Formycin A TP
Formycin B TP
Pyrrolosine TP
2′-OH-ara-adenosine TP
2′-OH-ara-cytidine TP
2′-OH-ara-uridine TP
2′-OH-ara-guanosine TP
5-(2-carbomethoxyvinyl)uridine TP
N6-(19-Amino-pentaoxanonadecyl)adenosine TP

In an embodiment, a genetic element described herein comprises a non-naturally occurring modification provided in Table 8, or a combination thereof.

TABLE 8
Exemplary backbone modifications
Name

3′-alkylene phosphonates
3′-amino phosphoramidate
alkene containing backbones
aminoalkylphosphoramidates
aminoalkylphosphotriesters
boranophosphates
—CH2-0-N(CH3)—CH2—
—CH2—N(CH3)—N(CH3)—CH2—
—CH2—NH—CH2—
chiral phosphonates
chiral phosphorothioates
formacetyl and thioformacetyl backbones
methylene (methylimino)
methylene formacetyl and thioformacetyl backbones
methyleneimino and methylenehydrazino backbones
morpholino linkages
—N(CH3)—CH2—CH2—
oligonucleosides with heteroatom intemucleoside linkage
phosphinates
phosphoramidates
phosphorodithioates
phosphorothioate intemucleoside linkages
phosphorothioates
phosphotriesters
PNA
siloxane backbones
sulfamate backbones
sulfide sulfoxide and sulfone backbones
sulfonate and sulfonamide backbones
thionoalkylphosphonates
thionoalkylphosphotriesters
thionophosphoramidates

In an embodiment, a genetic element described herein comprises a non-naturally occurring modification provided in Table 9, or a combination thereof.

TABLE 9
Exemplary non-naturally occurring backbone modificiations
Name of synthetic backbone modifications

Phosphorothioate
Constrained nucleic acid (CNA)
2′O′methylation
2′-O-methoxyethylribose (MOE)
2′Fluoro
Locked nucleic acid (LNA)
(S)-constraincd ethyl (cEt)
Fluoro hexitol nucleic acid (FHNA)
5′phosphorothioate
Phosphorodiamidate Morpholino Oligomer (PMO)
Tricyclo-DNA (tcDNA)
(S) 5′-C-methyl
(E)-vinylphosphonate
Methyl phosphonate
(S) 5′-C-methyl with phosphate

In some embodiments, the genetic element comprises a cap. A cap is typically placed at the 5′ end of an mRNA, but a cap can also be positioned at the 3′ end of an RNA. In some embodiments, a cap protects the genetic element from exonuclease degradation, and can help in delivery and/or localization within a cell. The cap can be present at the 5′-terminus (5′-cap) or at the 3′-terminal (3′-cap) or can be present on both termini. Non-limiting examples of a 5′-cap include, but are not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′, 5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

In some embodiments, the genetic element comprises a poly-A tail. In some embodiments, a poly-A tail comprises at least about 5, 10, 15, 20, 25, 30, 40, 50, 60, 70, 80, 90, or 100 adenosines in length. In some embodiments, RNA lacks a poly-A tail. In some embodiments, wherein the RNA lacks a poly-A tail, the RNA comprises no more than about 5, 10, 15, 20, 25, 30, 40, 50, 60, 70, 80, 90, or 100 sequential adenosines.

In some embodiments, a genetic element is linear. In some embodiments, a genetic element is circular. In some embodiments, a genetic element comprises a first region and a second region that can hybridize with the first region. In some embodiments, a genetic element comprises a first region and a second region that can hybridize with the first region to form a circle. In some embodiments, a genetic element does not comprise a 5′ or a 3′ end. In some embodiments, a genetic element does not comprise one or both of a free phosphate and a free sugar. In some embodiments, every phosphate in a genetic element is covalently linked to a first sugar by a first oxygen atom comprised by the phosphate and a second sugar by a second oxygen atom comprised by a phosphate. In some embodiments, every sugar in a genetic element is covalently linked to a first phosphate by a first carbon atom comprised by the sugar and a second phosphate by a second carbon atom comprised by the sugar. In some embodiments, a genetic element is produced by circularizing a linear RNA. Circular RNAs are described, e.g., in US Patent Publication 20200306286, which is herein incorporated by reference in its entirety.

In some embodiments, a genetic element is about 10-20, 20-30, 30-40, 50-60 60-70, 70-80, 80-90, 90-100, 100-125, 125-150, 150-200, 200-300, 300-400, 400-500, 500-600, 600-700, 700-800, 800-900, 900-1000, 1000-1500, 1500-2000, 2000-2500, 2500-3000, 3000-3500, 3500-4000, or 4000-4500 nucleotides in length. In some embodiments a genetic element is at least about 10, 20, 30, 40, 50, 60, 70, 80, 90, 100, 125, 150, 175, 200, 250, 300, 400, 500, 600, 700, 800, 900, 1000, 1500, 2000, 2500, 3000, 3500, 4000, or 4500 nucleotides in length.

RNA-Only Genetic Elements

In some embodiments, a genetic element consists of or consists essentially of RNA. For example, in some embodiments, a genetic element is substantially free of DNA. In some embodiments, a genetic element comprises single stranded RNA. In some embodiments, a genetic element comprises at least one double stranded region. In some embodiments, a double stranded region of a genetic element comprises a region of RNA pairing with RNA.

Hybridized RNA-ssDNA Genetic Elements

In some embodiments, a genetic element comprises a DNA region. In some embodiments, a genetic element comprising RNA further comprises a DNA region. For example, a genetic element may be single stranded, wherein a first portion of the single strand comprises ribonucleotides and a second portion of the single strand comprises deoxyribonucleotides. In some embodiments, a genetic element comprising a DNA region comprises one or more DNA nucleotides with chemical modification. In some embodiments, a genetic element comprises a DNA region, wherein all nucleotides of the DNA region are chemically modified.

In some embodiments, at least a portion of a genetic element is single stranded. In some embodiments, a genetic element is single stranded. In some embodiments, a genetic element comprises ssDNA. In some embodiments, a genetic element comprises a double stranded region. In some embodiments, a double stranded region of a genetic element comprises a region of RNA pairing with RNA. In some such embodiments, a double stranded region of a genetic element comprises a region of DNA pairing with RNA. In some embodiments, at least a portion of the DNA region hybridizes to at least a portion of the RNA of the genetic element.

In some embodiments, a DNA region is about 10-20, 20-30, 30-40, 40-50, 50-60, 60-70, 70-80, 80-90, 90-100, 100-150, 150-200, 200-300, 300-400, 400-500, 500-600, 600-700, 700-800, 800-900, or 900-1000 nucleotides in length.

RNA/DNA Conjugates

In some embodiments, a genetic element comprises a DNA region. In some embodiments, a genetic element comprising RNA further comprises a DNA region. In some embodiments, a genetic element comprising a DNA region comprises one or more DNA nucleotides with chemical modification. In some embodiments, a genetic element comprising a DNA region, wherein all nucleotides of the DNA region are chemically modified.

In some embodiments, at least a portion of a genetic element is single stranded. In some embodiments, a genetic element is single stranded. In some embodiments, a genetic element comprises ssDNA. In some embodiments, a genetic element comprises a double stranded region. In some embodiments, a double stranded region of a genetic element comprises a region of RNA pairing with RNA. In some such embodiments, a double stranded region of a genetic element comprises a region of DNA pairing with RNA. In some embodiments, wherein a genetic element comprises RNA, a DNA region is covalently linked to the RNA of the genetic element.

In some embodiments, a DNA region is about 10-20, 20-30, 30-40, 40-50, 50-60, 60-70, 70-80, 80-90, 90-100, 100-150, 150-200, 200-300, 300-400, 400-500, 500-600, 600-700, 700-800, 800-900, or 900-1000 nucleotides in length.

### Genetic Element Constructs

In some embodiments, a genetic element is produced from a genetic element construct. For instance, in some embodiments, the genetic element construct is DNA, e.g., double stranded DNA, and the genetic element may be produced by transcription, generating an RNA genetic element.

The genetic element of an anellovector as described herein may be produced from a genetic element construct that comprises a genetic element region and optionally other sequence such as a bacmid (e.g., comprising a baculovirus genome or a fragment thereof, e.g., one or more baculovirus elements) or donor vector backbone. In some embodiments, the genetic element construct comprises an Anellovirus 5′ UTR (e.g., as described herein). A genetic element construct may be any nucleic acid construct suitable for delivery of the sequence of the genetic element into a host cell or cell-free system in which the genetic element can be enclosed within a proteinaceous exterior. In some embodiments, the genetic element construct comprises a promoter. In some embodiments, transcription from the genetic element construct produces an RNA genetic element.

In some embodiments, the genetic element construct is a linear nucleic acid molecule. In some embodiments, the genetic element construct is a circular nucleic acid molecule (e.g., a plasmid, bacmid, donor vector, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp.

Small Circular Nucleic Acid Constructs

In some embodiments, the genetic element construct is a circular nucleic acid construct, e.g., lacking a vector backbone (e.g., lacking a bacterial origin of replication and/or selectable marker). In embodiments, the genetic element is a single- or double-stranded circular nucleic acid construct. In embodiments, the circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid constructis between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

Cis/Trans Constructs

In some embodiments, a genetic element construct (e.g., a bacmid or donor vector) as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells).

In some embodiments, the genetic element construct (e.g., bacmid or donor vector) comprises a sequence encoding an Anellovirus ORF1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

Expression Cassettes

In some embodiments, a genetic element construct (e.g., bacmid or donor vector) comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

### Production of RNA-Based Genetic Elements

An RNA-based genetic element may be produced by a variety of methods. For example, a genetic element construct comprising DNA can be transcribed to produce a genetic element that comprises RNA, e.g., as described above. The transcription may take place, e.g., in a cell or a cell-free system. RNA may be synthesized in vitro, for example, by solid phase synthesis.

### Production of Protein Components

Protein components of an anellovector, e.g., ORF1, can be produced in a variety of ways described herein.

Baculovirus Expression Systems

A viral expression system, e.g., a baculovirus expression system, may be used to express proteins (e.g., for production of anellovectors), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF molecule, e.g., ORF1, ORF2, ORF2/2, ORF2/3, ORF1/1, or ORF1/2, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an alt site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculovirual vector comprises one or more non-baculoviral promoters, e.g., a mammalian promoter or an Anellovirus promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoter suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include ie1, ie2, ie0, et1, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH10BAC® competent cells, containing the bacmid DNA with a mini-attTn7 target site. In some embodiments, the baculovirus expression vector, e.g., pFASTBAC® vector, may have a promoter, e.g., a dual promoter (e.g., polyhedrin promoter, p10 promoter). Commercially available pFASTBAC® donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector. In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria, e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

Insect Cell Systems

The proteins described herein may be expressed in insect cells infected or transfected with recombinant baculovirus or bacmid DNA, e.g., as described above. In some embodiments, insect cells include: the Sf9 and Sf21 cells derived from Spodoptera frugiperda and the Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodoptera frugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolayer at a temperature between 25° to 30° C. with aeration. Additional information on insect cells is discussed, for example, in U.S. patent application Ser. Nos. 14/564,512 and 14/775,154, each of which is hereby incorporated by reference.

Mammalian Cell Systems

In some embodiments, the proteins described herein may be expressed in vitro in animal cell lines infected or transfected with a vector encoding the protein, e.g., as described herein. Animal cell lines envisaged in the context of the present disclosure include porcine cell lines, e.g., immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines suitable for expressing the proteins described herein include, but are not limited to cell lines of human or primate origin, such as human or primate kidney carcinoma cell lines.

### Effectors

The compositions and methods described herein can be used to produce a genetic element of an anellovector comprising a sequence encoding an effector (e.g., an exogenous effector or an endogenous effector), e.g., as described herein. In some embodiments, the genetic element is the effector, e.g., the genetic element is a functional RNA. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In Vitro Assembly Methods

An anellovector may be produced, e.g., by in vitro assembly. In some embodiments, the genetic element is contacted to ORF1 in vitro under conditions that allow for assembly.

In some embodiments, baculovirus constructs are used to produce Anellovirus proteins. These proteins may then be used, e.g., for in vitro assembly to encapsidate a genetic element, e.g., a genetic element comprising RNA. In some embodiments, a polynucleotide encoding one or more Anellovirus protein is fused to a promoter for expression in a host cell, e.g., an insect or animal cell. In some embodiments, the polynucleotide is cloned into a baculovirus expression system. In some embodiments, a host cell, e.g., an insect cell is infected with the baculovirus expression system and incubated for a period of time. In some embodiments, an infected cell is incubated for about 1, 2, 3, 4, 5, 10, 15, or 20 days. In some embodiments, an infected cell is lysed to recover the Anellovirus protein.

In some embodiments, an isolated Anellovirus protein is purified. In some embodiments, an Anellovirus protein is purified using purification techniques including but not limited to chelating purification, heparin purification, gradient sedimentation purification and/or SEC purification. In some embodiments, a purified Anellovirus protein is mixed with a genetic element to encapsidate the genetic element, e.g., a genetic element comprising RNA. In some embodiments, a genetic element is encapsidated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus (AV) ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding AV ORF1 may be untagged. In some embodiments, DNA encoding AV ORF1 may contain tags fused N-terminally and/or C-terminally. In some embodiments, DNA encoding AV ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding AV ORF1 may be expressed alone or in combination with any number of helper proteins. In some embodiments, DNA encoding AV ORF1 is expressed in combination with AV ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another AV ORF1 protein (e.g., jellyroll fragments or the C-terminal portion of Ring 2 ORF1 replaced with comparable portions of Ring 9 ORF1).

In some embodiments, the present disclosure describes a method of making an anellovector, the method comprising: (a) providing a mixture comprising: (i) a genetic element comprising RNA, and (ii) an ORF1 molecule; and (b) incubating the mixture under conditions suitable for enclosing the genetic element within a proteinaceous exterior comprising the ORF1 molecule, thereby making an anellovector; optionally wherein the mixture is not comprised in a cell. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

### Enrichment and Purification

Harvested anellovectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the harvested anellovectors are purified by affinity purification (e.g., heparin affinity purification). In some embodiments, the harvested anellovectors are purified by size exclusion chromatography (e.g., using a Tris buffer mobile phase). In some embodiments, the harvested anellovectors are purified by anion exchange chromatography (e.g., Mustang Q membrane chromatography). In some embodiments, the harvested anellovectors are purified by mixed mode chromatography (e.g., using a mixed mode resin, e.g., a Cato700 resin). In some embodiments, the purification steps comprise removing one or more of serum, host cell DNA, host cell proteins, particles lacking the genetic element, and/or phenol red from the preparation. In some embodiments, the harvested anellovectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### II. Anellovectors

In some aspects, the the disclosure provides compositions and methods of using and making an anellovector, anellovector preparations, and therapeutic compositions. In some embodiments, the anellovector comprises one or more nucleic acids or polypeptides comprising a sequence, structure, and/or function that is based on an Anellovirus (e.g., an Anellovirus as described herein), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety. In some embodiments the anellovector comprises an anellovector, or a component thereof (e.g., a genetic element, e.g., comprising a sequence encoding an effector, and/or a proteinaceous exterior), e.g., as described in PCT Application No. PCT/US19/65995, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as described herein); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:

a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and

b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In embodiments, the promoter element comprises a TATA box. In embodiments, the promoter element is endogenous to a wild-type Anellovirus, e.g., as described herein.

In some embodiments, the genetic element comprises one or more of the following characteristics: single-stranded, circular, negative strand, and/or DNA. In embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In embodiments, introduction of the anellovector, or genetic element comprised therein, decreases level of interferon produced by the cell. In embodiments, introduction of the anellovector, or genetic element comprised therein, into a cell modulates (e.g., increases or decreases) a function of the cell. In embodiments, introduction of the anellovector, or genetic element comprised therein, into a cell modulates (e.g., increases or decreases) the viability of the cell. In embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In embodiments, antibody prevalence is determined according to methods known in the art. In embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

Anelloviruses

In some embodiments, an anellovector, e.g., as described herein, comprises sequences or expression products derived from an Anellovirus. In some embodiments, an anellovector includes one or more sequences or expression products that are exogenous relative to the Anellovirus. In some embodiments, an anellovector includes one or more sequences or expression products that are endogenous relative to the Anellovirus. In some embodiments, an anellovector includes one or more sequences or expression products that are heterologous relative to one or more other sequences or expression products in the anellovector. Anelloviruses generally have single-stranded circular DNA genomes with negative polarity.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of an Anellovirus, e.g., as described herein. In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein. In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (or a splice variant or functional fragment thereof) or a polypeptide encoded by an Anellovirus ORF1 nucleic acid.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table A1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table B1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TAIP nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table C1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TAIP nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table D1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table D1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TAIP nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table E1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table E1.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the anellovector comprises a nucleic acid sequence selected from a sequence as shown in any of Tables A1-M2, or a sequence having at least 70%, 75%, 80%, 85%, 90%, 95%, 96%, 97%, 98%, 99%, or 100% sequence identity thereto. In embodiments, the anellovector comprises a polypeptide comprising a sequence as shown in any of Tables Tables A2-M2, or a sequence having at least 70%, 75%, 80%, 85%, 90%, 95%, 96%, 97%, 98%, 99%, or 100% sequence identity thereto.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any of Tables A-M). In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any of Tables A-M). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any of Tables A2-M2, or an ORF1 or ORF2 amino acid sequence encoded by a nucleic acid sequence as shown in any of Tables A1-M1). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (e.g., an ORF1 amino acid sequence as shown in any of Tables A2-M2, or an ORF1 amino acid sequence encoded by a nucleic acid sequence as shown in any of Tables A1-M1).

In some embodiments, an anellovector as described herein is a chimeric anellovector. In some embodiments, a chimeric anellovector further comprises one or more elements, polypeptides, or nucleic acids from a virus other than an Anellovirus.

In embodiments, the chimeric anellovector comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein). For example, a chimeric anellovector may comprise an ORF1 molecule from one Anellovirus (e.g., a Ring 1 ORF1 molecule, or an ORF1 molecule having at least 75%, 80%, 85%, 90%, 95%, 96%, 97%, 98%, or 99% amino acid sequence identity thereto) and an ORF2 molecule from a different Anellovirus (e.g., a Ring2 ORF2 molecule, or an ORF2 molecule having at least 75%, 80%, 85%, 90%, 95%, 96%, 97%, 98%, or 99% amino acid sequence identity thereto). In another example, a chimeric anellovector may comprise a first ORF1 molecule from one Anellovirus (e.g., a Ring1 ORF1 molecule, or an ORF1 molecule having at least 75%, 80%, 85%, 90%, 95%, 96%, 97%, 98%, or 99% amino acid sequence identity thereto) and a second ORF1 molecule from a different Anellovirus (e.g., a Ring2 ORF1 molecule, or an ORF1 molecule having at least 75%, 80%, 85%, 90%, 95%, 96%, 97%, 98%, or 99% amino acid sequence identity thereto).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In embodiments, the anellovector comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In embodiments, the chimeric ORF1 molecule comprises an ORF1 jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the anellovector comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In embodiments, the anellovector comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In embodiments, the anellovector comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In embodiments, the anellovector comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In embodiments, the anellovector comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In embodiments, the anellovector comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

Additional exemplary Anellovirus genomes, for which sequences or subsequences comprised therein can be utilized in the compositions and methods described herein are described, for example, in PCT Application Nos. PCT/US2018/037379 and PCT/US19/65995 (incorporated herein by reference in their entirety). In some embodiments, the exemplary Anellovirus sequences comprise a nucleic acid sequence as listed in any of Tables A1, A3, A5, A7, A9, A11, B1-B5, 1, 3, 5, 7, 9, 11, 13, 15, or 17 of PCT/US19/65995, incorporated herein by reference. In some embodiments, the exemplary Anellovirus sequences comprise an amino acid sequence as listed in any of Tables A2, A4, A6, A8, A10, A12, C1-C5, 2, 4, 6, 8, 10, 12, 14, 16, or 18 of PCT/US19/65995, incorporated herein by reference. In some embodiments, the exemplary Anellovirus sequences comprise an ORF1 molecule sequence, or a nucleic acid sequence encoding same, e.g., as listed in any of Tables 21, 23, 25, 27, 29, 31, 33, 35, D2, D4, D6, D8, D10, or 37A-37C of PCT/US19/65995, incorporated herein by reference.

TABLE A1
Exemplary Anellovirus nucleic acid sequence
(Alphatorquevirus, Clade 3)

| | |
|---|---|
| Name | Ring 1 |
| Genus/ | Alphatorquevirus, |
| Clade | Clade 3 |
| Accession Number | AJ620231.1 |

Full Sequence: 3753 bp
1 10 20 30 40 50
| | | | | |
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
TTTGCGTGCCAGCAGCTAAGAAAAAACCAACTGCTATGAGCTTCTGGAAA
CCTCCGGTACACAATGTCACGGGGATCCAACGCATGTGGTATGAGTCCTT
TCACCGTGGCCACGCTTCTTTTTGTGGTTGTGGGAATCCTATACTTCACA
TTACTGCACTTGCTGAAACATATGGCCATCCAACAGGCCCGAGACCTTCT
GGGCCACCGGGAGTAGACCCCAACCCCCACATCCGTAGAGCCAGGCCTGC
CCCGGCCGCTCCGGAGCCCTCACAGGTTGATTCGAGACCAGCCCTGACAT
GGCATGGGGATGGTGGAAGCGACGGAGGCGCTGGTGGTTCCGGAAGCGGT
GGACCCGTGGCAGACTTCGCAGACGATGGCCTCGATCAGCTCGTCGCCGC
CCTAGACGACGAAGAGTAAGGAGGCGCAGACGGTGGAGGAGGGGGAGACG
AAAAACAAGGACTTACAGACGCAGGAGACGCTTTAGACGCAGGGGACGAA
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
AAGCAAAACTTATAATAAAACTGTGGCAACCTGCAGTAATTAAAAGATGC
AGAATAAAGGGATACATACCACTGATTATAAGTGGGAACGGTACCTTTGC
CACAAACTTTACCAGTCACATAAATGACAGAATAATGAAAGGCCCCTTCG
GGGGAGGACACAGCACTATGAGGTTCAGCCTCTACATTTTGTTTGAGGAG
CACCTCAGACACATGAACTTCTGGACCAGAAGCAACGATAACCTAGAGCT
AACCAGATACTTGGGGGCTTCAGTAAAAATATACAGGCACCCAGACCAAG
ACTTTATAGTAATATACAACAGAAGAACCCCTCTAGGAGGCAACATCTAC
ACAGCACCCTCTCTACACCCAGGCAATGCCATTTTAGCAAAACACAAAAT
ATTAGTACCAAGTTTACAGACAAGACCAAAGGGTAGAAAAGCAATTAGAC
TAAGAATAGCACCCCCCACACTCTTTACAGACAAGTGGTACTTTCAAAAG
GACATAGCCGACCTCACCCTTTTCAACATCATGGCAGTTGAGGCTGACTT
GCGGTTTCCGTTCTGCTCACCACAAACTGACAACACTTGCATCAGCTTCC
AGGTCCTTAGTTCCGTTTACAACAACTACCTCAGTATTAATACCTTTAAT
AATGACAACTCAGACTCAAAGTTAAAAGAATTTTTAAATAAAGCATTTCC
AACAACAGGCACAAAAGGAACAAGTTTAAATGCACTAAATACATTTAGAA
CAGAAGGATGCATAAGTCACCCACAACTAAAAAAACCAAACCCACAAATA
AACAAACCATTAGAGTCACAATACTTTGCACCTTTAGATGCCCTCTGGGG
AGACCCCATATACTATAATGATCTAAATGAAAACAAAAGTTTGAACGATA
TCATTGAGAAAATACTAATAAAAAACATGATTACATACCATGCAAAACTA
AGAGAATTTCCAAATTCATACCAAGGAAACAAGGCCTTTTGCCACCTAAC
AGGCATATACAGCCCACCATACCTAAACCAAGGCAGAATATCTCCAGAAA
TATTTGGACTGTACACAGAAATAATTTACAACCCTTACACAGACAAAGGA
ACTGGAAACAAAGTATGGATGGACCCACTAACTAAAGAGAACAACATATA
TAAAGAAGGACAGAGCAAATGCCTACTGACTGACATGCCCCTATGGACTT
TACTTTTTGGATATACAGACTGGTGTAAAAAGGACACTAATAACTGGGAC
TTACCACTAAACTACAGACTAGTACTAATATGCCCTTATACCTTTCCAAA
ATTGTACAATGAAAAAGTAAAAGACTATGGGTACATCCCGTACTCCTACA
AATTCGGAGCGGGTCAGATGCCAGACGGCAGCAACTACATACCCTTTCAG
TTTAGAGCAAAGTGGTACCCCACAGTACTACACCAGCAACAGGTAATGGA
GGACATAAGCAGGAGCGGGCCCTTTGCACCTAAGGTAGAAAAACCAAGCA
CTCAGCTGGTAATGAAGTACTGTTTTAACTTTAACTGGGGCGGTAACCCT
ATCATTGAACAGATTGTTAAAGACCCCAGCTTCCAGCCCACCTATGAAAT
ACCCGGTACCGGTAACATCCCTAGAAGAATACAAGTCATCGACCCGCGGG
TCCTGGGACCGCACTACTCGTTCCGGTCATGGGACATGCGCAGACACACA
TTTAGCAGAGCAAGTATTAAGAGAGTGTCAGAACAACAAGAAACTTCTGA
CCTTGTATTCTCAGGCCCAAAAAAGCCTCGGGTCGACATCCCAAAACAAG
AAACCCAAGAAGAAAGCTCACATTCACTCCAAAGAGAATCGAGACCGTGG
GAGACCGAGGAAGAAAGCGAGACAGAAGCCCTCTCGCAAGAGAGCCAAGA
GGTCCCCTTCCAACAGCAGTTGCAGCAGCAGTACCAAGAGCAGCTCAAGC
TCAGACAGGGAATCAAAGTCCTCTTCGAGCAGCTCATAAGGACCCAACAA
GGGGTCCATGTAAACCCATGCCTACGGTAGGTCCCAGGCAGTGGCTGTTT
CCAGAGAGAAAGCCAGCCCCAGCTCCTAGCAGTGGAGACTGGGCCATGGA
GTTTCTCGCAGCAAAAATATTTGATAGGCCAGTTAGAAGCAACCTTAAAG
ATACCCCTTACTACCCATATGTTAAAAACCAATACAATGTCTACTTTGAC
CTTAAATTTGAATAAACAGCAGCTTCAAACTTGCAAGGCCGTGGGAGTTT
CACTGGTCGGTGTCTACCTCTAAAGGTCACTAAGCACTCCGAGCGTAAGC
GAGGAGTGCGACCCTCCCCCCTGGAACAACTTCTTCGGAGTCCGGCGCTA
CGCCTTCGGCTGCGCCGGACACCTCAGACCCCCCCTCCACCCGAAACGCT
TGCGCGTTTCGGACCTTCGGCGTCGGGGGGGTCGGGAGCTTTATTAAACG
GACTCCGAAGTGCTCTTGGACACTGAGGGGGTGAACAGCAACGAAAGTGA
GTGGGGCCAGACTTCGCCATAAGGCCTTTATCTTCTTGCCATTTGTCAGT
GTCCGGGGTCGCCATAGGCTTCGGGCTCGTTTTTAGGCCTTCCGGACTAC
AAAAATCGCCATTTTGGTGACGTCACGGCCGCCATCTTAAGTAGTTGAGG
CCG (SEQ ID NO: 16)

### Annotations:

Putative Domain Base range

### TATA Box 83-88

### Cap Site 104-111

### Transcriptional Start Site 111

### 5′ UTR Conserved Domain 170-240

### ORF2 336-719

### ORF2/2 336-715; 2363-2789

### ORF2/3 336-715; 2565-3015

### ORF2t/3 336-388; 2565-3015

### ORF1 599-2830

### ORF1/1 599-715; 2363-2830

### ORF1/2 599-715; 2565-2789

TABLE A2
Exemplary Anellovirus amino acid sequences
(Alphatorquevirus, Clade 3)
Ring1 (Alphatorquevirus Clade 3)

| | |
|---|---|
| ORF2 | MSFWKPPVHNVTGIQRMWYESFHRG |
| | HASFCGCGNPILHITALAETYGHPT |
| | GPRPSGPPGVDPNPHIRRARPAPAA |
| | PEPSQVDSRPALTWHGDGGSDGGAG |
| | GSGSGGPVADFADDGLDQLVAALDD |
| | EE |
| | (SEQ ID NO: 17) |
| ORF2/2 | MSFWKPPVHNVTGIQRMWYESFHRG |
| | HASFCGCGNPILHITALAETYGHPT |
| | GPRPSGPPGVDPNPHIRRARPAPAA |
| | PEPSQVDSRPALTWHGDGGSDGGAG |
| | GSGSGGPVADFADDGLDQLVAALDD |
| | EELLKTPASSPPMKYPVPVTSLEEY |
| | KSSTRGSWDRTTRSGHGTCADTHLA |
| | EQVLRECQNNKKLLTLYSQAQKSLG |
| | STSQNKKPKKKAHIHSKENRDRGRP |
| | RKKARQKPSRKRAKRSPSNSSCSSS |
| | TKSSSSSDRESKSSSSSS |
| | (SEQ ID NO: 18) |
| ORF2/3 | MSFWKPPVHNVTGIQRMWYESFHRG |
| | HASFCGCGNPILHITALAETYGHPT |
| | GPRPSGPPGVDPNPHIRRARPAPAA |
| | PEPSQVDSRPALTWHGDGGSDGGAG |
| | GSGSGGPVADFADDGLDQLVAALDD |
| | EEPKKASGRHPKTRNPRRKLTFTPK |
| | RIETVGDRGRKRDRSPLAREPRGPL |
| | PTAVAAAVPRAAQAQTGNQSPLRAA |
| | HKDPTRGPCKPMPTVGPRQWLFPER |
| | KPAPAPSSGDWAMEFLAAKIFDRPV |
| | RSNLKDTPYYPYVKNQYNVYFDLKF |
| | E |
| | (SEQ ID NO: 19) |
| ORF2t/3 | MSFWKPPVHNVTGIQRMWPKKASGR |
| | HPKTRNPRRKLTFTPKRIETVGDRG |
| | RKRDRSPLAREPRGPLPTAVAAAVP |
| | RAAQAQTGNQSPLRAAHKDPTRGPC |
| | KPMPTVGPRQWLFPERKPAPAPSSG |
| | DWAMEFLAAKIFDRPVRSNLKDTPY |
| | YPYVKNQYNVYFDLKFE |
| | (SEQ ID NO: 20) |
| ORF1 | MAWGWWKRRRRWWFRKRWTRGRLRR |
| | RWPRSARRRPRRRRVRRRRRWRRGR |
| | RKTRTYRRRRRFRRRGRKAKLIIKL |
| | WQPAVIKRCRIKGYIPLIISGNGTF |
| | ATNFTSHINDRIMKGPFGGGHSTMR |
| | FSLYILFEEHLRHMNFWTRSNDNLE |
| | LTRYLGASVKIYRHPDQDFIVIYNR |
| | RTPLGGNIYTAPSLHPGNAILAKHK |
| | ILVPSLQTRPKGRKAIRLRIAPPTL |
| | FTDKWYFQKDIADLTLFNIMAVEAD |
| | LRFPFCSPQTDNTCISFQVLSSVYN |
| | NYLSINTFNNDNSDSKLKEFLNKAF |
| | PTTGTKGTSLNALNTFRTEGCISHP |
| | QLKKPNPQINKPLESQYFAPLDALW |
| | GDPIYYNDLNENKSLNDIIEKILIK |
| | NMITYHAKLREFPNSYQGNKAFCHL |
| | TGIYSPPYLNQGRISPEIFGLYTEI |
| | IYNPYTDKGTGNKVWMDPLTKENNI |
| | YKEGQSKCLLTDMPLWTLLFGYTDW |
| | CKKDTNNWDLPLNYRLVLICPYTFP |
| | KLYNEKVKDYGYIPYSYKFGAGQMP |
| | DGSNYIPFQFRAKWYPTVLHQQQVM |
| | EDISRSGPFAPKVEKPSTQLVMKYC |
| | FNFNWGGNPIIEQIVKDPSFQPTYE |
| | IPGTGNIPRRIQVIDPRVLGPHYSF |
| | RSWDMRRHTFSRASIKRVSEQQETS |
| | DLVFSGPKKPRVDIPKQETQEESSH |
| | SLQRESRPWETEEESETEALSQESQ |
| | EVPFQQQLQQQYQEQLKLRQGIKVL |
| | FEQLIRTQQGVHVNPCLR |
| | (SEQ ID NO: 21) |
| ORF 1/1 | MAWGWWKRRRRWWFRKRWTRGRLRR |
| | RWPRSARRRPRRRRIVKDPSFQPTY |
| | EIPGTGNIPRRIQVIDPRVLGPHYS |
| | FRSWDMRRHTFSRASIKRVSEQQET |
| | SDLVFSGPKKPRVDIPKQETQEESS |
| | HSLQRESRPWETEEESETEALSQES |
| | QEVPFQQQLQQQYQEQLKLRQGIKV |
| | LFEQLIRTQQGVHVNPCLR |
| | (SEQ ID NO: 22) |
| ORF 1/2 | MAWGWWKRRRRWWFRKRWTRGRLRR |
| | RWPRSARRRPRRRRAQKSLGSTSQN |
| | KKPKKKAHIHSKENRDRGRPRKKAR |
| | QKPSRKRAKRSPSNSSCSSSTKSSS |
| | SSDRESKSSSSSS |
| | (SEQ ID NO: 23) |

TABLE B1
Exemplary Anellovirus nucleic
acid sequence (Betatorquevirus)

| | |
|---|---|
| Name | Ring2 |
| Genus/Clade | Betatorquevirus |
| Accession Number | JX134045.1 |

Full Sequence: 2797 bp
1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)

### Annotations:

Putative Domain Base range

### TATA Box 237-243

### Cap Site 260-267

### Transcriptional Start Site 267

### 5′ UTR Conserved Domain 323-393

### ORF2 424-723

### ORF2/2 424-719; 2274-2589

### ORF2/3 424-719; 2449-2812

### ORF1 612-2612

### ORF1/1 612-719; 2274-2612

### ORF1/2 612-719; 2449-2589

Three open-reading frame region 2441-2586

### Poly(A) Signal 2808-2813

GC-rich region 2868-2929

TABLE B2
Exemplary Anellovirus amino acid
sequences (Betatorquevirus)
Ring2 (Betatorquevirus)

| | |
|---|---|
| ORF2 | MSDCFKPTCYNNKTKQTHWINNLHL |
| | THDLICFCPTPTRHLLLALAEQQET |
| | IEVSKQEKEKITRCLITTEEDGTTT |
| | DVLDGMDEVGLDALFAEDFEEKEG |
| | (SEQ ID NO: 55) |
| ORF2/2 | MSDCFKPTCYNNKTKQTHWINNLHL |
| | THDLICFCPTPTRHLLLALAEQQET |
| | IEVSKQEKEKITRCLITTEEDGTTT |
| | DVLDGMDEVGLDALFAEDFEEKEGF |
| | NIPYPVTSMKQLRYRVQGKPQNPSY |
| | TPSTIDTGTTQQQLCHELAKTGHLK |
| | TLFLKLQSQIDSNCSNKPSNACKSR |
| | KKRRRKKKKKYSSSSATSDSSSSCT |
| | ESE |
| | (SEQ ID NO: 56) |
| ORF2/3 | MSDCFKPTCYNNKTKQTHWINNLHL |
| | THDLICFCPTPTRHLLLALAEQQET |
| | IEVSKQEKEKITRCLITTEEDGTTT |
| | DVLDGMDEVGLDALFAEDFEEKEGA |
| | RSTATAQTSPRMPANLGRNAGEKRK |
| | RSTAAHQQPQTAAAAVQRANNIIIK |
| | GPITFNCVKKVKLFDDKPKNRRFTP |
| | EEFETELQIAKWLKRPPRSFVNDPP |
| | FYPWLPPEPVVNFKLNFTE |
| | (SEQ ID NO: 57) |
| ORF1 | MPYYYRRRRYNYRRPRWYGRGWIRR |
| | PFRRRFRRKRRVRPTYTTIPLKQWQ |
| | PPYKRTCYIKGQDCLIYYSNLRLGM |
| | NSTMYEKSIVPVHWPGGGSFSVSML |
| | TLDALYDIHKLCRNWWTSTNQDLPL |
| | VRYKGCKITFYQSTFTDYIVRIHTE |
| | LPANSNKLTYPNTHPLMMMMSKYKH |
| | IIPSRQTRRKKKPYTKIFVKPPPQF |
| | ENKWYFATDLYKIPLLQIHCTACNL |
| | QNPFVKPDKLSNNVTLWSLNTISIQ |
| | NRNMSVDQGQSWPFKILGTQSFYFY |
| | FYTGANLPGDTTQIPVADLLPLTNP |
| | RINRPGQSLNEAKITDHITFTEYKN |
| | KFTNYWGNPFNKHIQEHLDMILYSL |
| | KSPEAIKNEWTTENMKWNQLNNAGT |
| | MALTPFNEPIFTQIQYNPDRDTGED |
| | TQLYLLSNATGTGWDPPGIPELILE |
| | GFPLWLIYWGFADFQKNLKKVTNID |
| | TNYMLVAKTKFTQKPGTFYLVILND |
| | TFVEGNSPYEKQPLPEDNIKWYPQV |
| | QYQLEAQNKLLQTGPFTPNIQGQLS |
| | DNISMFYKFYFKWGGSPPKAINVEN |
| | PAHQIQYPIPRNEHETTSLQSPGEA |
| | PESILYSFDYRHGNYTTTALSRISQ |
| | DWALKDTVSKITEPDRQQLLKQALE |
| | CLQISEETQEKKEKEVQQLISNLRQ |
| | QQQLYRERIISLLKDQ |
| | (SEQ ID NO: 58) |
| ORF 1/1 | MPYYYRRRRYNYRRPRWYGRGWIRR |
| | PFRRRFRRKRRIQYPIPRNEHETTS |
| | LQSPGEAPESILYSFDYRHGNYTTT |
| | ALSRISQDWALKDTVSKITEPDRQQ |
| | LLKQALECLQISEETQEKKEKEVQQ |
| | LISNLRQQQQLYRERIISLLKDQ |
| | (SEQ ID NO: 59) |
| ORF 1/2 | MPYYYRRRRYNYRRPRWYGRGWIRR |
| | PFRRRFRRKRRSQIDSNCSNKPSNA |
| | CKSRKKRRRKKKKKYSSSSATSDSS |
| | SSCTESE |
| | (SEQ ID NO: 60) |

TABLE C1
Exemplary Anellovirus nucleic acid sequence
(Gammatorquevirus)

| | |
|---|---|
| Name | Ring4 |
| Genus/Clade | Gammatorquevirus |
| Accession Number | |

Full Sequence: 3176 bp
1 10 20 30 40 50
| | | | | |
TAAAATGGCGGGAGCCAATCATTTTATACTTTCACTTTCCAATTAAAAAT
GGCCACGTCACAAACAAGGGGTGGAGCCATTTAAACTATATAACTAAGTG
GGGTGGCGAATGGCTGAGTTTACCCCGCTAGACGGTGCAGGGACCGGATC
GAGCGCAGCGAGGAGGTCCCCGGCTGCCCATGGGCGGGAGCCGAGGTGAG
TGAAACCACCGAGGTCTAGGGGCAATTCGGGCTAGGGCAGTCTAGCGGAA
CGGGCAAGAAACTTAAAACAATATTTGTTTTACAGATGGTTAGTATATCC
TCAAGTGATTTTTTTAAGAAAACGAAATTTAATGAGGAGACGCAGAACCA
AGTATGGATGTCTCAAATTGCTGACTCTCATGATAATATCTGCAGTTGCT
GGCATCCATTTGCTCACCTTCTTGCTTCCATATTTCCTCCTGGCCACAAA
GATCGTGATCTTACTATTAACCAAATTCTTCTAAGAGATTATAAAGAAAA
ATGCCATTCTGGTGGAGAAGAAGGAGAAAATTCTGGACCAACAACAGGTT
TAATTACACCAAAAGAAGAAGATATAGAAAAAGATGGCCCAGAAGGCGCC
GCAGAAGAAGACCATACAGACGCCCTGTTCGCCGCCGCCGTAGAAAACTT
CGAAAGGTAAAGAGAAAAAAAAAATCTTTAATTGTTAGACAATGGCAACC
AGACAGTATAAGAACTTGTAAAATTATAGGACAGTCAGCTATAGTTGTTG
GGGCTGAAGGAAAGCAAATGTACTGTTATACTGTCAATAAGTTAATTAAT
GTGCCCCCAAAAACACCATATGGGGGAGGCTTTGGAGTAGACCAATACAC
ACTGAAATACTTATATGAAGAATACAGATTTGCACAAAACATTTGGACAC
AATCTAATGTACTGAAAGACTTATGCAGATACATAAATGTTAAGCTAATA
TTCTACAGAGACAACAAAACAGACTTTGTCCTTTCCTATGACAGAAACCC
ACCTTTTCAACTAACAAAATTTACATACCCAGGAGCACACCCACAACAAA
TCATGCTTCAAAAACACCACAAATTCATACTATCACAAATGACAAAGCCT
AATGGAAGACTAACAAAAAAACTCAAAATTAAACCTCCTAAACAAATGCT
TTCTAAATGGTTCTTTTCAAAACAATTCTGTAAATACCCTTTACTATCTC
TTAAAGCTTCTGCACTAGACCTTAGGCACTCTTACCTAGGCTGCTGTAAT
GAAAATCCACAGGTATTTTTTTATTATTTAAACCATGGATACTACACAAT
AACAAACTGGGGAGCACAATCCTCAACAGCATACAGACCTAACTCCAAGG
TGACAGACACAACATACTACAGATACAAAAATGACAGAAAAAATATTAAC
ATTAAAAGCCATGAATACGAAAAAAGTATATCATATGAAAACGGTTATTT
TCAATCTAGTTTCTTACAAACACAGTGCATATATACCAGTGAGCGTGGTG
AAGCCTGTATAGCAGAAAAACCACTAGGAATAGCTATTTACAATCCAGTA
AAAGACAATGGAGATGGTAATATGATATACCTTGTAAGCACTCTAGCAAA
CACTTGGGACCAGCCTCCAAAAGACAGTGCTATTTTAATACAAGGAGTAC
CCATATGGCTAGGCTTATTTGGATATTTAGACTACTGTAGACAAATTAAA
GCTGACAAAACATGGCTAGACAGTCATGTACTAGTAATTCAAAGTCCTGC
TATTTTTACTTACCCAAATCCAGGAGCAGGCAAATGGTATTGTCCACTAT
CACAAAGTTTTATAAATGGCAATGGTCCGTTTAATCAACCACCTACACTG
CTACAAAAAGCAAAGTGGTTTCCACAAATACAATACCAACAAGAAATTAT
TAATAGCTTTGTAGAATCAGGACCATTTGTTCCCAAATATGCAAATCAAA
CTGAAAGCAACTGGGAACTAAAATATAAATATGTTTTTACATTTAAGTGG
GGTGGACCACAATTCCATGAACCAGAAATTGCTGACCCTAGCAAACAAGA
GCAGTATGATGTCCCCGATACTTTCTACCAAACAATACAAATTGAAGATC
CAGAAGGACAAGACCCCAGATCTCTCATCCATGATTGGGACTACAGACGA
GGCTTTATTAAAGAAAGATCTCTTAAAAGAATGTCAACTTACTTCTCAAC
TCATACAGATCAGCAAGCAACTTCAGAGGAAGACATTCCCAAAAAGAAAA
AGAGAATTGGACCCCAACTCACAGTCCCACAACAAAAAGAAGAGGAGACA
CTGTCATGTCTCCTCTCTCTCTGCAAAAAAGATACCTTCCAAGAAACAGA
GACACAAGAAGACCTCCAGCAGCTCATCAAGCAGCAGCAGGAGCAGCAGC
TCCTCCTCAAGAGAAACATCCTCCAGCTCATCCACAAACTAAAAGAGAAT
CAACAAATGCTTCAGCTTCACACAGGCATGTTACCTTAACCAGATTTAAA
CCTGGATTTGAAGAGCAAACAGAGAGAGAATTAGCAATTATATTTCATAG
GCCCCCTAGAACCTACAAAGAGGACCTTCCATTCTATCCCTGGCTACCAC
CTGCACCCCTTGTACAATTTAACCTTAACTTCAAAGGCTAGGCCAACAAT
GTACACTTAGTAAAGCATGTTTATTAAAGCACAACCCCCAAAATAAATGT
AAAAATAAAAAAAAAAAAAAAAAAATAAAAAATTGCAAAAATTCGGCGCT
CGCGCGCATGTGCGCCTCTGGCGCAAATCACGCAACGCTCGCGCGCCCGC
GTATGTCTCTTTACCACGCACCTAGATTGGGGTGCGCGCGCTAGCGCGCG
CACCCCAATGCGCCCCGCCCTCGTTCCGACCCGCTTGCGCGGGTCGGACC
ACTTCGGGCTCGGGGGGGCGCGCCTGCGGCGCTTTTTTACTAAACAGACT
CCGAGCCGCCATTTGGCCCCCTAAGCTCCGCCCCCCTCATGAATATTCAT
AAAGGAAACCACATAATTAGAATTGCCGACCACAAACTGCCATATGCTAA
TTAGTTCCCCTTTTACAAAGTAAAAGGGGAAGTGAACATAGCCCCACACC
CGCAGGGGCAAGGCCCCGCACCCCTACGTCACTAACCACGCCCCCGCCGC
CATCTTGGGTGCGGCAGGGCGGGGGC (SEQ ID NO: 886)

### Annotations:

Putative Domain Base range

### TATA Box 87-93

### Cap Site 110-117

### Transcriptional Start Site 117

### 5′ UTR Conserved Domain 185-254

### ORF2 286-660

### ORF2/2 286-656; 1998-2442

### ORF2/3 286-656; 2209-2641

### TAIP 385-484

### ORF1 501-2489

### ORF1/1 501-656; 1998-2489

### ORF1/2 501-656; 2209-2442

Three open-reading frame region 2209-2439

### Poly(A) Signal 2672-2678

GC-rich region 3076-3176

Exemplary Anellovirus amino acid
sequences (Gammatorquevirus)
Ring4 (Gammatorquevirus)

| | |
|---|---|
| ORF2 | MVSISSSDFFKKTKFNEETQNQVWM |
| | SQIADSHDNICSCWHPFAHLLASIF |
| | PPGHKDRDLTINQILLRDYKEKCHS |
| | GGEEGENSGPTTGLITPKEEDIEKD |
| | GPEGAAEEDHTDALFAAAVENFER |
| | (SEQ ID NO: 887) |
| ORF2/2 | MVSISSSDFFKKTKFNEETQNQVWM |
| | SQIADSHDNICSCWHPFAHLLASIF |
| | PPGHKDRDLTINQILLRDYKEKCHS |
| | GGEEGENSGPTTGLITPKEEDIEKD |
| | GPEGAAEEDHTDALFAAAVENFESG |
| | VDHNSMNQKLLTLANKSSMMSPILS |
| | TKQYKLKIQKDKTPDLSSMIGTTDE |
| | ALLKKDLLKECQLTSQLIQISKQLQ |
| | RKTFPKRKRELDPNSQSHNKKKRRH |
| | CHVSSLSAKKIPSKKQRHKKTSSSS |
| | SSSSRSSSSSSRETSSSSSTN |
| | (SEQ ID NO: 888) |
| ORF2/3 | MVSISSSDFFKKTKFNEETQNQVWM |
| | SQIADSHDNICSCWHPFAHLLASIF |
| | PPGHKDRDLTINQILLRDYKEKCHS |
| | GGEEGENSGPTTGLITPKEEDIEKD |
| | GPEGAAEEDHTDALFAAAVENFERS |
| | ASNFRGRHSQKEKENWTPTHSPTTK |
| | RRGDTVMSPLSLQKRYLPRNRDTRR |
| | PPAAHQAAAGAAAPPQEKHPPAHPQ |
| | TKRESTNASASHRHVTLTRFKPGFE |
| | EQTERELAIIFHRPPRTYKEDLPFY |
| | PWLPPAPLVQFNLNFKG |
| | (SEQ ID NO: 889) |
| TAIP | MRRRRTKYGCLKLLTLMIISAVAGI |
| | HLLTFLLPYFLLATKIVILLLTKFF |
| | (SEQ ID NO: 890) |
| ORF1 | MPFWWRRRRKFWTNNRFNYTKRRRY |
| | RKRWPRRRRRRRPYRRPVRRRRRKL |
| | RKVKRKKKSLIVRQWQPDSIRTCKI |
| | IGQSAIVVGAEGKQMYCYTVNKLIN |
| | VPPKTPYGGGFGVDQYTLKYLYEEY |
| | RFAQNIWTQSNVLKDLCRYINVKLI |
| | FYRDNKTDFVLSYDRNPPFQLTKFT |
| | YPGAHPQQIMLQKHHKFILSQMTKP |
| | NGRLTKKLKIKPPKQMLSKWFFSKQ |
| | FCKYPLLSLKASALDLRHSYLGCCN |
| | ENPQVFFYYLNHGYYTITNWGAQSS |
| | TAYRPNSKVTDTTYYRYKNDRKNIN |
| | IKSHEYEKSISYENGYFQSSFLQTQ |
| | CIYTSERGEACIAEKPLGIAIYNPV |
| | KDNGDGNMIYLVSTLANTWDQPPKD |
| | SAILIQGVPIWLGLFGYLDYCRQIK |
| | ADKTWLDSHVLVIQSPAIFTYPNPG |
| | AGKWYCPLSQSFINGNGPFNQPPTL |
| | LQKAKWFPQIQYQQEIINSFVESGP |
| | FVPKYANQTESNWELKYKYVFTFKW |
| | GGPQFHEPEIADPSKQEQYDVPDTF |
| | YQTIQIEDPEGQDPRSLIHDWDYRR |
| | GFIKERSLKRMSTYFSTHTDQQATS |
| | EEDIPKKKKRIGPQLTVPQQKEEET |
| | LSCLLSLCKKDTFQETETQEDLQQL |
| | IKQQQEQQLLLKRNILQLIHKLKEN |
| | QQMLQLHTGMLP |
| | (SEQ ID NO: 891) |
| ORF 1/1 | MPFWWRRRRKFWTNNRFNYTKRRRY |
| | RKRWPRRRRRRRPYRRPVRRRRRKL |
| | RKWGGPQFHEPEIADPSKQEQYDVP |
| | DTFYQTIQIEDPEGQDPRSLIHDWD |
| | YRRGFIKERSLKRMSTYFSTHTDQQ |
| | ATSEEDIPKKKKRIGPQLTVPQQKE |
| | EETLSCLLSLCKKDTFQETETQEDL |
| | QQLIKQQQEQQLLLKRNILQLIHKL |
| | KENQQMLQLHTGMLP |
| | (SEQ ID NO: 892) |
| ORF 1/2 | MPFWWRRRRKFWTNNRFNYTKRRRY |
| | RKRWPRRRRRRRPYRRPVRRRRRKL |
| | RKISKQLQRKTFPKRKRELDPNSQS |
| | HNKKKRRHCHVSSLSAKKIPSKKQR |
| | HKKTSSSSSSSSRSSSSSSRETSSS |
| | SSTN |
| | (SEQ ID NO: 893) |

TABLE D1
Exemplary Anellovirus nucleic acid
sequence (Betatorquevirus)

| | |
|---|---|
| Name | Ring9 |
| Genus/Clade | Betatorquevirus |
| Accession Number | MH649263.1 |

Full Sequence: 2845 bp
1 10 20 30 40 50
| | | | | |
TTATTAATATTCAACAGGAAAACCACCTAATTTAAATTGCCGACCACAAA
CCGTCACTAACTTCCTTATTTAACATTACTTCCCTTTTAACCAATGAATA
TTCATACAACACATCACACTTCCTGGGAGGAGACATAAAACTATATAACT
AACTACACAGACGAATGGCTGAGTTTATGCCGCTAGACGGAGGACGCACA
GCTACTGCTGCGACCTGAACTTGGGCGGGTGCCGAAGGTGAGTGTAACCA
CCGTAGTCAAGGGGCAATTCGGGCTAGTTCAGTCTAGCGGAACGGGCAAG
ATTATTAATACAAACTTATTTTTACAGATGAGCAAACAACTAAAACCAAC
TTTATACAAAGACAAATCATTGGAATTACAATGGCTAAACAACATTTTTA
GCTCTCACGACCTGTGCTGCGGCTGCAACGATCCAGTTTTACATTTACTG
ATTTTAATTAACAAAACCGGAGAAGCACCTAAACCAGAAGAAGACATTAA
AAATATAAAATGCCTCCTTACTGGCGCCAAAAATACTACCGAAGAAGATA
TAGACCTTTCTCCTGGAGAACTAGAAGAATTATTCAAAGAAGAAAAAGAT
GGAGATACCGCAAACCAAGAAAAACATACTGGAGAAGAAAACTGCGGGTA
AGAAAACGTTTTTATAAAAGAAAGTTAAAAAAAATTGTACTTAAACAGTT
TCAACCAAAAATTATTAGAAGATGTACAATATTTGGAACAATCTGCCTAT
TTCAAGGCTCTCCAGAAAGAGCCAACAATAATTATATTCAAACAATCTAC
TCCTACGTACCAGATAAAGAACCAGGAGGAGGGGGATGGACTTTAATAAC
TGAAAGCTTAAGTAGTTTATGGGAAGACTGGGAACATTTAAAAAATGTAT
GGACTCAAAGTAACGCTGGTTTACCACTTGTAAGATACGGGGGAGTAACA
TTATACTTTTATCAATCTGCCTATACTGACTATATTGCTCAAGTTTTCAA
CTGTTATCCTATGACAGACACAAAATACACACATGCAGACTCAGCACCAA
ACAGAATGTTATTAAAAAAACATGTAATAAGAGTACCTAGCAGAGAAACA
CGCAAAAAAAGAAAGCCATACAAAAGAGTTAGAGTAGGACCTCCTTCTCA
AATGCAAAACAAATGGTACTTTCAAAGAGACATATGTGAAATACCATTAA
TAATGATTGCAGCCACAGCCGTTGACTTTAGATATCCCTTTTGTGCAAGC
GACTGTGCTAGTAACAACTTAACTCTAACATGTTTAAACCCACTATTGTT
TCAAAACCAAGACTTTGACCACCCATCCGATACACAAGGCTACTTTCCAA
AACCTGGAGTATATCTATACTCAACACAAAGAAGTAACAAGCCAAGTTCT
TCAGACTGTATATACTTAGGAAACACAAAAGACAATCAAGAAGGTAAATC
TGCAAGTAGTCTAATGACTCTAAAAACACAAAAAATAACAGATTGGGGAA
ATCCATTTTGGCATTATTATATAGACGGTTCTAAAAAAATATTTTCTTAC
TTTAAACCCCCATCACAATTAGACAGCAGCGACTTTGAACACATGACAGA
ATTAGCAGAACCAATGTTTATACAAGTTAGATACAACCCAGAAAGAGACA
CAGGACAAGGAAACTTAATATACGTAACAGAAAACTTTAGAGGACAACAC
TGGGACCCTCCATCTAGTGACAACCTAAAATTAGATGGATTTCCCTTATA
TGACATGTGCTGGGGTTTCATAGACTGGATAGAAAAAGTTCATGAAACAG
AAAACTTACTTACCAACTACTGCTTCTGTATTAGAAGCAGCGCTTTCAAT
GAAAAAAAAACAGTTTTTATACCTGTAGATCATTCATTTTTAACAGGTTT
TAGCCCATATGAAACTCCAGTTAAATCATCAGACCAAGCTCACTGGCACC
CACAAATAAGATTTCAAACAAAATCAATAAATGACATTTGTTTAACAGGC
CCCGGTTGTGCTAGGTCCCCATATGGCAATTACATGCAGGCAAAAATGAG
TTATAAATTTCATGTAAAATGGGGAGGATGTCCAAAAACTTATGAAAAAC
CATATGATCCTTGTTCACAGCCCAATTGGACTATTCCCCATAACCTCAAT
GAAACAATACAAATCCAGAATCCAAACACATGCCCACAAACAGAACTCCA
AGAATGGGACTGGCGACGTGATATTGTTACAAAAAAAGCTATCGAAAGAA
TTAGACAACACACGGAACCTCATGAAACTTTGCAAATCTCTACAGGTTCC
AAACACAACCCACCAGTACACAGACAAACATCACCGTGGACGGACTCAGA
AACGGACTCGGAAGAGGAAAAAGACCAAACACAAGAGATCCAGATCCAGC
TCAACAAGCTCAGAAAGCATCAACAGCATCTCAAGCAGCAGCTCAAGCAG
TACCTGAAACCCCAAAATATAGAATAGTTGCAAGCAACATAAAAGTTGAA
CTTTTTCCTACTAAAAAACCTTTTAAAAACAGACGCTTTACTCCTTCTGA
AAGAGAAACAGAAAGACAATGTGCTAAAGCTTTTTGTAGACCAGAAAGAC
ATTTCTTTTATGATCCTCCTTTTTACCCTTACTGTGTACCTGAACCTATT
GTAAACTTTGCTTTGGGATATAAAATTTAAGGCCAACAAATTTCACTTAG
TGGTGTCTGTTTATTAAAGTTTAACCTTAATAAGCATACTCCGCCTCCCT
ACATTAAGGCGCCAAAAGGGGGCTCCGCCCCCTTAAACCCCAAGGGGGCT
CCGCCCCCTTAAACCCCCAAGGGGGCTCCGCCCCCTTACACCCCC
(SEQ ID NO: 1001)

### Annotations:

Putative Domain Base range

### TATA Box 142-148

### Initiation Element 162-177

### Transcriptional Start Site 172

### 5′ UTR Conserved Domain 226-296

### ORF2 328-651

### ORF2/2 328-647; 2121-2457

### ORF2/3 328-647; 2296-2680

### ORF1 510-2477

### ORF1/1 510-647; 2121-2477

### ORF1/2 510-647; 2296-2457

Three open-reading frame region 2296-2454
GC-rich region 2734-2845

TABLE D2
Exemplary Anellovirus amino acid
sequences (Betatorquevirus)
Ring9 (Betatorquevirus)

| | |
|---|---|
| ORF2 | MSKQLKPTLYKDKSLELQWLNNIFS |
| | SHDLCCGCNDPVLHLLILINKTGEA |
| | PKPEEDIKNIKCLLTGAKNTTEEDI |
| | DLSPGELEELFKEEKDGDTANQEKH |
| | TGEENCG |
| | (SEQ ID NO: 1002) |
| ORF2/2 | MSKQLKPTLYKDKSLELQWLNNIFS |
| | SHDLCCGCNDPVLHLLILINKTGEA |
| | PKPEEDIKNIKCLLTGAKNTTEEDI |
| | DLSPGELEELFKEEKDGDTANQEKH |
| | TGEENCGPIGLFPITSMKQYKSRIQ |
| | THAHKQNSKNGTGDVILLQKKLSKE |
| | LDNTRNLMKLCKSLQVPNTTHQYTD |
| | KHHRGRTQKRTRKRKKTKHKRSRSS |
| | STSSESINSISSSSSSST |
| | (SEQ ID NO: 1003) |
| ORF2/3 | MSKQLKPTLYKDKSLELQWLNNIFS |
| | SHDLCCGCNDPVLHLLILINKTGEA |
| | PKPEEDIKNIKCLLTGAKNTTEEDI |
| | DLSPGELEELFKEEKDGDTANQEKH |
| | TGEENCGFQTQPTSTQTNITVDGLR |
| | NGLGRGKRPNTRDPDPAQQAQKAST |
| | ASQAAAQAVPETPKYRIVASNIKVE |
| | LFPTKKPFKNRRFTPSERETERQCA |
| | KAFCRPERHFFYDPPFYPYCVPEPI |
| | VNFALGYKI |
| | (SEQ ID NO: 1004) |
| ORF1 | MPPYWRQKYYRRRYRPFSWRTRRII |
| | QRRKRWRYRKPRKTYWRRKLRVRKR |
| | FYKRKLKKIVLKQFQPKIIRRCTIF |
| | GTICLFQGSPERANNNYIQTIYSYV |
| | PDKEPGGGGWTLITESLSSLWEDWE |
| | HLKNVWTQSNAGLPLVRYGGVTLYF |
| | YQSAYTDYIAQVFNCYPMTDTKYTH |
| | ADSAPNRMLLKKHVIRVPSRETRKK |
| | RKPYKRVRVGPPSQMQNKWYFQRDI |
| | CEIPLIMIAATAVDFRYPFCASDCA |
| | SNNLTLTCLNPLLFQNQDFDHPSDT |
| | QGYFPKPGVYLYSTQRSNKPSSSDC |
| | IYLGNTKDNQEGKSASSLMTLKTQK |
| | ITDWGNPFWHYYIDGSKKIFSYFKP |
| | PSQLDSSDFEHMTELAEPMFIQVRY |
| | NPERDTGQGNLIYVTENFRGQHWDP |
| | PSSDNLKLDGFPLYDMCWGFIDWIE |
| | KVHETENLLTNYCFCIRSSAFNEKK |
| | TVFIPVDHSFLTGFSPYETPVKSSD |
| | QAHWHPQIRFQTKSINDICLTGPGC |
| | ARSPYGNYMQAKMSYKFHVKWGGCP |
| | KTYEKPYDPCSQPNWTIPHNLNETI |
| | QIQNPNTCPQTELQEWDWRRDIVTK |
| | KAIERIRQHTEPHETLQISTGSKHN |
| | PPVHRQTSPWTDSETDSEEEKDQTQ |
| | EIQIQLNKLRKHQQHLKQQLKQYLK |
| | PQNIE |
| | (SEQ ID NO: 1005) |
| ORF 1/1 | MPPYWRQKYYRRRYRPFSWRTRRII |
| | QRRKRWRYRKPRKTYWRRKLRPNWT |
| | IPHNLNETIQIQNPNTCPQTELQEW |
| | DWRRDIVTKKAIERIRQHTEPHETL |
| | QISTGSKHNPPVHRQTSPWTDSETD |
| | SEEEKDQTQEIQIQLNKLRKHQQHL |
| | KQQLKQYLKPQNIE |
| | (SEQ ID NO: 1006) |
| ORF 1/2 | MPPYWRQKYYRRRYRPFSWRTRRII |
| | QRRKRWRYRKPRKTYWRRKLRVPNT |
| | THQYTDKHHRGRTQKRTRKRKKTKH |
| | KRSRSSSTSSESINSISSSSSSST |
| | (SEQ ID NO: 1007) |

TABLE E1
Exemplary Anellovirus nucleic acid sequence (Betatorquevirus)

| | |
|---|---|
| Name | Ring 10 |
| Genus/Clade | Betatorquevirus |
| Accession Number | JX134044.1 |

Full Sequence: 2912 bp
1 10 20 30 40 50
| | | | | |
TAATAAATATTCAACAGGAAAACCACCTAATTTAAATTGCCGACCACAAA
CCGTCACTTAGTTCCTCTTTTTCCACAACTTCCTCTTTTACTAATGAATA
TTCATGTAATTAATTAATAATCACCGTAATTCCGGGGAGGAGCCTTTAAA
CTATAAAACTAACTACACATTCGAATGGCTGAGTTTATGCCGCCAGACGG
AGACGGGATCACTTCAGTGACTCCAGGCTGATCAAGGGCGGGTGCCGAAG
GTGAGTGAAACCACCGTAGTCAAGGGGCAATTCGGGCTAGATCAGTCTGG
CGGAACGGGCAAGAAACTTAAAATGTACTTTATTTTACAGAAATGTTCAA
ATCTCCAACATACTTAACAACTAAAGGCAAAAACAATGCCTTAATCAACT
GCTTCGTTGGAGACCACGATCTTCTGTGCAGCTGTAACAATCCTGCCTAC
CATTGCCTCCAAATACTTGCAACTACCTTAGCACCTCAACTAAAACAAGA
AGAAAAACAACAAATAATACAATGCCTTGGTGGTACAGACGCCGTAGCTA
CAACCCGTGGAGACGAAGAAATTGGTTTAGAAGACCTAGAAAAACTATTT
ACAGAAGATACAGAAGAAGACGCCGCTGGGTAAGAAGAAAACCTTTTTAC
AAACGTAAAATTAAGAGACTAAATATAGTAGAATGGCAACCTAAATCAAT
TAGAAAATGTAGAATAAAAGGAATGCTATGCTTGTTTCAAACGACAGAAG
ACAGACTGTCATATAACTTTGATATGTATGAAGAGTCTATTATACCAGAA
AAACTGCCGGGAGGGGGGGGATTTAGCATTAAGAATATAAGCTTATATGC
CTTATACCAAGAACACATACATGCACACAACATATTTACACACACAAACA
CAGACAGACCACTAGCAAGATACACAGGCTGTTCTTTAAAATTCTACCAA
AGCAAAGACATAGACTACGTAGTAACATATTCTACATCACTCCCACTAAG
AAGCTCAATGGGAATGTACAACTCCATGCAACCATCCATACATCTAATGC
AACAAAACAAACTAATTGTACCAAGCAAACAAACACAAAAAAGAAGAAAA
CCATATATTAAAAAACATATATCACCACCAACACAAATGAAATCTCAATG
GTACTTTCAACATAACATTGCAAACATACCGCTACTAATGATAAGAACCA
CAGCATTAACATTAGATAATTACTATATAGGAAGCAGACAATTAAGTACA
AATGTCACTATACATACACTTAACACAACATACATCCAAAACAGAGACTG
GGGAGACAGAAATAAAACTTACTACTGCCAAACATTAGGAACACAAAGAT
ACTTCCTATATGGAACACATTCAACTGCACAAAATATTAATGACATAAAG
CTACAAGAACTAATACCTTTAACAAACACACAAGACTATGTACAAGGCTT
TGATTGGACAGAAAAAGACAAACATAACATAACAACCTACAAAGAATTCT
TAACTAAAGGAGCAGGAAATCCATTTCACGCAGAATGGATAACAGCACAA
AACCCAGTAATACACACAGCAAACAGTCCTACACAAATAGAACAAATATA
CACCGCTTCAACAACAACATTCCAAAACAAAAAACTAACAGACCTACCAA
CGCCAGGATATATATTTATAACTCCAACAGTAAGCTTAAGATACAACCCA
TACAAAGACCTAGCAGAAAGAAACAAATGCTACTTTGTAAGAAGCAAAAT
AAATGCACACGGGTGGGACCCAGAACAACACCAAGAATTAATAAACAGTG
ACCTACCACAATGGTTACTATTATTTGGCTACCCAGACTACATAAAAAGA
ACACAAAACTTTGCATTAGTAGACACAAATTACATACTAGTAGACCACTG
CCCATACACAAATCCAGAAAAAACACCATTTATACCTTTAAGCACATCAT
TTATAGAAGGTAGAAGCCCATACAGTCCTTCAGACACACATGAACCAGAT
GAAGAAGACCAAAACAGGTGGTACCCATGCTACCAATATCAACAAGAATC
AATAAATTCAATATGTCTTAGCGGTCCAGGCACACCAAAAATACCAAAAG
GAATAACAGCAGAAGCAAAAGTAAAATATTCCTTTAATTTTAAGTGGGGT
GGTGACCTACCACCAATGTCTACAATTACAAACCCGACAGACCAGCCAAC
ATATGTTGTTCCCAATAACTTCAATGAAACAACTTCGTTACAGAATCCAA
CCACCAGACCAGAGCACTTCTTGTACTCCTTTGACGAAAGGAGGGGACAA
CTTACAGAAAAAGCTACAAAACGCTTGCTTAAAGACTGGGAAACTAAAGA
AACTTCTTTATTGTCTACAGAATACAGATTCGCGGAGCCAACACAAACAC
AAGCCCCACAAGAGGACCCGTCCTCGGAAGAAGAAGAAGAGAGCAACCTC
TTCGAGCGACTCCTCCGACAGCGAACCAAGCAGCTCCAGCTCAAGCGCAG
AATAATACAAACATTGAAAGACCTACAAAAATTAGAATAACTAACAGCAA
AAACACCGTTTACCTATTTCCACCTGAACAAAAGAACAGAAGACTAACAC
CATGGGAAATACAAGAAGACAAAGAAATAGCCAATTTATTTGGCAGACCA
CATAGATACTTTTTAAAAGACATTCCTTTCTATTGGGATATACCCCCAGA
GCCTAAAGTAAACTTTGATTTAAATTTTCAATAAAGAAATAAAGGGCAAG
GCCCCATTAACTCAAAGTCGGTGTCTACCTCTTTAAGTTTAACTTTACTA
AACGGACTCCGCCTCCCTAAATTTGGGCGCCAAAAGGGGGCTCCGCCCCC
TTAAACCCCAGGGGGCTCCGCCCCCTAAAACCCCCAAGGGGGCTACGCCC
CCTTACACCCCC (SEQ ID NO: 1008)

### Annotations:

Putative Domain Base range

### TATA Box 152-158

### Initiation Element 172-187

### Transcriptional Start Site 182

### 5′ UTR Conserved Domain 239-309

### ORF2 343-633

### ORF2/2 343-629; 2196-2505

### ORF2/3 343-629; 2371-2734

### ORF1 522-2540

### ORF1/1 522-629; 2196-2540

### ORF1/2 522-629; 2371-2505

Three open-reading frame region 2276-2502
GC-rich region 2803-2912

TABLE E2
Exemplary Anellovirus amino acid sequences (Betatorquevirus)
Ring 10 (Betatorquevirus)

| | |
|---|---|
| ORF2 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAG (SEQ ID |
| | NO: 1009) |
| ORF2/2 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAGQHMLFPI |
| | TSMKQLRYRIQPPDQSTSCTPLTKGGDNLQKKLQNACLKTGKLKKLLYCLQ |
| | NTDSRSQHKHKPHKRTRPRKKKKRATSSSDSSDSEPSSSSSSAE (SEQ ID NO: |
| | 1010) |
| ORF2/3 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAGIQIRGANT |
| | NTSPTRGPVLGRRRREQPLRATPPTANQAAPAQAQNNTNIERPTKIRITNSKN |
| | TVYLFPPEQKNRRLTPWEIQEDKEIANLFGRPHRYFLKDIPFYWDIPPEPKVN |
| | FDLNFQ (SEQ ID NO: 1011) |
| ORF1 | MPWWYRRRSYNPWRRRNWFRRPRKTIYRRYRRRRRWVRRKPFYKRKIKR |
| | LNIVEWQPKSIRKCRIKGMLCLFQTTEDRLSYNFDMYEESIIPEKLPGGGGFSI |
| | KNISLYALYQEHIHAHNIFTHTNTDRPLARYTGCSLKFYQSKDIDYVVTYSTS |
| | LPLRSSMGMYNSMQPSIHLMQQNKLIVPSKQTQKRRKPYIKKHISPPTQMKS |
| | QWYFQHNIANIPLLMIRTTALTLDNYYIGSRQLSTNVTIHTLNTTYIQNRDW |
| | GDRNKTYYCQTLGTQRYFLYGTHSTAQNINDIKLQELIPLTNTQDYVQGFD |
| | WTEKDKHNITTYKEFLTKGAGNPFHAEWITAQNPVIHTANSPTQIEQIYTAS |
| | TTTFQNKKLTDLPTPGYIFITPTVSLRYNPYKDLAERNKCYFVRSKINAHGW |
| | DPEQHQELINSDLPQWLLLFGYPDYIKRTQNFALVDTNYILVDHCPYTNPEK |
| | TPFIPLSTSFIEGRSPYSPSDTHEPDEEDQNRWYPCYQYQQESINSICLSGPGTP |
| | KIPKGITAEAKVKYSFNFKWGGDLPPMSTITNPTDQPTYVVPNNFNETTSLQ |
| | NPTTRPEHFLYSFDERRGQLTEKATKRLLKDWETKETSLLSTEYRFAEPTQT |
| | QAPQEDPSSEEEEESNLFERLLRQRTKQLQLKRRIIQTLKDLQKLE (SEQ ID |
| | NO: 1012) |
| ORF1/1 | MPWWYRRRSYNPWRRRNWFRRPRKTIYRRYRRRRRWPTYVVPNNFNETT |
| | SLQNPTTRPEHFLYSFDERRGQLTEKATKRLLKDWETKETSLLSTEYRFAEP |
| | TQTQAPQEDPSSEEEEESNLFERLLRQRTKQLQLKRRIIQTLKDLQKLE |
| ORF 1/2 | MPWWYRRRSYNPWRRRNWFRRPRKTIYRRYRRRRRWNTDSRSQHKHKPH |
| | KRTRPRKKKKRATSSSDSSDSEPSSSSSSAE (SEQ ID NO: 1013) |

In some embodiments, an anellovector comprises a nucleic acid comprising a sequence listed in PCT Application No. PCT/US2018/037379, incorporated herein by reference in its entirety. In some embodiments, an anellovector comprises a polypeptide comprising a sequence listed in PCT Application No. PCT/US2018/037379, incorporated herein by reference in its entirety. In some embodiments, an anellovector comprises a nucleic acid comprising a sequence listed in PCT Application No. PCT/US19/65995, incorporated herein by reference in its entirety. In some embodiments, an anellovector comprises a polypeptide comprising a sequence listed in PCT Application No. PCT/US19/65995, incorporated herein by reference in its entirety.

### ORF1 Molecules

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein). In some embodiments, the ORF1 molecule comprises a truncation relative to an Anellovirus ORF1 protein (e.g., an Anellovirus ORF1 protein as described herein). An ORF1 molecule may be capable of binding to other ORF1 molecules, e.g., to form a proteinaceous exterior (e.g., as described herein), e.g., a capsid. In some embodiments, the proteinaceous exterior may enclose a nucleic acid molecule (e.g., a genetic element as described herein). In some embodiments, a plurality of ORF1 molecules may form a multimer, e.g., to form a proteinaceous exterior. In some embodiments, the multimer may be a homomultimer. In other embodiments, the multimer may be a heteromultimer.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

In some embodiments, an ORF1 molecule as described herein comprises one or more lysine-to-histidine mutations relative to a wild-type ORF1 protein sequence (e.g., as described herein). In certain embodiments, the ORF1 molecule comprises one or more lysine-to-histidine mutations in the arginine-rich region and/or the first beta strand.

Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) β-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

N22 Domain

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

Hypervariable Region (HVR)

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

Exemplary ORF1 Sequences

Exemplary Anellovirus ORF1 amino acid sequences, and the sequences of exemplary ORF1 domains, are provided in the tables below. In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z). In some embodiments, an anellovector described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed in any of Tables N-Z), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses (e.g., any combination of ORF1 subsequences selected from the Alphatorquevirus Clade 1-7 subsequences listed in Tables N-Z). In embodiments, the ORF1 molecule comprises one or more of an Arg-rich domain, a jelly-roll domain, an N22 domain, and a CTD from one Anellovirus, and an HVR from another. In embodiments, the ORF1 molecule comprises one or more of a jelly-roll domain, an HVR, an N22 domain, and a CTD from one Anellovirus, and an Arg-rich domain from another. In embodiments, the ORF1 molecule comprises one or more of an Arg-rich domain, an HVR, an N22 domain, and a CTD from one Anellovirus, and a jelly-roll domain from another. In embodiments, the ORF1 molecule comprises one or more of an Arg-rich domain, a jelly-roll domain, an HVR, and a CTD from one Anellovirus, and an N22 domain from another. In embodiments, the ORF1 molecule comprises one or more of an Arg-rich domain, a jelly-roll domain, an HVR, and an N22 domain from one Anellovirus, and a CTD from another.

Additional exemplary Anelloviruses for which the ORF1 molecules, or splice variants or functional fragments thereof, can be utilized in the compositions and methods described herein (e.g., to form the proteinaceous exterior of an anellovector, e.g., by enclosing a genetic element) are described, for example, in PCT Application Nos. PCT/US2018/037379 and PCT/US19/65995 (incorporated herein by reference in their entirety).

TABLE N
Exemplary Anellovirus ORF1 amino acid subsequence
(Alphatorquevirus, Clade 3)

| | |
|---|---|
| Name | Ring 1 |
| Genus/Clade | Alphatorquevirus, Clade 3 |
| Accession Number | AJ620231.1 |
| Protein Accession Number | CAF05750.1 |

Full Sequence: 743 AA
1 10 20 30 40 50
| | | | | |
MAWGWWKRRRRWWFRKRWTRGRLRRRWPRSARRRPRRRRVRRRRRWRRGR
RKTRTYRRRRRFRRRGRKAKL11KLWQPAVIKRCRIKGYIPLIISGNGTF
ATNFTSHINDRIMKGPFGGGHSTMRFSLYILFEEHLRHMNFWTRSNDNLE
LTRYLGASVKIYRHPDQDFIVIYNRRTPLGGNIYTAPSLHPGNAILAKHK
ILVPSLQTRPKGRKAIRLRIAPPTLFTDKWYFQKDIADLTLFNIMAVEAD
LRFPFCSPQTDNTCISFQVLSSVYNNYLSINTFNNDNSDSKLKEFLNKAF
PTTGTKGTSLNALNTFRTEGCISHPQLKKPNPQINKPLESQYFAPLDALW
GDPIYYNDLNENKSLNDIIEKILIKNMITYHAKLREFPNSYQGNKAFCHL
TGIYSPPYLNQGRISPEIFGLYTEIIYNPYTDKGTGNKVWMDPLTKENNI
YKEGQSKCLLTDMPLWTLLFGYTDWCKKDTNNWDLPLNYRLVLICPYTFP
KLYNEKVKDYGYIPYSYKFGAGQMPDGSNYIPFQFRAKWYPTVLHQQQVM
EDISRSGPFAPKVEKPSTQLVMKYCFNFNWGGNPIIEQIVKDPSFQPTYE
IPGTGNIPRRIQVIDPRVLGPHYSFRSWDMRRHTFSRASIKRVSEQQETS
DLVFSGPKKPRVDIPKQETQEESSHSLQRESRPWETEEESETEALSQESQ
EVPFQQQLQQQYQEQLKLRQGIKVLFEQLIRTQQGVHVNPCLR
(SEQ ID NO: 185)

### Annotations:

Putative Domain AA range

### Arg-Rich Region 1-68

Jelly-roll domain 69-280

### Hypervariable Region 281-413

### N22 414-579

### C-terminal Domain 580-743

TABLE O
Exemplary Anellovirus ORF1 amino acid subsequence (Alphatorquevirus, Clade 3)

Ring1 ORF1 (Alphatorquevirus Clade 3)

| | |
|---|---|
| Arg-Rich | MAWGWWKRRRRWWFRKRWTRGRLRRRWPRSARRRPRRRRVRRRRRWRR |
| Region | GRRKTRTYRRRRRFRRRGRK (SEQ ID NO: 186) |
| Jelly-roll | AKLIIKLWQPAVIKRCRIKGYIPLIISGNGTFATNFTSHINDRIMKGPFGGGHST |
| Domain | MRFSLYILFEEHLRHMNFWTRSNDNLELTRYLGASVKIYRHPDQDFIVIYNRR |
| | TPLGGNIYTAPSLHPGNAILAKHKILVPSLQTRPKGRKAIRLRIAPPTLFTDKWY |
| | FQKDIADLTLFNIMAVEADLRFPFCSPQTDNTCISFQVLSSVYNNYLSI (SEQ ID |
| | NO: 187) |
| Hypervariable | NTFNNDNSDSKLKEFLNKAFPTTGTKGTSLNALNTFRTEGCISHPQLKKPNPQI |
| domain | NKPLESQYFAPLDALWGDPIYYNDLNENKSLNDIIEKILIKNMITYHAKLREFP |
| | NSYQGNKAFCHLTGIYSPPYLNQGR (SEQ ID NO: 188) |
| N22 | ISPEIFGLYTEIIYNPYTDKGTGNKVWMDPLTKENNIYKEGQSKCLLTDMPLW |
| | TLLFGYTDWCKKDTNNWDLPLNYRLVLICPYTFPKLYNEKVKDYGYIPYSYK |
| | FGAGQMPDGSNYIPFQFRAKWYPTVLHQQQVMEDISRSGPFAPKVEKPSTQL |
| | VMKYCFNFN (SEQ ID NO: 189) |
| C-terminal | WGGNPIIEQIVKDPSFQPTYEIPGTGNIPRRIQVIDPRVLGPHYSFRSWDMRRHT |
| domain | FSRASIKRVSEQQETSDLVFSGPKKPRVDIPKQETQEESSHSLQRESRPWETEEE |
| | SETEALSQESQEVPFQQQLQQQYQEQLKLRQGIKVLFEQLIRTQQGVHVNPCL |
| | R (SEQ ID NO: 190) |

TABLE P
Exemplary Anellovirus ORF1 amino acid subsequence (Betatorquevirus)

| | |
|---|---|
| Name | Ring 2 |
| Genus/Clade | Betatorquevirus |
| Accession Number | JX134045.1 |
| Protein Accession Number | AGG91484.1 |

Full Sequence: 666 AA
1 10 20 30 40 50
| | | | | |
MPYYYRRRRYNYRRPRWYGRGWIRRPFRRRFRRKRRVRPTYTTIPLKQWQ
PPYKRTCYIKGQDCLIYYSNLRLGMNSTMYEKSIVPVHWPGGGSFSVSML
TLDALYDIHKLCRNWWTSTNQDLPLVRYKGCKITFYQSTFTDYIVRIHTE
LPANSNKLTYPNTHPLMMMMSKYKHIIPSRQTRRKKKPYTKIFVKPPPQF
ENKWYFATDLYKIPLLQIHCTACNLQNPFVKPDKLSNNVTLWSLNTISIQ
NRNMSVDQGQSWPFKILGTQSFYFYFYTGANLPGDTTQIPVADLLPLTNP
RINRPGQSLNEAKITDHITFTEYKNKFTNYWGNPFNKHIQEHLDMILYSL
KSPEAIKNEWTTENMKWNQLNNAGTMALTPFNEPIFTQIQYNPDRDTGED
TQLYLLSNATGTGWDPPGIPELILEGFPLWLIYWGFADFQKNLKKVTNID
TNYMLVAKTKFTQKPGTFYLVILNDTFVEGNSPYEKQPLPEDNIKWYPQV
QYQLEAQNKLLQTGPFTPNIQGQLSDNISMFYKFYFKWGGSPPKAINVEN
PAHQIQYPIPRNEHETTSLQSPGEAPESILYSFDYRHGNYTTTALSRISQ
DWALKDTVSKITEPDRQQLLKQALECLQISEETQEKKEKEVQQLISNLRQ
QQQLYRERIISLLKDQ (SEQ ID NO: 215)

### Annotations:

Putative Domain AA range

### Arg-Rich Region 1-38

Jelly-roll domain 39-246

### Hypervariable Region 247-374

### N22 375-537

### C-terminal Domain 538-666

TABLE Q
Exemplary Anellovirus ORF1 amino acid subsequence (Betatorquevirus)
Ring2 ORF1 (Betatorquevirus)

| | |
|---|---|
| Arg-Rich | MPYYYRRRRYNYRRPRWYGRGWIRRPFRRRFRRKRRVR (SEQ ID NO: 216) |
| Region | |
| Jelly-roll | PTYTTIPLKQWQPPYKRTCYIKGQDCLIYYSNLRLGMNSTMYEKSIVPVHWPG |
| Domain | GGSFSVSMLTLDALYDIHKLCRNWWTSTNQDLPLVRYKGCKITFYQSTFTDYI |
| | VRIHTELPANSNKLTYPNTHPLMMMMSKYKHIIPSRQTRRKKKPYTKIFVKPP |
| | PQFENKWYFATDLYKIPLLQIHCTACNLQNPFVKPDKLSNNVTLWSLNT (SEQ |
| | ID NO: 217) |
| Hypervariable | ISIQNRNMSVDQGQSWPFKILGTQSFYFYFYTGANLPGDTTQIPVADLLPLTNP |
| domain | RINRPGQSLNEAKITDHITFTEYKNKFTNYWGNPFNKHIQEHLDMILYSLKSPE |
| | AIKNEWTTENMKWNQLNNAG (SEQ ID NO: 218) |
| N22 | TMALTPFNEPIFTQIQYNPDRDTGEDTQLYLLSNATGTGWDPPGIPELILEGFPL |
| | WLIYWGFADFQKNLKKVTNIDTNYMLVAKTKFTQKPGTFYLVILNDTFVEGN |
| | SPYEKQPLPEDNIKWYPQVQYQLEAQNKLLQTGPFTPNIQGQLSDNISMFYKF |
| | YFK (SEQ ID NO: 219) |
| C-terminal | WGGSPPKAINVENPAHQIQYPIPRNEHETTSLQSPGEAPESILYSFDYRHGNYT |
| domain | TTALSRISQDWALKDTVSKITEPDRQQLLKQALECLQISEETQEKKEKEVQQLI |
| | SNLRQQQQLYRERIISLLKDQ (SEQ ID NO: 220) |

TABLE R
Exemplary Anellovirus ORF1 amino acid subsequence (Gammatorquevirus)

| | |
|---|---|
| Name | Ring 4 |
| Genus/Clade | Gammatorquevirus |
| Accession Number | |
| Protein Accession Number | |

Full Sequence: 662 AA
1 10 20 30 40 50
| | | | | |
MPFWWRRRRKFWTNNRFNYTKRRRYRKRWPRRRRRRRPYRRPVRRRRRKL
RKVKRKKKSLIVRQWQPDSIRTCKIIGQSAIWGAEGKQMYCYTVNKLIN
VPPKTPYGGGFGVDQYTLKYLYEEYRFAQNIWTQSNVLKDLCRYINVKLI
FYRDNKTDFVLSYDRNPPFQLTKFTYPGAHPQQIMLQKHHKFILSQMTKP
NGRLTKKLKIKPPKQMLSKWFFSKQFCKYPLLSLKASALDLRHSYLGCCN
ENPQVFFYYLNHGYYTITNWGAQSSTAYRPNSKVTDTTYYRYKNDRKNIN
IKSHEYEKSISYENGYFQSSFLQTQCIYTSERGEACIAEKPLGIAIYNPV
KDNGDGNMIYLVSTLANTWDQPPKDSAILIQGVPIWLGLFGYLDYCRQIK
ADKTWLDSHVLVIQSPAIFTYPNPGAGKWYCPLSQSFINGNGPFNQPPTL
LQKAKWFPQIQYQQEIINSFVESGPFVPKYANQTESNWELKYKYVFTFKW
GGPQFHEPEIADPSKQEQYDVPDTFYQTIQIEDPEGQDPRSLIHDWDYRR
GFIKERSLKRMSTYFSTHTDQQATSEEDIPKKKKRIGPQLTVPQQKEEET
LSCLLSLCKKDTFQETETQEDLQQLIKQQQEQQLLLKRNILQLIHKLKEN
QQMLQLHTGMLP (SEQ ID NO: 925)

### Annotations:

Putative Domain AA range

### Arg-Rich Region 1-58

Jelly-roll domain 59-260

### Hypervariable Region 261-339

### N22 340-499

### C-terminal Domain 500-662

TABLE S
Exemplary Anellovirus ORF1 amino acid subsequence (Gammatorquevirus)
Ring4 (Gammatorquevirus)

| | |
|---|---|
| Arg-Rich | MPFWWRRRRKFWTNNRFNYTKRRRYRKRWPRRRRRRRPYRRPVRRRRRKL |
| Region | RKVKRKKK (SEQ ID NO: 926) |
| Jelly-roll | SLIVRQWQPDSIRTCKIIGQSAIVVGAEGKQMYCYTVNKLINVPPKTPYGGGF |
| Domain | GVDQYTLKYLYEEYRFAQNIWTQSNVLKDLCRYINVKLIFYRDNKTDFVLSY |
| | DRNPPFQLTKFTYPGAHPQQIMLQKHHKFILSQMTKPNGRLTKKLKIKPPKQM |
| | LSKWFFSKQFCKYPLLSLKASALDLRHSYLGCCNENPQVFFYYL (SEQ ID NO: |
| | 927) |
| Hypervariable | NHGYYTITNWGAQSSTAYRPNSKVTDTTYYRYKNDRKNINIKSHEYEKSISYE |
| domain | NGYFQSSFLQTQCIYTSERGEACIAE (SEQ ID NO: 928) |
| N22 | KPLGIAIYNPVKDNGDGNMIYLVSTLANTWDQPPKDSAILIQGVPIWLGLFGY |
| | LDYCRQIKADKTWLDSHVLVIQSPAIFTYPNPGAGKWYCPLSQSFINGNGPFN |
| | QPPTLLQKAKWFPQIQYQQEIINSFVESGPFVPKYANQTESNWELKYKYVFTF |
| | K (SEQ ID NO: 929) |
| C-terminal | WGGPQFHEPEIADPSKQEQYDVPDTFYQTIQIEDPEGQDPRSLIHDWDYRRGFI |
| domain | KERSLKRMSTYFSTHTDQQATSEEDIPKKKKRIGPQLTVPQQKEEETLSCLLSL |
| | CKKDTFQETETQEDLQQLIKQQQEQQLLLKRNILQLIHKLKENQQMLQLHTG |
| | MLP (SEQ ID NO: 930) |

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization signal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34 of PCT/US19/65995). In some embodiments, the conserved motifs may show 60, 70, 80, 85, 90, 95, or 100% sequence identity to an ORF1 protein of one or more wild-type Anellovirus clades (e.g., Alphatorquevirus, clade 1; Alphatorquevirus, clade 2; Alphatorquevirus, clade 3; Alphatorquevirus, clade 4; Alphatorquevirus, clade 5; Alphatorquevirus, clade 6; Alphatorquevirus, clade 7; Betatorquevirus; and/or Gammatorquevirus). In embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule or a nucleic acid molecule encoding same comprises at least one difference (e.g., a mutation, chemical modification, or epigenetic alteration) relative to a wild-type ORF1 protein, e.g., as described herein.

Conserved ORF1 Motif in N22 Domain

In some embodiments, a polypeptide (e.g., an ORF1 molecule) described herein comprises the amino acid sequence YNPX²DXGX²N (SEQ ID NO: 829), wherein Xⁿ is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

Exemplary YNPX²DXGX²N (SEQ ID NO: 829) motif-flanking secondary structures are described in Example 47 and FIG. 48 of PCT/US19/65995; incorporated herein by reference in its entirety. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995, flanking a YNPX²DXGX²N (SEQ ID NO: 829) motif (e.g., as described herein).

Conserved Secondary Structural Motif in ORF1 Jelly-Roll Domain

In some embodiments, a polypeptide (e.g., an ORF1 molecule) described herein comprises one or more secondary structural elements comprised by an Anellovirus ORF1 protein (e.g., as described herein). In some embodiments, an ORF1 molecule comprises one or more secondary structural elements comprised by the jelly-roll domain of an Anellovius ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

Exemplary jelly-roll domain secondary structures are described in Example 47 and FIG. 47 of PCT/US19/65995. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 47 of PCT/US19/65995.

Consensus ORF1 Domain Sequences

In some embodiments, an ORF1 molecule, e.g., as described herein, comprises one or more of a jelly-roll domain, N22 domain, and/or C-terminal domain (CTD). In some embodiments, the jelly-roll domain comprises an amino acid sequence having a jelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(X_{a-b})” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYATHS | 227 |
| | DDTIQKGPFGGGMSTTTFSLRVLYDEYQRFMNRWTYSNED | |
| | LDLARYLGCKFTFYRHPDXDFIVQYNTNPPFKDTKLTAPSIH | |
| | P(X₁₋₅)GMLMLSKRKILIPSLKTRPKGKHYVKVRIGPPKLFED | |
| | KWYTQSDLCDVPLVXLYATAADLQHPFGSPQTDNPCVTFQ | |
| | VLGSXYNKHLSISP; | |
| | wherein X = any amino acid. | |
| N22 | SNFEFPGAYTDITYNPLTDKGVGNMVWIQYLTKPDTIXDKT | 228 |
| | QS(X₀₋₃)KCLIEDLPLWAALYGYVDFCEKETGDSAIIXNXGRV | |
| | LIRCPYTKPPLYDKT(X₀₋₄)NKGFVPYSTNFGNGKMPGGSGY | |
| | VPIYWRARWYPTLFHQKEVLEDIVQSGPFAYKDEKPSTQLV | |
| | MKYCFNFN; | |
| | wherein X = any amino acid. | |
| CTD | WGGNPISQQVVRNPCKDSG(X₀₋₃)SGXGRQPRSVQVVDPKY | 229 |
| | MGPEYTFHSWDWRRGLFGEKAIKRMSEQPTDDEIFTGGXPK | |
| | RPRRDPPTXQXPEE(X₁₋₄)QKESSSFR(X₂₋₁₄)PWESSSQEXESES | |
| | QEEEE(X₀₋₃₀)EQTVQQQLRQQLREQRRLRVQLQLLFQQLLKT | |
| | (X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESIVPH | 230 |
| | HEPGGGGWSIQQFTLGALYEEHLKLRNWWTKSNDGLPLVR | |
| | YLGCTIKLYRSEDTDYIVTYQRCYPMTATKLTYLSTQPSRM | |
| | LMNKHKIIVPSKXT(X₁₋₄)NKKKKPYKKIFIKPPSQMQNKWYF | |
| | QQDIANTPLLQLTXTACSLDRMYLSSDSISNNITFTSLNTNFF | |
| | QNPNFQ; | |
| | wherein X = any amino acid. | |
| N22 | (X₄₋₁₀)TPLYFECRYNPFKDKGTGNKVYLVSNN(X₁₋₈)TGWDPP | 231 |
| | TDPDLIIEGFPLWLLLWGWLDWQKKLGKIQNIDTDYILVIQS | |
| | XYYIPP(X₁₋₃)KLPYYVPLDXD(X₀₋₂)FLHGRSPY(X₃₋₁₆)PSDKQH | |
| | WHPKVRFQXETINNIALTGPGTPKLPNQKSIQAHMKYKFYF | |
| | K; | |
| | wherein X = any amino acid. | |
| CTD | WGGCPAPMETITDPCKQPKYPIPNNLLQTTSLQXPTTPIETYL | 232 |
| | YKFDERRGLLTKKAAKRIKKDXTTETTLFTDTGXXTSTTLPT | |
| | XXQTETTQEEXTSEEE(X₀₋₅)ETLLQQLQQLRRKQKQLRXRIL | |
| | QLLQLLXLL(X₀₋₂₆)*; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNEKDE | 233 |
| | YTPPKAPGGGGFGVELFSLEYLYEQWKARNNIWTKSNXYK | |
| | DLCRYTGCKITFYRHPTTDFIVXYSRQPPFEIDKXTYMXXHP | |
| | QXLLLRKHKKIILSKATNPKGKLKKKIKIKPPKQMLNKWFF | |
| | QKQFAXYGLVQLQAAACBLRYPRLGCCNENRLITLYYLN; | |
| | wherein X = any amino acid. | |
| N22 | LPIVVARYNPAXDTGKGNKXWLXSTLNGSXWAPPTTDKDL | 234 |
| | IIEGLPLWLALYGYWSYJKKVKKDKGILQSHMFVVKSPAIQP | |
| | LXTATTQXTFYPXIDNSFIQGKXPYDEPJTXNQKKLWYPTLE | |
| | HQQETINAIVESGPYVPKLDNQKNSTWELXYXYTFYFK; | |
| | wherein X = any amino acid. | |
| CTD | WGGPQIPDQPVEDPKXQGTYPVPDTXQQTIQIXNPLKQKPE | 235 |
| | TMFHDWDYRRGIITSTALKRMQENLETDSSFXSDSEETP(X₀₋₂) | |
| | KKKKRLTXELPXPQEETEEIQSCLLSLCEESTCQEE(X₁₋₆)ENL | |
| | QQLIHQQQQQQQQLKHNILKLLSDLKZKQRLLQLQTGILE | |
| | (X₁₋₁₀)*; | |
| | wherein X = any amino acid. | |

In some embodiments, the jelly-roll domain comprises a jelly-roll domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto.

Identification of ORF1 Protein Sequences

In some embodiments, an Anellovirus ORF1 protein sequence, or a nucleic acid sequence encoding an ORF1 protein, can be identified from the genome of an Anellovirus (e.g., a putative Anellovirus genome identified, for example, by nucleic acid sequencing techniques, e.g., deep sequencing techniques). In some embodiments, an ORF1 protein sequence is identified by one or more (e.g., 1, 2, or all 3) of the following selection criteria:

(i) Length Selection: Protein sequences (e.g., putative Anellovirus ORF1 sequences passing the criteria described in (ii) or (iii) below) may be size-selected for those greater than about 600 amino acid residues to identify putative Anellovirus ORF1 proteins. In some embodiments, an Anellovirus ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an Anellovirus ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.

(h) Presence of ORF1 motif Protein sequences (e.g., putative Anellovirus ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN. In some embodiments, a putative Anellovirus ORF1 sequence comprises the sequence Y[NCS]XXDX[GASKR]XX[NTSVAK].

(iii) Presence of arginine-rich region: Protein sequences (e.g., putative Anellovirus ORF1 sequences passing the criteria described in (i) and/or (ii) above) may be filtered for those that include an arginine-rich region (e.g., as described herein). In some embodiments, a putative Anellovirus ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative Anellovirus ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative Anellovirus ORF1 protein. In some embodiments, the arginine-rich region is positioned at least about 50 amino acids downstream of the start codon of the putative Anellovirus ORF1 protein.

### ORF2 Molecules

In some embodiments, the anellovector comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in any of Tables A2, A4, A6, A8, A10, A12, C1-C5, 2, 4, 6, 8, 10, 12, 14, 16, or 18), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in any of Tables A2, A4, A6, A8, A10, A12, C1-C5, 2, 4, 6, 8, 10, 12, 14, 16, or 18.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule or a nucleic acid molecule encoding same comprises at least one difference (e.g., a mutation, chemical modification, or epigenetic alteration) relative to a wild-type ORF2 protein, e.g., as described herein (e.g., as shown in any of Tables A2, A4, A6, A8, A10, A12, C1-C5, 2, 4, 6, 8, 10, 12, 14, 16, or 18).

Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein Xⁿ is a contiguous sequence of any n amino acids. In embodiments, X⁷ indicates a contiguous sequence of any seven amino acids. In embodiments, X³ indicates a contiguous sequence of any three amino acids. In embodiments, X¹ indicates any single amino acid. In embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein Xⁿ is a contiguous sequence of any n amino acids.

### Genetic Elements

In some embodiments, the anellovector comprises a genetic element. In some embodiments, the genetic element has one or more of the following characteristics: is substantially non-integrating with a host cell's genome, is an episomal nucleic acid, is a single stranded RNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence.

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb. In some embodiments, the genetic element has a length between about 3.6-3.9 kb. In some embodiments, the genetic element has a length between about 2.8-2.9 kb. In some embodiments, the genetic element has a length between about 2.0-3.2 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences.

In embodiments, the genetic element was produced from a double-stranded circular DNA (e.g., produced by transcription).

In some embodiments, the genetic element does not comprise one or more bacterial plasmid elements (e.g., a bacterial origin of replication or a selectable marker, e.g., a bacterial resistance gene). In some embodiments, the genetic element does not comprise a bacterial plasmid backbone.

In one embodiment, the disclosure provides a genetic element comprising a nucleic acid sequence (e.g., an RNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

### Protein Binding Sequence

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8 of PCT/US19/65995.

In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence, e.g., as described herein.

In embodiments, the protein binding sequence has at least about 70%, 75%, 80%, 85%, 90%, 95%, 96%, 97%, 98%, 99%, or 100% sequence identity to an Anellovirus 5′ UTR conserved domain nucleotide sequence, e.g., as described herein.

### 5′ UTR Regions

In some embodiments, a nucleic acid molecule as described herein (e.g., a genetic element, genetic element construct, or genetic element region) comprises a 5′ UTR sequence, e.g., a 5′ UTR conserved domain sequence as described herein (e.g., in any of Tables A1, B1, or C1), or a sequence having at least 75%, 80%, 85%, 90%, 95%, 96%, 97%, 98%, or 99% sequence identity thereto.

In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having no more than 1, 2, 3, 4, 5, 6, 7, 8, 9, or 10 nucleotide differences (e.g., substitutions, deletions, or additions) relative thereto. In embodiments, X₁ is A. In embodiments, X₁ is absent.

In some embodiments, the 5′ UTR sequence comprises the nucleic acid sequence of the 5′ UTR of an Alphatorquevirus (e.g., Ring1), or a sequence having at least 75%, 80%, 85%, 90%, 95%, 96%, 97%, 98%, or 99% sequence identity thereto. In embodiments, the 5′ UTR sequence comprises the nucleic acid sequence of the 5′ UTR conserved domain listed in Table A1, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR conserved domain listed in Table A1. In some embodiments, the nucleic acid molecule comprises a nucleic acid sequence having at least 95.775% sequence identity to the 5′ UTR conserved domain listed in Table A1. In some embodiments, the nucleic acid molecule comprises a nucleic acid sequence having at least 97% sequence identity to the 5′ UTR conserved domain listed in Table A1. In some embodiments, the nucleic acid molecule comprises a nucleic acid sequence having at least 97.183% sequence identity to the 5′ UTR conserved domain listed in Table A1. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of an Betatorquevirus (e.g., Ring2), or a sequence having at least 75%, 80%, 85%, 86%, 87%, 88%, 89%, 90%, 91%, 92%, 93%, 94%, 95%, 96%, 97%, 98%, or 99% sequence identity thereto. In embodiments, the 5′ UTR sequence comprises the nucleic acid sequence of the 5′ UTR conserved domain listed in Table B1, or a sequence having at least 75%, 80%, 85%, 86%, 87%, 88%, 89%, 90%, 91%, 92%, 93%, 94%, 95%, 96%, 97%, 98%, or 99% sequence identity thereto. In some embodiments, the nucleic acid molecule comprises a nucleic acid sequence having at least 85% sequence identity to the 5′ UTR conserved domain listed in Table B1. In some embodiments, the nucleic acid molecule comprises a nucleic acid sequence having at least 87% sequence identity to the 5′ UTR conserved domain listed in Table B1. In some embodiments, the nucleic acid molecule comprises a nucleic acid sequence having at least 87.324% sequence identity to the 5′ UTR conserved domain listed in Table B1. In some embodiments, the nucleic acid molecule comprises a nucleic acid sequence having at least 88% sequence identity to the 5′ UTR conserved domain listed in Table B1. In some embodiments, the nucleic acid molecule comprises a nucleic acid sequence having at least 88.732% sequence identity to the 5′ UTR conserved domain listed in Table B1. In some embodiments, the nucleic acid molecule comprises a nucleic acid sequence having at least 91% sequence identity to the 5′ UTR conserved domain listed in Table B1. In some embodiments, the nucleic acid molecule comprises a nucleic acid sequence having at least 91.549% sequence identity to the 5′ UTR conserved domain listed in Table B1. In some embodiments, the nucleic acid molecule comprises a nucleic acid sequence having at least 92% sequence identity to the 5′ UTR conserved domain listed in Table B1. In some embodiments, the nucleic acid molecule comprises a nucleic acid sequence having at least 92.958% sequence identity to the 5′ UTR conserved domain listed in Table B1. In some embodiments, the nucleic acid molecule comprises a nucleic acid sequence having at least 94% sequence identity to the 5′ UTR conserved domain listed in Table B1. In some embodiments, the nucleic acid molecule comprises a nucleic acid sequence having at least 94.366% sequence identity to the 5′ UTR conserved domain listed in Table B1. In some embodiments, the nucleic acid molecule comprises a nucleic acid sequence having at least 95% sequence identity to the 5′ UTR conserved domain listed in Table B1. In some embodiments, the nucleic acid molecule comprises a nucleic acid sequence having at least 95.775% sequence identity to the 5′ UTR conserved domain listed in Table B1. In some embodiments, the nucleic acid molecule comprises a nucleic acid sequence having at least 97% sequence identity to the 5′ UTR conserved domain listed in Table B1. In some embodiments, the nucleic acid molecule comprises a nucleic acid sequence having at least 97.183% sequence identity to the 5′ UTR conserved domain listed in Table B1. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of a Gammatorquevirus (e.g., Ring4), or a sequence having at least 75%, 80%, 85%, 90%, 95%, 96%, 97%, 98%, or 99% sequence identity thereto. In embodiments, the 5′ UTR sequence comprises the nucleic acid sequence of the 5′ UTR conserved domain listed in Table C1, or a sequence having at least 75%, 80%, 85%, 90%, 95%, 96%, 97%, 98%, or 99% sequence identity thereto. In some embodiments, the nucleic acid molecule comprises a nucleic acid sequence having at least 97% sequence identity to the 5′ UTR conserved domain listed in Table C1. In some embodiments, the nucleic acid molecule comprises a nucleic acid sequence having at least 97.183% sequence identity to the 5′ UTR conserved domain listed in Table C1. In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having at least 85%, 90%, 95%, 96%, 97%, 98%, or 99% sequence identity thereto.

In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Anellovirus 5′ UTR sequence, e.g., a nucleic acid sequence shown in Table 38. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

TABLE 38
Exemplary 5’ UTR sequences from Anelloviruses

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = G or A | |
| | X₄ = T or C | |
| | X₅ = A, C, or T | |
| Exemplary TTV | CGGGTGCCGGAGGTGAGTTTACACACCGCAGTC | 106 |
| Sequence | AAGGGGCAATTCGGGCTCGGGACTGGCCGGGCT | |
| | WTGGG | |
| TTV-CT30F | CGGGTGCCGTAGGTGAGTTTACACACCGCAGTC | 107 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 108 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 109 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 110 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | ill |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 112 |
| Consensus 5 ’ UTR | AAGGGGCAATTCGGGCTCGGGACTGGCCGGGC | |
| | X₁X₂TGGG; wherein X₁ comprises T or C, | |
| | and wherein X₂ comprises A, C, or T. | |
| Alphatorquevirus | CGGGTGCCGTAGGTGAGTTTACACACCGCAGTC | 113 |
| Clade 1 5’ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 114 |
| Clade 2 5’ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 115 |
| Clade 3 5’ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 116 |
| Clade 4 5’ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 117 |
| Clade 5 5’ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 118 |
| Clade 6 5’ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 119 |
| Clade 7 5’UTR (e.g., | AAGGGGCAATTCGGGCTCGGGACTGGCCGGGCT | |
| TTV-HD16d) | ATGGG | |

Identification of 5′ UTR Sequences

In some embodiments, an Anellovirus 5′ UTR sequence can be identified within the genome of an Anellovirus (e.g., a putative Anellovirus genome identified, for example, by nucleic acid sequencing techniques, e.g., deep sequencing techniques). In some embodiments, an Anellovirus 5′ UTR sequence is identified by one or both of the following steps:

(i) Identification of circularization junction point: In some embodiments, a 5′ UTR will be positioned near a circularization junction point of a full-length, circularized Anellovirus genome. A circularization junction point can be identified, for example, by identifying overlapping regions of the sequence. In some embodiments, a overlapping region of the sequence can be trimmed from the sequence to produce a full-length Anellovirus genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anellovirus genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anellovirus, e.g., as described herein).

(ii) Identification of 5′ UTR sequence: Once a putative Anellovirus genome sequence has been obtained, the sequence (or portions thereof, e.g., having a length between about 40-50, 50-60, 60-70, 70-80, 80-90, or 90-100 nucleotides) can be compared to one or more Anellovirus 5′ UTR sequences (e.g., as described herein) to identify sequences having substantial homology thereto. In some embodiments, a putative Anellovirus 5′ UTR region has at least 50%, 60%, 70%, 75%, 80%, 85%, 90%, 95%, 96%, 97%, 98%, 99%, or 100% sequence identity to an Anellovirus 5′ UTR sequence as described herein.

### GC-Rich Regions

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39. In embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39.

In embodiments, the 36-nucleotide GC-rich sequence is selected from:

(i)
(SEQ ID NO: 160)
CGCGCTGCGCGCGCCGCCCAGTAGGGGGAGCCATGC,
(ii)
(SEQ ID NO: 164)
GCGCTX₁CGCGCGCGCGCCGGGGGGCTGCGCCCCCCC
wherein X₁ is selected from T, G, or A;
(iii)
(SEQ ID NO: 165)
GCGCTTCGCGCGCCGCCCACTAGGGGGCGTTGCGCG;
(iv)
(SEQ ID NO: 166)
GCGCTGCGCGCGCCGCCCAGTAGGGGGCGCAATGCG;
(v)
(SEQ ID NO: 167)
GCGCTGCGCGCGCGGCCCCCGGGGGAGGCATTGCCT;
(vi)
(SEQ ID NO: 168)
GCGCTGCGCGCGCGCGCCGGGGGGGCGCCAGCGCCC;
(vii)
(SEQ ID NO: 169)
GCGCTTCGCGCGCGCGCCGGGGGGCTCCGCCCCCCC;
(viii)
(SEQ ID NO: 170)
GCGCTTCGCGCGCGCGCCGGGGGGCTGCGCCCCCCC;
(ix)
(SEQ ID NO: 171)
GCGCTACGCGCGCGCGCCGGGGGGCTGCGCCCCCCC;
or
(x)
(SEQ ID NO: 172)
GCGCTACGCGCGCGCGCCGGGGGGCTCTGCCCCCCC

In embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CGCGC | 120 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X₃₃ | |
| | | GGGGGGCTCCGX₃₄CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 121 |
| Sequence | | DCGCGCGCSNNNCRCCRGGGGGNNNNCWG | |
| | | CSNCNCCCCCCCCCGCGCATGCGCGGGKCC | |
| | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 122 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 123 |
| | | CG | |
| | Fragment 3 | CCCCCCGGCCCCCCCCCGTGCTAAACCCAC | 124 |
| | | CGCGCATGCGCGACCACGCCCCCGCCGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 126 |
| | Fragment 2 | GGGGGCG | 127 |
| | Fragment 3 | GCCGCG | 128 |
| | Fragment 4 | TTCGCGCGCCGCCCACCAGGGGGTG | 129 |
| | Fragment 5 | CTGCG | 130 |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCCCCC | 133 |
| | | GTGCTAAACCCACCGCGCATGCGCGACCAC | |
| | | GCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | GCCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGCGCCAGCG | 137 |
| | Fragment 4 | CCCCCCCCCCCGCGCAT | 138 |
| | Fragment 5 | GCACGGGTCCCCCCCCCCACGGGGGGCTCC | 139 |
| | | G | |
| | Fragment 6 | CCCCCCGGCCCCCCCCC | 140 |
| TTV-JA20 | Full sequence | CCGTCGGCGGGGGGGCCGCGCGCTGCGCG | 141 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 142 |
| | | CGCGGCCC | |
| | Fragment 2 | CCGGGGGAGGCACAGCCTCCCCCCCCCGCG | 143 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG- | 144 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | 148 |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTAGG | |
| | | GGGAGCCATGCG---CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTAGG | 155 |
| | | GGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGGGA | 160 |
| | | GCCATGC | |
| | Fragment 8 | CCGCCATCTTAAGTAGTTGAGGCGGACGGT | 161 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 162 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional | 36-nucleotide | CGCGCTGCGCGCGCCGCCCAGTAGGGGGA | 163 |
| GC-rich | consensus GC- | GCCATGC | |
| Sequences | rich region | | |
| | sequence 1 | | |
| | 36-nucleotide | GCGCTX1CGCGCGCGCGCCGGGGGGCTGCG | 164 |
| | region | CCCCCCC, wherein X) is selected | |
| | consensus | from T, G, or A | |
| | sequence 2 | | |
| | TTV Clade 1 | GCGCTTCGCGCGCCGCCCACTAGGGGGCGT | 165 |
| | 36-nucleotide | TGCGCG | |
| | region | | |
| | TTV Clade 3 | GCGCTGCGCGCGCCGCCCAGTAGGGGGCG | 166 |
| | 36-nucleotide | CAATGCG | |
| | region | | |
| | TTV Clade 3 | GCGCTGCGCGCGCGGCCCCCGGGGGAGGC | 167 |
| | isolate GH1 36- | ATTGCCT | |
| | nucleotide | | |
| | region | | |
| | TTV Clade 3 | GCGCTGCGCGCGCGCGCCGGGGGGGCGCC | 168 |
| | slel932 36- | AGCGCCC | |
| | nucleotide | | |
| | region | | |
| | TTV Clade 4 | GCGCTTCGCGCGCGCGCCGGGGGGCTCCGC | 169 |
| | ctdc002 36- | CCCCCC | |
| | nucleotide | | |
| | region | | |
| | TTV Clade 5 | GCGCTTCGCGCGCGCGCCGGGGGGCTGCGC | 170 |
| | 36-nucleotide | CCCCCC | |
| | region | | |
| | TTV Clade 6 | GCGCTACGCGCGCGCGCCGGGGGGCTGCG | 171 |
| | 36-nucleotide | CCCCCCC | |
| | region | | |
| | TTV Clade 7 | GCGCTACGCGCGCGCGCCGGGGGGCTCTGC | 172 |
| | 36-nucleotide | CCCCCC | |
| | region | | |
| Additional | TTV-CT30F | GCGGCGGGGGGGCGGCCGCGTTCGCGCGC | 801 |
| sequences | | CGCCCACCAGGGGGTGCTGCGCGCCCCCCC | |
| GC-rich region | | CCGCGCATGCGCGGGGCCCCCCCCCGGGG | |
| Alphatorquevirus | | GGGCTCCGCCCCCCCGGCCCCCCCCCGTGC | |
| | | TAAACCCACCGCGCATGCGCGACCACGCCC | |
| | | CCGCCGCC | |
| | TTV-P13-1 | CCGAGCGTTAGCGAGGAGTGCGACCCTACC | 802 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 803 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 804 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 805 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 806 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 807 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

### Effectors

In some embodiments, the genetic element may include one or more sequences that are or encode an effector, e.g., a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described herein.

Regulatory Nucleic Acids

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In embodiments, the regulatory nucleic acid encodes an miRNA. In some embodiments, the regulatory nucleic acid is endogenous to a wild-type Anellovirus. In some embodiments, the regulatory nucleic acid is exogenous to a wild-type Anellovirus.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599, 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polypeptide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

Exemplary Secreted Polypeptide Effectors

Exemplary secreted therapeutics are described herein, e.g., in the tables below.

TABLE 50
Exemplary cytokines and cytokine receptors

| | | Entrez | |
|---|---|---|---|
| Cytokine | Cytokine receptor(s) | Gene ID | UniProt ID |
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD 131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gpl30 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| e | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/ IL-20R2 | | |
| IL-25 | IL-17RAand IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e g., p35, EBI3, or | IL-12Rβ2/gp130; IL- | 10148 | Q14213 |
| a heterodimer thereof) | 12Rβ2/IL-12Rβ2; | | |
| | gpl30/gpl30 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

In some embodiments, an effector described herein comprises a cytokine of Table 50, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 50 or a functional variant or fragment thereof) and a second, heterologous region. In some embodiments, the first region is a first cytokine polypeptide of Table 50. In some embodiments, the second region is a second cytokine polypeptide of Table 50, wherein the first and second cytokine polypeptides form a cytokine heterodimer with each other in a wild-type cell. In some embodiments, the polypeptide of Table 50 or functional variant thereof comprises a signal sequence, e.g., a signal sequence that is endogenous to the effector, or a heterologous signal sequence. In some embodiments, an anellovector encoding a cytokine of Table 50, or a functional variant thereof, is used for the treatment of a disease or disorder described herein.

In some embodiments, an effector described herein comprises an antibody molecule (e.g., an scFv) that binds a cytokine of Table 50. In some embodiments, an effector described herein comprises an antibody molecule (e.g., an scFv) that binds a cytokine receptor of Table 50. In some embodiments, the antibody molecule comprises a signal sequence.

Exemplary cytokines and cytokine receptors are described, e.g., in Akdis et al., “Interleukins (from IL-1 to IL-38), interferons, transforming growth factor (3, and TNF-α: Receptors, functions, and roles in diseases” October 2016 Volume 138, Issue 4, Pages 984-1010, which is herein incorporated by reference in its entirety, including Table I therein.

TABLE 51
Exemplary polypeptide hormones and receptors

| | | Entrez | |
|---|---|---|---|
| Hormone | Receptor | Gene ID | UniProt ID |
| Natriuretic Peptide, e.g., Atrial | NPRA, NPRB. NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | GHR | 2690 | P10912 |
| Human growth hormone (hGH) | hGH receptor (human GHR) | 2690 | P10912 |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (TH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 51, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 51 or functional variant thereof comprises a signal sequence, e.g., a signal sequence that is endogenous to the effector, or a heterologous signal sequence. In some embodiments, an anellovector encoding a hormone of Table 51, or a functional variant thereof, is used for the treatment of a disease or disorder described herein.

In some embodiments, an effector described herein comprises an antibody molecule (e.g., an scFv) that binds a hormone of Table 51. In some embodiments, an effector described herein comprises an antibody molecule (e.g., an scFv) that binds a hormone receptor of Table 51. In some embodiments, the antibody molecule comprises a signal sequence.

TABLE 52
Exemplary growth factors

| | | Entrez | |
|---|---|---|---|
| Growth Factor | | Gene ID | UniProt ID |
| PDGF family | | | |
| PDGF (e.g., PDGF-1, | PDGF receptor, e.g., | 5156 | P16234 |
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |
| VEGF family | | | |
| VEGF (e.g., isoforms | VEGFR-1, VEGFR-2 | 2321 | P17948 |
| VEGF 121, VEGF 165, | | | |
| VEGF 189, and VEGF | | | |
| 206) | | | |
| VEGF-B | VEGFR-1 | 2321 | P17949 |
| VEGF-C | VEGFR-2 and VEGFR-3 | 2324 | P35916 |
| P1GF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, | P11487, P08620, |
| FGF-7, FGF-8, FGF-9 | | 2254 | P12034, P10767, |
| | | | P21781, P55075, P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 52, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 52 or functional variant thereof comprises a signal sequence, e.g., a signal sequence that is endogenous to the effector, or a heterologous signal sequence. In some embodiments, an anellovector encoding a growth factor of Table 52, or a functional variant thereof, is used for the treatment of a disease or disorder described herein.

In some embodiments, an effector described herein comprises an antibody molecule (e.g., an scFv) that binds a growth factor of Table 52. In some embodiments, an effector described herein comprises an antibody molecule (e.g., an scFv) that binds a growth factor receptor of Table 52. In some embodiments, the antibody molecule comprises a signal sequence.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety.

TABLE 53
Clotting-associated factors

| | | Entrez | |
|---|---|---|---|
| Effector | Indication | Gene ID | UniProt ID |
| Factor I | Afibrinogenomia | 2243, 2266, | P02671, P02679, |
| (fibrinogen) | | 2244 | P02675 |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

In some embodiments, an effector described herein comprises a polypeptide of Table 53, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 53 or functional variant thereof comprises a signal sequence, e.g., a signal sequence that is endogenous to the effector, or a heterologous signal sequence. In some embodiments, an anellovector encoding a polypeptide of Table 53, or a functional variant thereof is used for the treatment of a disease or disorder of Table 53.

Exemplary Protein Replacement Therapeutics

Exemplary protein replacement therapeutics are described herein, e.g., in the tables below.

TABLE 54
Exemplary enzymatic effectors and corresponding indications

| | | Entrez | |
|---|---|---|---|
| Effector | deficiency | Gene ID | UniProt ID |
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenic Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, | Q8TDN7, |
| | (lipogranulomatosis) | 55331 | Q5QJU3, Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc:Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man:Man5GlcNAc2- | glycosylation CDG Id | | |
| PP-dolichol | | | |
| mannosyltransferase | | | |
| Dolichyl-P-glucose:Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose:Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, 8372, | Q12794, Q12891, |
| | IX (hyaluronidase deficiency) | 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyl- | | | |
| transferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl-1- | Mucolipidosis ML III | 79158 | Q3T906 |
| phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl-1- | Mucolipidosis ML III | 84572 | Q9UJJ9 |
| phosphotransferase, | (pseudo-Hurler's | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | Pl7405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc:dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-1-Iduronidase | Mucopolysaccharidosis MPS | 2517 | P04066 |
| | I H/S (Hurler-Scheie | | |
| | syndrome) | | |
| α-1-Iduronidase | Mucopolysaccharidosis MPS | 3425 | P35475 |
| | I-H (Hurler's syndrome) | | |
| α-1-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoffs disease | 3073 | P06865 |

In some embodiments, an effector described herein comprises an enzyme of Table 54, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, an anellovector encoding an enzyme of Table 54, or a functional variant thereof is used for the treatment of a disease or disorder of Table 54. In some embodiments, an anellovector is used to deliver uridine diphosphate glucuronyl-transferase or a functional variant thereof to a target cell, e.g., a liver cell. In some embodiments, an anellovector is used to deliver OCA1 or a functional variant thereof to a target cell, e.g., a retinal cell.

TABLE 55
Exemplary non-enzymatic effectors and corresponding indications

| | | Entrez | |
|---|---|---|---|
| Effector | Indication | Gene ID | UniProt ID |
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

In some embodiments, an effector described herein comprises an erythropoietin (EPO), e.g., a human erythropoietin (hEPO), or a functional variant thereof. In some embodiments, an anellovector encoding an erythropoietin, or a functional variant thereof is used for stimulating erythropoiesis. In some embodiments, an anellovector encoding an erythropoietin, or a functional variant thereof is used for the treatment of a disease or disorder, e.g., anemia. In some embodiments, an anellovector is used to deliver EPO or a functional variant thereof to a target cell, e.g., a red blood cell.

In some embodiments, an effector described herein comprises a polypeptide of Table 55, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, an anellovector encoding a polypeptide of Table 55, or a functional variant thereof is used for the treatment of a disease or disorder of Table 55. In some embodiments, an anellovector is used to deliver SMN or a functional variant thereof to a target cell, e.g., a cell of the spinal cord and/or a motor neuron. In some embodiments, an anellovector is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3; 26(10):2337-2356. doi: 10.1016/j.ymthe.2018.07.011. Epub 2018 Jul. 17.

In some embodiments, an effector described herein comprises a clotting factor, e.g., a clotting factor listed in Table 54 or Table 55 herein. In some embodiments, an effector described herein comprises a protein that, when mutated, causes a lysosomal storage disorder, e.g., a protein listed in Table 54 or Table 55 herein. In some embodiments, an effector described herein comprises a transporter protein, e.g., a transporter protein listed in Table 55 herein.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polyeptpide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| | | Protein |
|---|---|---|
| Target | Gene accession # | accession # |
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pmc/ |
| | | articles/PMC2732113/figure/F1/ |
| miR-222 | MI0000299 | |
| miR-302-367 | MIR302A And | https://www.ncbi.nlm.nih.gov/pmc/ |
| | MIR367 | articles/PMC4400607/ |

Transformation Factors

Therapeutic polypeptides described herein also include transformation factors, e.g., protein factors that transform fibroblasts into differentiated cell e.g., factors disclosed in Table 57 or functional variants thereof, e.g., a protein having at least 80%, 85%, 90%, 95%, 967%, 98%, 99% identity to a protein sequence disclosed in Table 57 by reference to its UniProt ID.

TABLE 57
Exemplary transformation factors

| | | | Protein |
|---|---|---|---|
| Target | Indication | Gene accession # | accession # |
| MESP1 | Organ Repair by | Gene ID: 55897 | EAX02066 |
| | transforming fibroblasts | | |
| ETS2 | Organ Repair by | GeneID: 2114 | NP_005230 |
| | transforming fibroblasts | | |
| HAND2 | Organ Repair by | GeneID: 9464 | NP_068808 |
| | transforming fibroblasts | | |
| MYOCARDIN | Organ Repair by | GeneID: 93649 | NP_001139784 |
| | transforming fibroblasts | | |
| ESRRA | Organ Repair by | Gene ID: 2101 | AAH92470 |
| | transforming fibroblasts | | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | GeneID: 7040 | NP_000651.3 |
| | transforming fibroblasts | | |
| WNT | Organ Repair by | Gene ID: 7471 | NP_005421 |
| | transforming fibroblasts | | |
| JAK | Organ Repair by | Gene ID: 3716 | NP_001308784 |
| | transforming fibroblasts | | |
| NOTCH | Organ Repair by | GeneID: 4851 | XP_011517019 |
| | transforming fibroblasts | | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| | | Protein |
|---|---|---|
| Target | Gene accession # | accession # |
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or anellovector described herein includes a polypeptide linked to a ligand that is capable of targeting a specific location, tissue, or cell.

Gene Editing Components

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cash, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector. In some embodiments, e.g., wherein the genetic element is an mRNA, a promoter may be absent from the genetic element. In some embodiments, a genetic element construct comprises a promoter that is used to drive production of the RNA genetic element.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, e.g., provided either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element construct may include an enhancer, e.g., a DNA sequence that is located adjacent to the DNA sequence that encodes a gene Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element may comprise one or more sequences or a fragment of a sequence from a substantially non-pathogenic virus having at least about 60%, 70% 80%, 85%, 90% 95%, 96%, 97%, 98% and 99% nucleotide sequence identity to any one of the nucleotide sequences described herein, e.g., Table 41.

TABLE 41
Examples of Anelloviruses and their sequences. Accessions numbers
and related sequence information may be obtained at www.ncbi.nlm.nih.gov/genbank/,
as referenced on Dec. 11, 2018.

| Accession # | Description |
|---|---|
| AB017613.1 | Torque teno virus 16 DNA, complete genome, isolate: TUS01 |
| AB026345.1 | TT virus genes for ORF1 and ORF2, complete cds, isolate: TRM1 |
| AB026346.1 | TT virus genes for ORF1 and ORF2, complete cds, isolate: TK16 |
| AB026347.1 | TT virus genes for ORF1 and ORF2, complete cds, isolate: TP1-3 |
| AB028669.1 | TT virus gene for ORF1 and ORF2, complete genome, isolate: TJN02 |
| AB030487.1 | TT virus gene for pORF2a, pORF2b, pOrf1, complete cds, clone: JaCHCTC19 |
| AB030488.1 | TT virus gene for pORF2a, pORF2b, pOrf1, complete cds, clone: JaBD89 |
| AB030489.1 | TT virus gene for pORF2a, pORF2b, pOrf1, complete cds, clone: JaBD98 |
| AB038340.1 | TT virus genes for ORF2s, ORF1, ORF3, complete cds |
| AB038622.1 | TT virus genes for ORF2, ORF1, ORF3, complete cds, isolate: TTVyon-LC011 |
| AB038623.1 | TT virus genes for ORF2, ORF1, ORF3, complete cds, isolate: TTVyon-KC186 |
| AB038624.1 | TT virus genes for ORF2, ORF1, ORF3, complete cds, isolate: TTVyon-KC197 |
| AB041821.1 | TT virus mRNA for VP1, complete cds |
| AB050448.1 | Torque teno virus genes for ORF1, ORF2, ORF3, ORF4, complete cds, isolate: |
| | TYM9 |
| AB060592.1 | Torque teno virus gene for ORF1, ORF2, ORF3, ORF4, clone: SAa-39 |
| AB060593.1 | Torque teno virus gene for ORF1, ORF2, ORF3, ORF4, complete cds, clone: |
| | SAa-38 |
| AB060595.1 | TT virus gene for ORF1, ORF2, ORF3, ORF4, complete cds, clone:SAj-30 |
| AB060596.1 | TT virus gene for ORF1, ORF2, ORF3, ORF4, complete cds, clone:SAf-09 |
| AB064596.1 | Torque teno virus DNA, complete genome, isolate: CT25F |
| AB064597.1 | Torque teno virus DNA, complete genome, isolate: CT30F |
| AB064599.1 | Torque teno virus DNA, complete genome, isolate: JT03F |
| AB064600.1 | Torque teno virus DNA, complete genome, isolate: JT05F |
| AB064601.1 | Torque teno virus DNA, complete genome, isolate: JT14F |
| AB064602.1 | Torque teno virus DNA, complete genome, isolate: JT19F |
| AB064603.1 | Torque teno virus DNA, complete genome, isolate: JT41F |
| AB064604.1 | Torque teno virus DNA, complete genome, isolate: CT39F |
| AB064606.1 | Torque teno virus DNA, complete genome, isolate: JT33F |
| AB290918.1 | Torque teno midi virus 1 DNA, complete genome, isolate: MD1-073 |
| AF079173.1 | TT virus strain TTVCHN1, complete genome |
| AF116842.1 | TT virus strain BDH1, complete genome |
| AF122914.3 | TT virus isolate JA20, complete genome |
| AF122917.1 | TT virus isolate JA4, complete genome |
| AF122919.1 | TT virus isolate JA10 unknown genes |
| AF129887.1 | TT virus TTVCHN2, complete genome |
| AF247137.1 | TT virus isolate TUPB, complete genome |
| AF254410.1 | TT virus ORF2 protein and ORF1 protein genes, complete cds |
| AF298585.1 | TT virus Polish isolate P/1C1, complete genome |
| AF315076.1 | TTV-like virus DXL1 unknown genes |
| AF315077.1 | TTV-like virus DXL2 unknown genes |
| AF345521.1 | TT virus isolate TCHN-G1 Orf2 and Orf1 genes, complete cds |
| AF345522.1 | TT virus isolate TCHN-E Orf2 and Orf1 genes, complete cds |
| AF345525.1 | TT virus isolate TCHN-D2 Orf2 and Orf1 genes, complete cds |
| AF345527.1 | TT virus isolate TCHN-C2 Orf2 and Orf1 genes, complete cds |
| AF345528.1 | TT virus isolate TCHN-F Orf2 and Orf1 genes, complete cds |
| AF345529.1 | TT virus isolate TCHN-G2 Orf2 and Orf1 genes, complete cds |
| AF371370.1 | TT virus ORF1, ORF3, and ORF2 genes, complete cds |
| AJ620212.1 | Torque teno virus, isolate tth6, complete genome |
| AJ620213.1 | Torque teno virus, isolate tth10, complete genome |
| AJ620214.1 | Torque teno virus, isolate tth11g2, complete genome |
| AJ620215.1 | Torque teno virus, isolate tth18, complete genome |
| AJ620216.1 | Torque teno virus, isolate tth20, complete genome |
| AJ620217.1 | Torque teno virus, isolate tth21, complete genome |
| AJ620218.1 | Torque teno virus, isolate tth3, complete genome |
| AJ620219.1 | Torque teno virus, isolate tth9, complete genome |
| AJ620220.1 | Torque teno virus, isolate tth16, complete genome |
| AJ620221.1 | Torque teno virus, isolate tth17, complete genome |
| AJ620222.1 | Torque teno virus, isolate tth25, complete genome |
| AJ620223.1 | Torque teno virus, isolate tth26, complete genome |
| AJ620224.1 | Torque teno virus, isolate tth27, complete genome |
| AJ620225.1 | Torque teno virus, isolate tth31, complete genome |
| AJ620226.1 | Torque teno virus, isolate tth4, complete genome |
| AJ620227.1 | Torque teno virus, isolate tth5, complete genome |
| AJ620228.1 | Torque teno virus, isolate tth14, complete genome |
| AJ620229.1 | Torque teno virus, isolate tth29, complete genome |
| AJ620230.1 | Torque teno virus, isolate tth7, complete genome |
| AJ620231.1 | Torque teno virus, isolate tth8, complete genome |
| AJ620232.1 | Torque teno virus, isolate tth13, complete genome |
| AJ620233.1 | Torque teno virus, isolate tth19, complete genome |
| AJ620234.1 | Torque teno virus, isolate tth22g4, complete genome |
| AJ620235.1 | Torque teno virus, isolate tth23, complete genome |
| AM711976.1 | TT virus sle1957 complete genome |
| AM712003.1 | TT virus sle1931 complete genome |
| AM712004.1 | TT virus sle1932 complete genome |
| AM712030.1 | TT virus sle2057 complete genome |
| AM712031.1 | TT virus sle2058 complete genome |
| AM712032.1 | TT virus sle2072 complete genome |
| AM712033.1 | TT virus sle2061 complete genome |
| AM712034.1 | TT virus sle2065 complete genome |
| AY026465.1 | TT virus isolate L01 ORF2 and ORF1 genes, complete cds |
| AY026466.1 | TT virus isolate L02 ORF2 and ORF1 genes, complete cds |
| DQ003341.1 | Torque teno virus clone P2-9-02 ORF2 (ORF2), ORF1A (ORF1A), and ORF1B |
| | (ORF1B) genes, complete cds |
| DQ003342.1 | Torque teno virus clone P2-9-07 ORF2 (ORF2), ORF1A (ORF1A), and ORF1B |
| | (ORF1B) genes, complete cds |
| DQ003343.1 | Torque teno virus clone P2-9-08 ORF2 (ORF2), ORF1A (ORF1A), and ORF1B |
| | (ORF1B) genes, complete cds |
| DQ003344.1 | Torque teno virus clone P2-9-16 ORF2 (ORF2), ORF1A (ORF1A), and ORF1B |
| | (ORF1B) genes, complete cds |
| DQ186994.1 | Torque teno virus clone P601 ORF2 (ORF2) and ORF1 (ORF1) genes, complete |
| | cds |
| DQ186995.1 | Torque teno virus clone P605 ORF2 (ORF2) and ORF1 (ORF1) genes, complete |
| | cds |
| DQ186996.1 | Torque teno virus clone BM1A-02 ORF2 (ORF2) and ORF1 (ORF1) genes, |
| | complete cds |
| DQ186997.1 | Torque teno virus clone BM1A-09 ORF2 (ORF2) and ORF1 (ORF1) genes, |
| | complete cds |
| DQ186998.1 | Torque teno virus clone BM1A-13 ORF2 (ORF2) and ORF1 (ORF1) genes, |
| | complete cds |
| DQ186999.1 | Torque teno virus clone BM1B-05 ORF2 (ORF2) and ORF1 (ORF1) genes, |
| | complete cds |
| DQ187000.1 | Torque teno virus clone BM1B-07 ORF2 (ORF2) and ORF1 (ORF1) genes, |
| | complete cds |
| DQ187001.1 | Torque teno virus clone BM1B-11 ORF2 (ORF2) and ORF1 (ORF1) genes, |
| | complete cds |
| DQ187002.1 | Torque teno virus clone BM1B-14 ORF2 (ORF2) and ORF1 (ORF1) genes, |
| | complete cds |
| DQ187003.1 | Torque teno virus clone BM1B-08 ORF2 (ORF2) gene, complete cds; and |
| | nonfunctional ORF1 (ORF1) gene, complete sequence |
| DQ187004.1 | Torque teno virus clone BM1C-16 ORF2 (ORF2) and ORF1 (ORF1) genes, |
| | complete cds |
| DQ187005.1 | Torque teno virus clone BM1C-10 ORF2 (ORF2) and ORF1 (ORF1) genes, |
| | complete cds |
| DQ187007.1 | Torque teno virus clone BM2C-25 ORF2 (ORF2) gene, complete cds; and |
| | nonfunctional ORF1 (ORF1) gene, complete sequence |
| DQ361268.1 | Torque teno virus isolate ViPi04 ORF1 gene, complete cds |
| EF538879.1 | Torque teno virus isolate CSC5 ORF2 and ORF1 genes, complete cds |
| EU305675.1 | Torque teno virus isolate LTT7 ORF1 gene, complete cds |
| EU305676.1 | Torque teno virus isolate LTT10 ORF1 gene, complete cds |
| EU889253.1 | Torque teno virus isolate ViPi08 nonfunctional ORF1 gene, complete sequence |
| FJ392105.1 | Torque teno virus isolate TW53A25 ORF2 gene, partial cds; and ORF1 gene, |
| | complete cds |
| FJ392107.1 | Torque teno virus isolate TW53A27 ORF2 gene, partial cds; and ORF1 gene, |
| | complete cds |
| FJ392108.1 | Torque teno virus isolate TW53A29 ORF2 gene, partial cds; and ORF1 gene, |
| | complete cds |
| FJ392111.1 | Torque teno virus isolate TW53A35 ORF2 gene, partial cds; and ORF1 gene, |
| | complete cds |
| FJ392112.1 | Torque teno virus isolate TW53A39 ORF2 gene, partial cds; and ORF1 gene, |
| | complete cds |
| FJ392113.1 | Torque teno virus isolate TW53A26 ORF2 gene, complete cds; and nonfunctional |
| | ORF1 gene, complete sequence |
| FJ392114.1 | Torque teno virus isolate TW53A30 ORF2 and ORF1 genes, complete cds |
| FJ392115.1 | Torque teno virus isolate TW53A31 ORF2 and ORF1 genes, complete cds |
| FJ392117.1 | Torque teno virus isolate TW53A37 ORF1 gene, complete cds |
| FJ426280.1 | Torque teno virus strain SIA109, complete genome |
| FR751500.1 | Torque teno virus complete genome, isolate TTV-HD23a (rheu215) |
| GU797360.1 | Torque teno virus clone 8-17, complete genome |
| HC742700.1 | Sequence 7 from Patent WO2010044889 |
| HC742710.1 | Sequence 17 from Patent WO2010044889 |
| JX134044.1 | TTV-like mini virus isolate TTMV LY1, complete genome |
| JX134045.1 | TTV-like mini virus isolate TTMV LY2, complete genome |
| KU243129.1 | TTV-like mini virus isolate TTMV-204, complete genome |
| KY856742.1 | TTV-like mini virus isolate zhenjiang, complete genome |
| LC381845.1 | Torque teno virus Human/Japan/KS025/2016 DNA, complete genome |
| MH648892.1 | Anelloviridae sp. isolate ctdc048, complete genome |
| MH648893.1 | Anelloviridae sp. isolate ctdh007, complete genome |
| MH648897.1 | Anelloviridae sp. isolate ctcb038, complete genome |
| MH648900.1 | Anelloviridae sp. isolate ctfc019, complete genome |
| MH648901.1 | Anelloviridae sp. isolate ctbb022, complete genome |
| MH648907.1 | Anelloviridae sp. isolate ctcf040, complete genome |
| MH648911.1 | Anelloviridae sp. isolate cthi018, complete genome |
| MH648912.1 | Anelloviridae sp. isolate ctea38, complete genome |
| MH648913.1 | Anelloviridae sp. isolate ctbg006, complete genome |
| MH648916.1 | Anelloviridae sp. isolate ctbg020, complete genome |
| MH648925.1 | Anelloviridae sp. isolate ctci019, complete genome |
| MH648932.1 | Anelloviridae sp. isolate ctid031, complete genome |
| MH648946.1 | Anelloviridae sp. isolate ctdb017, complete genome |
| MH648957.1 | Anelloviridae sp. isolate ctch017, complete genome |
| MH648958.1 | Anelloviridae sp. isolate ctbh011, complete genome |
| MH648959.1 | Anelloviridae sp. isolate ctbc020, complete genome |
| MH648962.1 | Anelloviridae sp. isolate ctif015, complete genome |
| MH648966.1 | Anelloviridae sp. isolate ctei055, complete genome |
| MH648969.1 | Anelloviridae sp. isolate ctjg000, complete genome |
| MH648976.1 | Anelloviridae sp. isolate ctcj064, complete genome |
| MH648977.1 | Anelloviridae sp. isolate ctbj022, complete genome |
| MH648982.1 | Anelloviridae sp. isolate ctbf014, complete genome |
| MH648983.1 | Anelloviridae sp. isolate ctbd027, complete genome |
| MH648985.1 | Anelloviridae sp. isolate ctch016, complete genome |
| MH648986.1 | Anelloviridae sp. isolate ctbd020, complete genome |
| MH648989.1 | Anelloviridae sp. isolate ctga035, complete genome |
| MH648990.1 | Anelloviridae sp. isolate cthf001, complete genome |
| MH648995.1 | Anelloviridae sp. isolate ctbd067, complete genome |
| MH648997.1 | Anelloviridae sp. isolate ctce026, complete genome |
| MH648999.1 | Anelloviridae sp. isolate ctfb058, complete genome |
| MH649002.1 | Anelloviridae sp. isolate ctjj046, complete genome |
| MH649006.1 | Anelloviridae sp. isolate ctcf030, complete genome |
| MH649008.1 | Anelloviridae sp. isolate ctbg025, complete genome |
| MH649011.1 | Anelloviridae sp. isolate ctbh052, complete genome |
| MH649014.1 | Anelloviridae sp. isolate ctba003, complete genome |
| MH649017.1 | Anelloviridae sp. isolate ctbb016, complete genome |
| MH649022.1 | Anelloviridae sp. isolate ctch023, complete genome |
| MH649023.1 | Anelloviridae sp. isolate ctbd051, complete genome |
| MH649028.1 | Anelloviridae sp. isolate ctbf9, complete genome |
| MH649038.1 | Anelloviridae sp. isolate ctbi030, complete genome |
| MH649039.1 | Anelloviridae sp. isolate ctca057, complete genome |
| MH649040.1 | Anelloviridae sp. isolate ctch033, complete genome |
| MH649042.1 | Anelloviridae sp. isolate ctjd005, complete genome |
| MH649045.1 | Anelloviridae sp. isolate ctdc021, complete genome |
| MH649051.1 | Anelloviridae sp. isolate ctdg044, complete genome |
| MH649056.1 | Anelloviridae sp. isolate ctcc062, complete genome |
| MH649061.1 | Anelloviridae sp. isolate ctid009, complete genome |
| MH649062.1 | Anelloviridae sp. isolate ctdc018, complete genome |
| MH649063.1 | Anelloviridae sp. isolate ctbf012, complete genome |
| MH649068.1 | Anelloviridae sp. isolate ctcc066, complete genome |
| MH649070.1 | Anelloviridae sp. isolate ctda011, complete genome |
| MH649077.1 | Anelloviridae sp. isolate ctbh034, complete genome |
| MH649083.1 | Anelloviridae sp. isolate ctdg028, complete genome |
| MH649084.1 | Anelloviridae sp. isolate ctii061, complete genome |
| MH649085.1 | Anelloviridae sp. isolate cteh021, complete genome |
| MH649092.1 | Anelloviridae sp. isolate ctbg012, complete genome |
| MH649101.1 | Anelloviridae sp. isolate ctif053, complete genome |
| MH649104.1 | Anelloviridae sp. isolate ctei657, complete genome |
| MH649106.1 | Anelloviridae sp. isolate ctca015, complete genome |
| MH649114.1 | Anelloviridae sp. isolate ctbf050, complete genome |
| MH649122.1 | Anelloviridae sp. isolate ctdc002, complete genome |
| MH649125.1 | Anelloviridae sp. isolate ctbb15, complete genome |
| MH649127.1 | Anelloviridae sp. isolate ctba013, complete genome |
| MH649137.1 | Anelloviridae sp. isolate ctbb000, complete genome |
| MH649141.1 | Anelloviridae sp. isolate ctbc019, complete genome |
| MH649142.1 | Anelloviridae sp. isolate ctid026, complete genome |
| MH649144.1 | Anelloviridae sp. isolate ctfj004. complete genome |
| MH649152.1 | Anelloviridae sp. isolate ctcj13, complete genome |
| MH649156.1 | Anelloviridae sp. isolate ctci006, complete genome |
| MH649157.1 | Anelloviridae sp. isolate ctbd025, complete genome |
| MH649158.1 | Anelloviridae sp. isolate ctbf005, complete genome |
| MH649161.1 | Anelloviridae sp. isolate ctcf045, complete genome |
| MH649165.1 | Anelloviridae sp. isolate ctcc29, complete genome |
| MH649169.1 | Anelloviridae sp. isolate ctib021, complete genome |
| MH649172.1 | Anelloviridae sp. isolate ctbh857, complete genome |
| MH649174.1 | Anelloviridae sp. isolate ctbj049, complete genome |
| MH649178.1 | Anelloviridae sp. isolate ctfc006, complete genome |
| MH649179.1 | Anelloviridae sp. isolate ctbe000, complete genome |
| MH649183.1 | Anelloviridae sp. isolate ctbb031, complete genome |
| MH649186.1 | Anelloviridae sp. isolate ctcb33, complete genome |
| MH649189.1 | Anelloviridae sp. isolate ctcc12, complete genome |
| MH649196.1 | Anelloviridae sp. isolate ctci060, complete genome |
| MH649199.1 | Anelloviridae sp. isolate ctbb017, complete genome |
| MH649203.1 | Anelloviridae sp. isolate cthc018, complete genome |
| MH649204.1 | Anelloviridae sp. isolate ctbj003, complete genome |
| MH649206.1 | Anelloviridae sp. isolate ctbg010, complete genome |
| MH649208.1 | Anelloviridae sp. isolate ctid008, complete genome |
| MH649209.1 | Anelloviridae sp. isolate ctbg056, complete genome |
| MH649210.1 | Anelloviridae sp. isolate ctda001, complete genome |
| MH649212.1 | Anelloviridae sp. isolate ctcf004, complete genome |
| MH649217.1 | Anelloviridae sp. isolate ctbe029, complete genome |
| MH649223.1 | Anelloviridae sp. isolate ctci016, complete genome |
| MH649224.1 | Anelloviridae sp. isolate ctce11, complete genome |
| MH649228.1 | Anelloviridae sp. isolate ctcf013, complete genome |
| MH649229.1 | Anelloviridae sp. isolate ctcb036, complete genome |
| MH649241.1 | Anelloviridae sp. isolate ctda027, complete genome |
| MH649242.1 | Anelloviridae sp. isolate ctbf003, complete genome |
| MH649254.1 | Anelloviridae sp. isolate ctjb007, complete genome |
| MH649255.1 | Anelloviridae sp. isolate ctbb023, complete genome |
| MH649256.1 | Anelloviridae sp. isolate ctca002, complete genome |
| MH649258.1 | Anelloviridae sp. isolate ctcg010, complete genome |
| MH649263.1 | Anelloviridae sp. isolate ctgh3, complete genome |
| MK012439.1 | Anelloviridae sp. isolate cthe000, complete genome |
| MK012440.1 | Anelloviridae sp. isolate ctjd008, complete genome |
| MK012448.1 | Anelloviridae sp. isolate ctch012, complete genome |
| MK012457.1 | Anelloviridae sp. isolate ctda009, complete genome |
| MK012458.1 | Anelloviridae sp. isolate ctcd015, complete genome |
| MK012485.1 | Anelloviridae sp. isolate ctfd011, complete genome |
| MK012489.1 | Anelloviridae sp. isolate ctba003, complete genome |
| MK012492.1 | Anelloviridae sp. isolate ctbb005, complete genome |
| MK012493.1 | Anelloviridae sp. isolate ctcj014, complete genome |
| MK012500.1 | Anelloviridae sp. isolate ctcb001, complete genome |
| MK012504.1 | Anelloviridae sp. isolate ctcj010, complete genome |
| MK012516.1 | Anelloviridae sp. isolate ctcf003, complete genome |
| NC_038336.1 | Torque teno virus 5 isolate TCHN-C1 Orf2 and Orf1 genes, complete cds |
| NC_038338.1 | Torque teno virus 11 isolate TCHN-D1 Orf2 and Orf1 genes, complete cds |
| NC_038339.1 | Torque teno virus 13 isolate TCHN-A Orf2 and Orf1 genes, complete cds |
| NC_038340.1 | Torque teno virus 20 ORF4, ORF3, ORF2, ORF1 genes, complete cds, clone: |
| | SAa-10 |
| NC_038341.1 | Torque teno virus 21 isolate TCHN-B ORF2 and ORF1 genes, complete cds |
| NC_038342.1 | Torque teno virus 23 ORF2, ORF1 genes, complete cds, isolate: s-TTV CH65-2 |
| NC_038343.1 | Torque teno virus 24 ORF4, ORF3, ORF2, ORF1 genes, complete cds, clone: |
| | SAa-01 |
| NC_038344.1 | Torque teno virus 29 ORF2, ORF1, ORF3 genes, complete cds, isolate: TTVyon- |
| | KC009 |
| NC_038345.1 | Torque teno mini virus 10 isolate LIL-y1 ORF2, ORF1, ORF3, and ORF4 genes, |
| | complete cds |
| NC_038346.1 | Torque teno mini virus 11 isolate LIL-y2 ORF2, ORF1, and ORF3 genes, |
| | complete cds |
| NC_038347.1 | Torque teno mini virus 12 isolate LIL-y3 ORF2, ORF1, ORF3, and ORF4 genes, |
| | complete cds |
| NC_038350.1 | Torque teno midi virus 3 isolate 2PoSMA ORF2 and ORF1 genes, complete cds |
| NC_038351.1 | Torque teno midi virus 4 isolate 6PoSMA ORF2, ORF1, and ORF3 genes, |
| | complete cds |
| NC_038352.1 | Torque teno midi virus 5 DNA, complete genome, isolate: MDJHem2 |
| NC_038353.1 | Torque teno midi virus 6 DNA, complete genome, isolate: MDJHem3-1 |
| NC_038354.1 | Torque teno midi virus 7 DNA, complete genome, isolate: MDJHem3-2 |
| NC_038355.1 | Torque teno midi virus 8 DNA, complete genome, isolate: MDJN1 |
| NC_038356.1 | Torque teno midi virus 9 DNA, complete genome, isolate: MDJN2 |
| NC_038357.1 | Torque teno midi virus 10 DNA, complete genome, isolate: MDJN14 |
| NC_038358.1 | Torque teno midi virus 11 DNA, complete genome, isolate: MDJN47 |
| NC_038359.1 | Torque teno midi virus 12 DNA, complete genome, isolate: MDJN51 |
| NC_038360.1 | Torque teno midi virus 13 DNA, complete genome, isolate: MDJN69 |
| NC_038361.1 | Torque teno midi virus 14 DNA, complete genome, isolate: MDJN97 |
| NC_038362.1 | Torque teno midi virus 15 DNA, complete genome, isolate: Pt-TTMDV210 |

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more. Since the genetic code is degenerate, a homologous nucleotide sequence can include any number of silent base changes, i.e., nucleotide substitutions that nonetheless encode the same amino acid.

### Proteinaceous Exterior

In some embodiments, the anellovector, e.g., synthetic anellovector, comprises a proteinaceous exterior that encloses the genetic element. The proteinaceous exterior can comprise a substantially non-pathogenic exterior protein that fails to elicit an unwanted immune response in a mammal. The proteinaceous exterior of the anellovectors typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 molecule and/or capsid protein sequence, e.g., as described herein. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to an Anellovirus ORF1 nucleic acid, e.g., as described herein.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to an Anellovirus ORF1 molecule as described herein.

In some embodiments, the ranges of amino acids with less sequence identity may provide one or more of the properties described herein and differences in cell/tissue/species specificity (e.g. tropism).

In some embodiments, the anellovector lacks lipids in the proteinaceous exterior. In some embodiments, the anellovector lacks a lipid bilayer, e.g., a viral envelope. In some embodiments, the interior of the anellovector is entirely covered (e.g., 100% coverage) by a proteinaceous exterior. In some embodiments, the interior of the anellovector is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: an arginine-rich region, jelly-roll region, N22 domain, hypervariable region, and/or C-terminal domain, e.g., of an ORF1 molecule, e.g., as described herein. In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 nucleic acid, e.g., as described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### III. Methods of Use

The anellovectors and compositions comprising anellovectors described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The anellovectors may be administered alone or formulated as a pharmaceutical composition.

The anellovectors may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of an anellovector or composition comprising same, e.g., as described herein, may result in delivery of a genetic element comprised by the anellovector to a target cell, e.g., in a subject.

An anellovector or composition thereof described herein, e.g., comprising an effector (e.g., an endogenous or exogenous effector), may be used to deliver the effector to a cell, tissue, or subject. In some embodiments, the anellovector or composition thereof is used to deliver the effector to bone marrow, blood, heart, GI or skin. Delivery of an effector by administration of an anellovector composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, a anellovector or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the anellovector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the anellovector described herein, or a composition comprising the anellovector, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector modulates (e.g., increases or decreases) an activity or function in a cell with which the anellovector is contacted. In some embodiments, the anellovector modulates (e.g., increases or decreases) the level or activity of a molecule (e.g., a nucleic acid or a protein) in a cell with which the anellovector is contacted. In some embodiments, the anellovector decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more.

### IV. Administration/Delivery

The composition (e.g., a pharmaceutical composition comprising an anellovector as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector to a subject. The method includes administering a pharmaceutical composition comprising an anellovector as described herein to the subject. In some embodiments, the administered anellovector replicates in the subject (e.g., becomes a part of the virome of the subject).

The pharmaceutical composition may include wild-type or native viral elements and/or modified viral elements. The anellovector may include one or more Anellovirus sequences (e.g., nucleic acid sequences or nucleic acid sequences encoding amino acid sequences thereof) or a sequence with at least about 60%, 65%, 70%, 75%, 80%, 85%, 90% 95%, 96%, 97%, 98% and 99% nucleotide sequence identity thereto. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more Anellovirus sequences (e.g., an Anellovirus ORF1 nucleic acid sequence). The anellovector may comprise a nucleic acid molecule encoding an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule). The anellovector may comprise a polypeptide comprising an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule).

In some embodiments, the anellovector is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an anellovector described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector is sufficient to compete with chronic or acute viral infection. In certain embodiments, the anellovector may be administered prophylactically to protect from viral infections (e.g. a provirotic). In some embodiments, the anellovector is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more). In some embodiments, treatment, treating, and cognates thereof comprise medical management of a subject (e.g., by administering an anellovector, e.g., an anellovector made as described herein), e.g., with the intent to improve, ameliorate, stabilize, prevent or cure a disease, pathological condition, or disorder. In some embodiments, treatment comprises active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder), and/or supportive treatment (treatment employed to supplement another therapy).

All references and publications cited herein are hereby incorporated by reference. The following examples are provided to further illustrate some embodiments of the present invention, but are not intended to limit the scope of the invention; it will be understood by their exemplary nature that other procedures, methodologies, or techniques known to those skilled in the art may alternatively be used.

### Examples

Table of Contents

Example 1: In vitro assembly of anellovectors using ORF1 produced using baculovirus
Example 2: In vitro assembly of Ring2 ORF1-based anellovectors encapsidating mRNA
Example 3: In vitro assembly of an mRNA-encapsidating anellovector using a modified ORF1 protein
Example 4: In vitro assembly of an mRNA-encapsidating anellovector using a modified mRNA
Example 5: Structural analysis of anellovirus ORF1 capsid proteins

### Example 1: In Vitro Assembly of Anellovectors Using ORF1 Produced Using Baculovirus

In this example, baculovirus constructs suitable for expression of Anellovirus proteins (e.g., ORF1) were generated by in vitro assembly.

In a first example, DNA encoding Ring2 ORF1 fused to an N-terminal HIS₆-tag (HIS-ORF1) was codon optimized for insect expression and cloned into the baculovirus expression vector pFASTbac system according to manufacturer instructions (ThermoFisher Scientific). 10 liters of insect cell culture (Sf9) was infected with Ring2 HIS-ORF1 baculovirus and the cells were harvested 3-days post-infection by centrifugation. The cells were lysed and the lysis was purified using a chelating resin column using standard methods in the field. The elution fraction containing HIS-ORF1 was dialyzed and treated with DNAse to digest host cell DNA. The resulting material was purified again using a chelating resin column and fractions containing ORF1 were retained for nucleic acid encapsidation and viral vector purification. ORF1-containing fractions were also analyzed by negative staining electron microscopy.

In a second example, DNA encoding Ring10 ORF1 fused to an N-terminal HIS₆-tag (HIS-ORF1) was codon optimized for insect expression and cloned into the baculovirus expression vector pFASTbac system according to manufacturer instructions (ThermoFisher Scientific). Insect cells (Sf9) were infected with Ring HIS-ORF1 baculovirus and the cells were harvested 3-days post-infection by centrifugation. The cells were lysed and the protein was purified using a chelating resin affinity column (HisTrap, GE Healthcare) using standard methods in the field. The resulting material was purified again using a heparin affinity column (Heparin HiTrap, GE Healthcare) and fractions containing ORF1 were analyzed by negative staining electron microscopy.

In a third example, DNA encoding chicken anemia virus (CAV) capsid protein (Vp1) fused to an N-terminal HIS₆-Flag-tag (HIS-Flag-Vp1) and helper protein (Vp2) were codon optimized for mammalian expression and cloned into a mammalian expression vector using a CMV promoter. Mammalian cells (293expi) were transfected with CAV Vp1 and Vp2 expression vectors. The cells were harvested 3-days post-infection by centrifugation. The cells were lysed and the lysis was purified using a the chelation and heparin purification process. The elution fraction containing Chicken Anemia Virus (CAV) Vp1 were analyzed by negative staining electron microscopy.

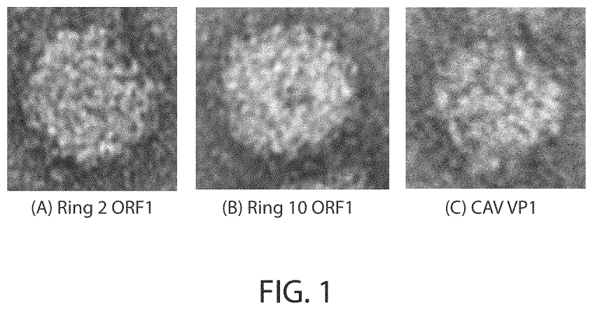

As shown in FIG. 1, both Ring 2 ORF1 and Ring 10 ORF1 showed a propensity to form ˜35 nm virus-like particles.

Nucleic acid encapsidation and viral vector purification: Ring ORF1 (wildtype protein, chimeric protein, or fragments thereof) will be treated with conditions sufficient to dissociate VLPs or viral capsids to enable reassembly with nucleic acid cargo. Nucleic acid cargo can be defined, for example, RNA which encodes a gene of interest that one wants to deliver as a therapeutic agent. Nucleic acid cargo of defined concentration will be combined with Ring ORF1 of defined concentration and treated with conditions sufficient to permit nucleic acid encapsidation and the resulting particle, defined as viral vector, will be subsequently purified using standard viral purification procedures.

### Example 2: In Vitro Assembly of Ring2 ORF1-Based Anellovectors Encapsidating mRNA

Ring2 ORF1 is purified by size exclusion chromatography (SEC) with mobile phases including Tris pH 8.0 with 500 mM NaCl, Tris pH 8.0 with 500 mM NaCl with 0.1% SDS, CAPS buffer pH 10.5 with 150 mM NaCl, CAPS buffer pH 10.5 with 500 mM NaCl or CAPS buffer pH 10.5 with 500 mM NaCl with 0.1% SDS to dissociate viral particles or VLPs into dispersed protein or capsomers.

In a first example, the ORF1 is mixed with mRNA, a fluorescently labeled mRNA or an mRNA transgene chemically conjugated to a segment of ssDNA shown in Example 1 to be competent for inducing vector formation. Viral vectors are formed through dialysis and SEC purification using Tris pH 8.0 buffer to isolate the anellovector encapsidating RNA (e.g., as measured by retained fluorescent absorption). Anellovector assembly is further evaluated by biophysical assessment such as DLS or electron microscopy.

In a second example, purified ORF1 is treated with 1 M NaCl with 0.1% SDS dissociate oligomers or VLPs into dispersed protein or capsomers. ORF1 is then mixed with mRNA, such as an mRNA that translate a gene of interest (e.g., a reporter gene, e.g., GFP, mCherry; or an effector of interest, e.g., EPO), and dialyzed against Tris pH 8.0 with 150 mM NaCl to permit VLP formation. The subsequent complex is purified by SEC using Tris pH 8.0 buffer to isolate the AV vector encapsidating mRNA. Anellovector vector assembly may be further evaluated by in vitro or in vivo readout, for example, by transducing cells and observing the expression of the reporter gene (e.g., mCherry or GFP) or through expression of an effector of interest (e.g., using an ELISA to detect the expression of a gene, such as EPO).

In Vitro Assembly of Ring2 ORF1-Based Anellovectors Encapsidating GFP mRNA

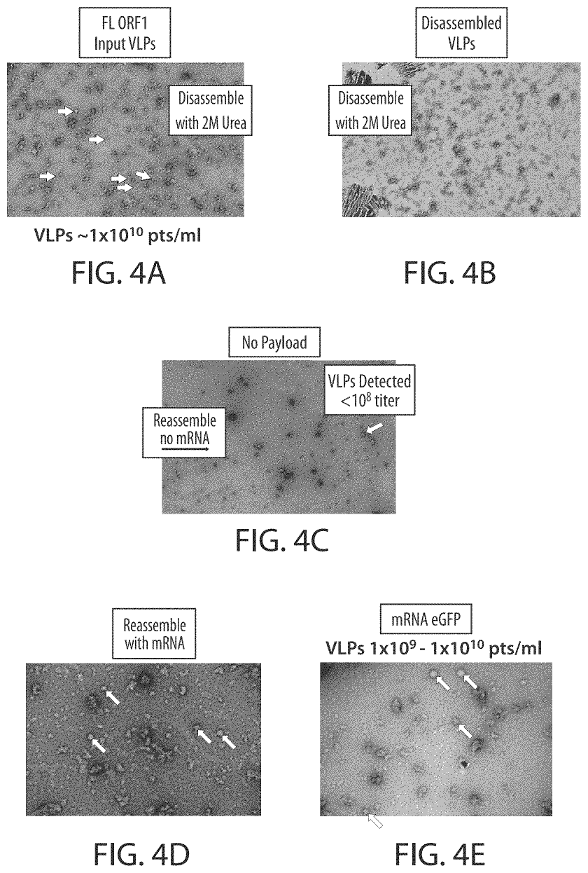

In a further example, Ring2 ORF1 protein was expressed as a full-length protein in insect cells and assembled VLPs were purified by a heparin affinity column followed by size exclusion chromatography (SEC) using a Tris buffer mobile phase. VLPs formed from the isolated Ring2 ORF1 proteins were observed with negative staining electron microscopy (EM) and had an estimated particle titer of 10¹⁰ particles/ml (pts/ml; FIG. 4A). The VLPs were treated with 2 molar (2M) urea to disassemble the VLPs. Reimaging by EM showed no VLPs observed (FIG. 4B). Urea-treated VLPs were then dialyzed to remove urea either in the absence of mRNA (FIG. 4C) or in the presence of ˜10× excess mRNA encoding GFP (FIGS. 4D and 4E). For VLP samples treated with urea and dialyzed in absence of mRNA, few particles (less than 10⁸ particles/ml; FIG. 4C) were observed by EM. In contrast, dialysis in the presence of excess mRNA resulted in the observation of substantially higher titers of particles (˜10⁹-10¹⁰ particles/ml; FIG. 4D and eE) by EM. These data demonstrate that disassembly and reassembly of VLPs is more efficient in the presence of mRNA, and indicate that Anellovirus ORF1 protein can be used to encapsidate mRNA in vitro to form anellovectors.

### Example 3: In Vitro Assembly of an mRNA-Encapsidating Anellovector Using a Modified ORF1 Protein

In this example, packaging of an mRNA genetic element is improved by modifying the ORF1 protein to harbor contact residues that bind mRNA. In this example, ssDNA contact residues and/or jellyroll beta strands that contact ssDNA and/or the N-terminal arginine-rich motif (ARM) can be replaced with components of an mRNA binding viral protein or other mRNA-binding protein to permit efficient binding and packaging of mRNA. This mRNA-binding chimeric ORF1 is then treated with 1 M NaCl with 0.1% SDS to dissociate oligomers or VLPs into dispersed protein or capsomers. The chimeric ORF1 is then mixed with mRNA, such as an mRNA that encodes a gene of interest (e.g., a reporter gene, e.g., GFP, mCherry; or an effector of interest, e.g., EPO), and dialyzed against Tris pH 8.0 with 150 mM NaCl to permit VLP formation. The subsequent complex is purified by SEC using Tris pH 8.0 buffer to isolate the anellovector encapsidating mRNA. Anellovector assembly can be further evaluated by in vitro or in vivo readout, for example, by transducing cells and observing the expression of the reporter gene (e.g., mCherry or GFP) or through expression of an effector of interest (such as using an ELISA to detect the expression of a gene such as EPO).

Exemplary modifications to ORF1 molecules: Ring ORF1 molecules that may be used in the methods described herein include, for example, several wildtype Anellovirus ORF1 proteins; CAV capsid protein (VP1) variants; Anellovirus ORF1 proteins harboring mutations to improve assembly efficiency, yield or stability; and chimeric ORF1 strains or functional fragments thereof. In some instances, affinity tags are attached to the ORF1 molecule, e.g., at the N-terminus (SEQ ID NOs: 561-562). In some instances, the ORF1 molecules are untagged proteins. Ring ORF1 molecules may be expressed alone or in combination with any number of helper proteins including, but not limited to, Anellovirus ORF2 and/or ORF3 proteins.

Ring ORF1 proteins harboring mutations to improve assembly efficiency may include, but are not limited to, ORF1 proteins that harbor mutations introduced into the N-terminal arginine-rich motif (ARM), for example, to alter the pI of the ARG arm, which may permit pH sensitive nucleic acid binding to trigger particle assembly (SEQ ID NOs: 563-565). ORF1 mutations that improve stability may include, for example, mutations to the interprotomer contacting beta strands F and G of the canonical jellyroll beta-barrel (F and G beta strands), e.g., to alter the hydrophobic state of the protomer surface and/or to make capsid formation more thermodynamically favored.

Chimeric ORF1 proteins may include, but are not limited to, ORF1 proteins which have a portion or portions of their sequence replaced with comparable portions from another capsid protein, such as BFDV capsid protein, Hepatitis E capsid protein (e.g., the ARG arm and/or F and G beta strands, or comparable components thereof). Chimeric ORF1 proteins may also include ORF1 proteins which have a portion or portions of their sequence replaced with comparable portions of another Anellovirus ORF1 protein (such as jelly roll fragments or the C-terminal portion of Ring 2 ORF1 replaced with comparable portions of Ring 9 ORF1; see, e.g., SEQ ID NOs: 568-575).

Generally, ORF1 molecules can be purified using purification techniques including, but not limited to, chelating purification, heparin purification, gradient sedimentation purification and/or SEC purification.

### Example 4: In Vitro Assembly of an mRNA-Encapsidating Anellovector Using a Modified mRNA

In this example, encapsidation of an mRNA-based genetic element is optimized by binding the mRNA molecule to ssDNA or by modifying the mRNA transgene in such a way that that a section of the backbone would permit binding to the ssDNA contact residues of wildtype ORF1. The mRNA generally encodes a gene of interest, such as a reporter gene (e.g., GFP or mCherry), and/or an effector gene (e.g., EPO).

In one example, modified ssDNA that can bind ORF1 by virtue of its sugar-chain backbone, but which can also pair with mRNA non-covalently, is mixed with an mRNA of interest to produce an mRNA/DNA complex. This mRNA/DNA complex can then be encapsidated using a Ring ORF1 to form an anellovector, for example, as described below.

In another example, an mRNA molecule is synthesized with a section or sections of the mRNA molecule harboring a DNA backbone permitting binding and encapsidation with ORF1, while retaining the portion of the mRNA that encodes a gene (e.g., a reporter gene or an effector gene) to be delivered. This mRNA/DNA hybrid molecule can then be encapsidated using a Ring ORF1 to form an anellovector, for example, as described below.

Encapsidation by in vitro assembly: The mRNA/DNA genetic elements described above are then encapsidated by in vitro assembly. Briefly, anellovector ORF1 is then treated with 1 M NaCl with 0.1% SDS to dissociate oligomers or VLPs into dispersed protein or capsomers. The ORF1 is then mixed with the synthetic mRNA complexes or hybrid molecules and dialyzed against Tris pH 8.0 with 150 mM NaCl to permit VLP formation. The subsequent particle is purified by SEC using Tris pH 8.0 buffer to isolate the anellovector encapsidating mRNA. Anellovector assembly could be further evaluated by in vitro or in vivo readout by transducing cells and observing the expression of the reporter gene or effector gene, e.g., as described herein.

### Example 5: Structural Analysis of Anellovirus ORF1 Capsid Proteins

Anelloviruses share predicted structural features to other well-characterized viruses such as the avian pathogens Beak and Feather Disease Virus (BFDV) or Chicken Anemia Virus (CAV).

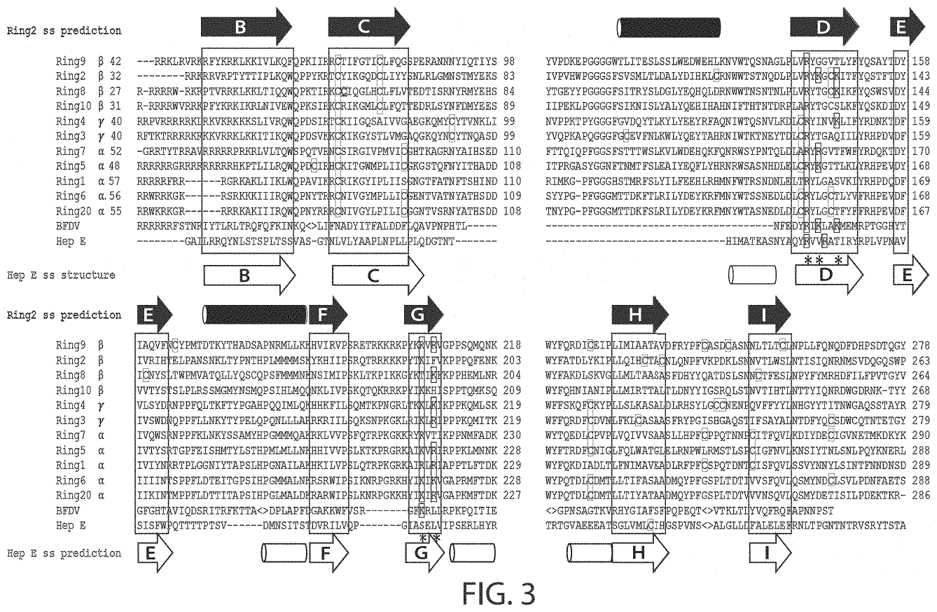

Anellovirus ORF1 capsid proteins contain an N-terminal ARM sequence similar to that of BFDV. Secondary structure prediction showed that the first ˜250 residues of ORF1, dependent on strain, includes 8 predicted beta strands (FIG. 3). When the 8 predicted beta strands of ORF1, named B through I following jelly roll domain naming conventions, are aligned guided by the secondary structure prediction to the capsid proteins of BFDV and Hep E, conserved lysine and arginine residues in ORF1 align with the known ssDNA contact residues of BFDV and Hep E capsid proteins (FIG. 3, denoted by asterisk).

### Sequences

The sequences listed below are annotated as follows. Bolded and underlined text indicates a sequence comprising a His6 tag (HHHHHH) used for chelating purification and a Flag tag (DYKDDDDK), a strong epitope used, e.g., for Western blot detection of low-expressing proteins. Bolded and italicized sequences indicate Ring9 ORF1 sequence or portions thereof. Unbolded, non-underlined sequences are Ring2 sequences or portions thereof. Unbolded, underlined sequences are from Beak and Feather Disease Virus (BFDV). Gray highlighting indicates the positions of lysine-to-histidine mutations, e.g., in the arginine-rich region and the first beta strand of Ring 9 ORF1.

SEQ ID NO: 561:
Ring 2 N-terminal HIS-FLAG-3CProtease-ORF1:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN
QDLPLVRYKGCKITFYQSTFTDYIVRIHTELPANSNKLTYPNTHPLMMMMSKYKHIIPSRQTRRKKKPYTKIFVKPPPQFE
NKWYFATDLYKIPLLQIHCTACNLQNPFVKPDKLSNNVTLWSLNTISIQNRNMSVDQGQSWPFKILGTQSFYFYFYTGA
NLPGDTTQIPVADLLPLTNPRINRPGQSLNEAKITDHITFTEYKNKFTNYWGNPFNKHIQEHLDMILYSLKSPEAIKNEWT
TENMKWNQLNNAGTMALTPFNEPIFTQIQYNPDRDTGEDTQLYLLSNATGTGWDPPGIPELILEGFPLWLIYWGFAD
FQKNLKKVTNIDTNYMLVAKTKFTQKPGTFYLVILNDTFVEGNSPYEKQPLPEDNIKWYPQVQYQLEAQNKLLQTGPFT
PNIQGQLSDNISMFYKFYFKWGGSPPKAINVENPAHQIQYPIPRNEHETTSLQSPGEAPESILYSFDYRHGNYTTTALSRI
SQDWALKDTVSKITEPDRQQLLKQALECLQISEETQEKKEKEVQQLISNLRQQQQLYRERIISLLKDQ
SEQ ID NO: 562:
Ring 9 N-terminal HIS-FLAG-3CProtease-ORF1:

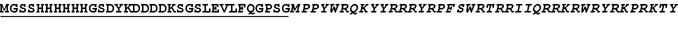

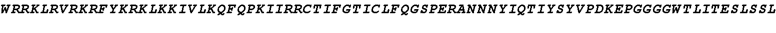

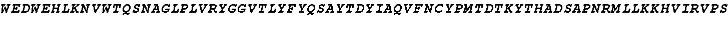

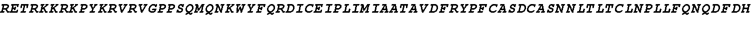

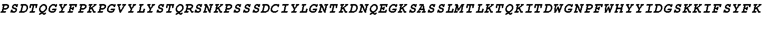

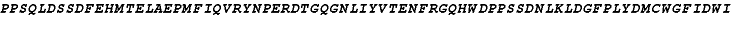

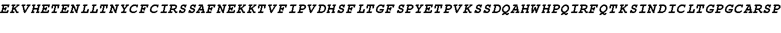

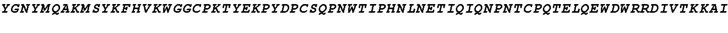

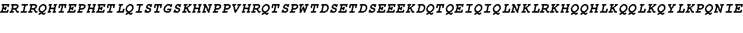

SEQ ID NO: 563:
Ring 2 ORF1 with ARG arm of Ring 9 (Ring 291)

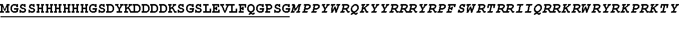

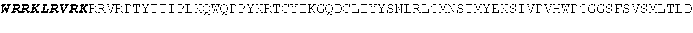

ALYDIHKLCRNWWTSTNQDLPLVRYKGCKITFYQSTFTDYIVRIHTELPANSNKLTYPNTHPLMMMMSKYKHIIPSRQT
RRKKKPYTKIFVKPPPQFENKWYFATDLYKIPLLQIHCTACNLQNPFVKPDKLSNNVTLWSLNTISIQNRNMSVDQGQS
WPFKILGTQSFYFYFYTGANLPGDTTQIPVADLLPLTNPRINRPGQSLNEAKITDHITFTEYKNKFTNYWGNPFNKHIQE
HLDMILYSLKSPEAIKNEWTTENMKWNQLNNAGTMALTPFNEPIFTQIQYNPDRDTGEDTQLYLLSNATGTGWDPPG
IPELILEGFPLWLIYWGFADFQKNLKKVTNIDTNYMLVAKTKFTQKPGTFYLVILNDTFVEGNSPYEKQPLPEDNIKWYP
QVQYQLEAQNKLLQTGPFTPNIQGQLSDNISMFYKFYFKWGGSPPKAINVENPAHQIQYPIPRNEHETTSLQSPGEAPE
SILYSFDYRHGNYTTTALSRISQDWALKDTVSKITEPDRQQLLKQALECLQISEETQEKKEKEVQQLISNLRQQQQLYRERI
ISLLKDQ
SEQ ID NO: 564:
Ring 2 ORF1 with ARG arm and Beta strands 1 + 2 722 epitope of Ring 9 (Ring 292)

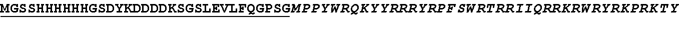

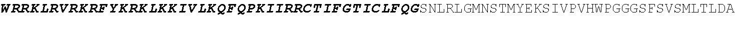

LYDIHKLCRNWWTSTNQDLPLVRYKGCKITFYQSTFTDYIVRIHTELPANSNKLTYPNTHPLMMMMSKYKHIIPSRQTR
RKKKPYTKIFVKPPPQFENKWYFATDLYKIPLLQIHCTACNLQNPFVKPDKLSNNVTLWSLNTISIQNRNMSVDQGQSW
PFKILGTQSFYFYFYTGANLPGDTTQIPVADLLPLTNPRINRPGQSLNEAKITDHITFTEYKNKFTNYWGNPFNKHIQEHL
DMILYSLKSPEAIKNEWTTENMKWNQLNNAGTMALTPFNEPIFTQIQYNPDRDTGEDTQLYLLSNATGTGWDPPGIP
ELILEGFPLWLIYWGFADFQKNLKKVTNIDTNYMLVAKTKFTQKPGTFYLVILNDTFVEGNSPYEKQPLPEDNIKWYPQV
QYQLEAQNKLLQTGPFTPNIQGQLSDNISMFYKFYFKWGGSPPKAINVENPAHQIQYPIPRNEHETTSLQSPGEAPESIL
YSFDYRHGNYTTTALSRISQDWALKDTVSKITEPDRQQLLKQALECLQISEETQEKKEKEVQQLISNLRQQQQLYRERIISL
LKDQ
SEQ ID NO: 565:
Ring 9 with LYS/HIS mutations in ARG arm and first beta strand

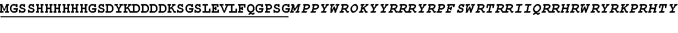

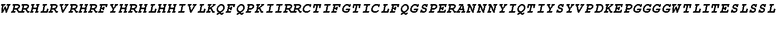

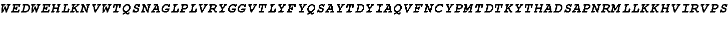

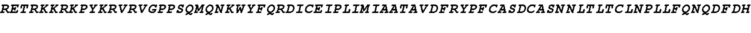

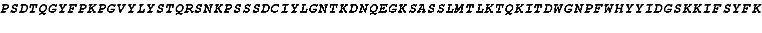

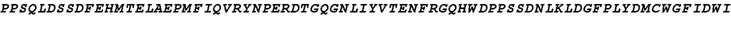

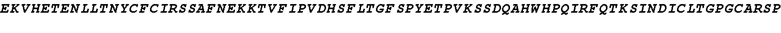

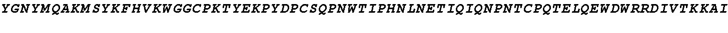

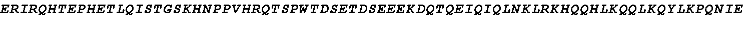

SEQ ID NO: 566:
Ring 9 with ARG arm of BFDV:

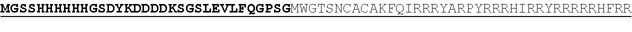

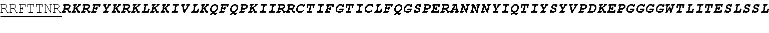

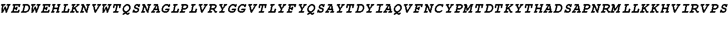

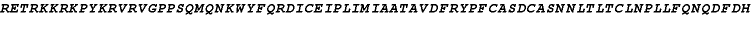

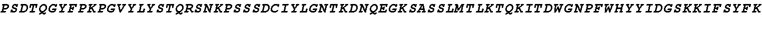

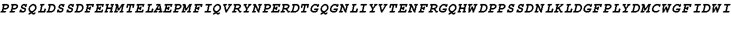

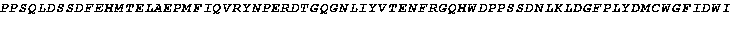

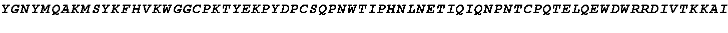

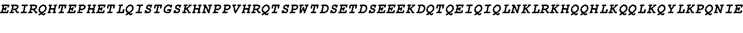

SEQ ID NO: 567:
Ring 9 with beta strands F and G of BFDV capsid protein:

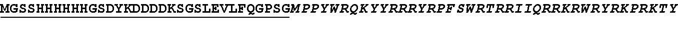

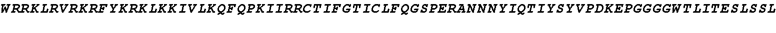

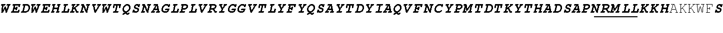

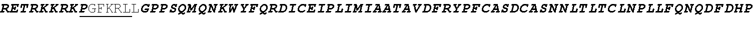

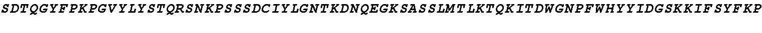

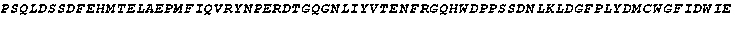

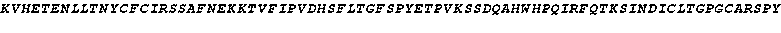

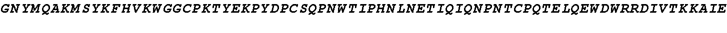

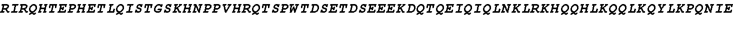

SEQ ID NO: 568:
Ring 2 with beta C of Ring 9:

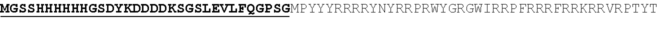

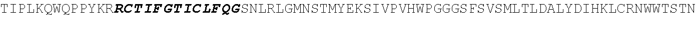

QDLPLVRYKGCKITFYQSTFTDYIVRIHTELPANSNKLTYPNTHPLMMMMSKYKHIIPSRQTRRKKKPYTKIFVKPPPQFE
NKWYFATDLYKIPLLQIHCTACNLQNPFVKPDKLSNNVTLWSLNTISIQNRNMSVDQGQSWPFKILGTQSFYFYFYTGA
NLPGDTTQIPVADLLPLTNPRINRPGQSLNEAKITDHITFTEYKNKFTNYWGNPFNKHIQEHLDMILYSLKSPEAIKNEWT
TENMKWNQLNNAGTMALTPFNEPIFTQIQYNPDRDTGEDTQLYLLSNATGTGWDPPGIPELILEGFPLWLIYWGFAD
FQKNLKKVTNIDTNYMLVAKTKFTQKPGTFYLVILNDTFVEGNSPYEKQPLPEDNIKWYPQVQYQLEAQNKLLQTGPFT
PNIQGQLSDNISMFYKFYFKWGGSPPKAINVENPAHQIQYPIPRNEHETTSLQSPGEAPESILYSFDYRHGNYTTTALSRI
SQDWALKDTVSKITEPDRQQLLKQALECLQISEETQEKKEKEVQQLISNLRQQQQLYRERIISLLKDQ
SEQ ID NO: 569:
Ring 2 with linker 1 of Ring 9:
MGSSHHHHHHGSDYKDDDDKSGSLEVLFQGPSGMPYYYRRRRYNYRRPRWYGRGWIRRPFRRRFRRKRRVRPTYT

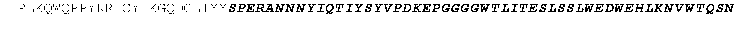

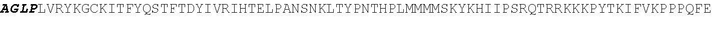

NKWYFATDLYKIPLLQIHCTACNLQNPFVKPDKLSNNVTLWSLNTISIQNRNMSVDQGQSWPFKILGTQSFYFYFYTGA
NLPGDTTQIPVADLLPLTNPRINRPGQSLNEAKITDHITFTEYKNKFTNYWGNPFNKHIQEHLDMILYSLKSPEAIKNEWT
TENMKWNQLNNAGTMALTPFNEPIFTQIQYNPDRDTGEDTQLYLLSNATGTGWDPPGIPELILEGFPLWLIYWGFAD
FQKNLKKVTNIDTNYMLVAKTKFTQKPGTFYLVILNDTFVEGNSPYEKQPLPEDNIKWYPQVQYQLEAQNKLLQTGPFT
PNIQGQLSDNISMFYKFYFKWGGSPPKAINVENPAHQIQYPIPRNEHETTSLQSPGEAPESILYSFDYRHGNYTTTALSRI
SQDWALKDTVSKITEPDRQQLLKQALECLQISEETQEKKEKEVQQLISNLRQQQQLYRERIISLLKDQ
SEQ ID NO: 570:
Ring 2 with beta strand D of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN

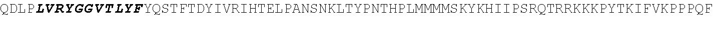

ENKWYFATDLYKIPLLQIHCTACNLQNPFVKPDKLSNNVTLWSLNTISIQNRNMSVDQGQSWPFKILGTQSFYFYFYTG
ANLPGDTTQIPVADLLPLTNPRINRPGQSLNEAKITDHITFTEYKNKFTNYWGNPFNKHIQEHLDMILYSLKSPEAIKNE
WTTENMKWNQLNNAGTMALTPFNEPIFTQIQYNPDRDTGEDTQLYLLSNATGTGWDPPGIPELILEGFPLWLIYWGF
ADFQKNLKKVTNIDTNYMLVAKTKFTQKPGTFYLVILNDTFVEGNSPYEKQPLPEDNIKWYPQVQYQLEAQNKLLQTG
PFTPNIQGQLSDNISMFYKFYFKWGGSPPKAINVENPAHQIQYPIPRNEHETTSLQSPGEAPESILYSFDYRHGNYTTTAL
SRISQDWALKDTVSKITEPDRQQLLKQALECLQISEETQEKKEKEVQQLISNLRQQQQLYRERIISLLKDQ
SEQ ID NO: 571:
Ring 2 with linker 2 of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN

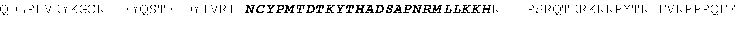

NKWYFATDLYKIPLLQIHCTACNLQNPFVKPDKLSNNVTLWSLNTISIQNRNMSVDQGQSWPFKILGTQSFYFYFYTGA
NLPGDTTQIPVADLLPLTNPRINRPGQSLNEAKITDHITFTEYKNKFTNYWGNPFNKHIQEHLDMILYSLKSPEAIKNEWT
TENMKWNQLNNAGTMALTPFNEPIFTQIQYNPDRDTGEDTQLYLLSNATGTGWDPPGIPELILEGFPLWLIYWGFAD
FQKNLKKVTNIDTNYMLVAKTKFTQKPGTFYLVILNDTFVEGNSPYEKQPLPEDNIKWYPQVQYQLEAQNKLLQTGPFT
PNIQGQLSDNISMFYKFYFKWGGSPPKAINVENPAHQIQYPIPRNEHETTSLQSPGEAPESILYSFDYRHGNYTTTALSRI
SQDWALKDTVSKITEPDRQQLLKQALECLQISEETQEKKEKEVQQLISNLRQQQQLYRERIISLLKDQ
SEQ ID NO: 572:
Ring 2 with beta strand G DNA binding of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN

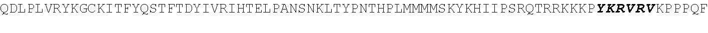

ENKWYFATDLYKIPLLQIHCTACNLQNPFVKPDKLSNNVTLWSLNTISIQNRNMSVDQGQSWPFKILGTQSFYFYFYTG
ANLPGDTTQIPVADLLPLTNPRINRPGQSLNEAKITDHITFTEYKNKFTNYWGNPFNKHIQEHLDMILYSLKSPEAIKNE
WTTENMKWNQLNNAGTMALTPFNEPIFTQIQYNPDRDTGEDTQLYLLSNATGTGWDPPGIPELILEGFPLWLIYWGF
ADFQKNLKKVTNIDTNYMLVAKTKFTQKPGTFYLVILNDTFVEGNSPYEKQPLPEDNIKWYPQVQYQLEAQNKLLQTG
PFTPNIQGQLSDNISMFYKFYFKWGGSPPKAINVENPAHQIQYPIPRNEHETTSLQSPGEAPESILYSFDYRHGNYTTTAL
SRISQDWALKDTVSKITEPDRQQLLKQALECLQISEETQEKKEKEVQQLISNLRQQQQLYRERIISLLKDQ
SEQ ID NO: 573:
Ring 2 with beta strand F interprotomer contact of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN

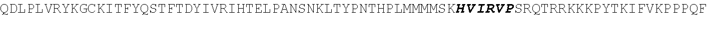

ENKWYFATDLYKIPLLQIHCTACNLQNPFVKPDKLSNNVTLWSLNTISIQNRNMSVDQGQSWPFKILGTQSFYFYFYTG
ANLPGDTTQIPVADLLPLTNPRINRPGQSLNEAKITDHITFTEYKNKFTNYWGNPFNKHIQEHLDMILYSLKSPEAIKNE
WTTENMKWNQLNNAGTMALTPFNEPIFTQIQYNPDRDTGEDTQLYLLSNATGTGWDPPGIPELILEGFPLWLIYWGF
ADFQKNLKKVTNIDTNYMLVAKTKFTQKPGTFYLVILNDTFVEGNSPYEKQPLPEDNIKWYPQVQYQLEAQNKLLQTG
PFTPNIQGQLSDNISMFYKFYFKWGGSPPKAINVENPAHQIQYPIPRNEHETTSLQSPGEAPESILYSFDYRHGNYTTTAL
SRISQDWALKDTVSKITEPDRQQLLKQALECLQISEETQEKKEKEVQQLISNLRQQQQLYRERIISLLKDQ
SEQ ID NO: 574:
Ring 2 ORF1 with strand H and I with C-terminal fragment of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN
QDLPLVRYKGCKITFYQSTFTDYIVRIHTELPANSNKLTYPNTHPLMMMMSKYKHIIPSRQTRRKKKPYTKIFVKPPPQFE

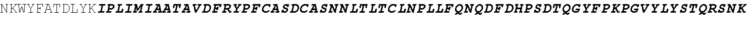

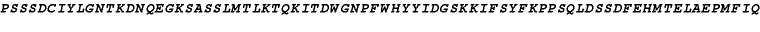

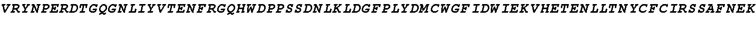

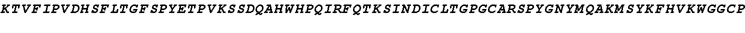

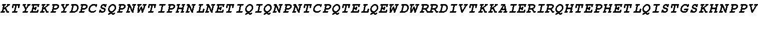

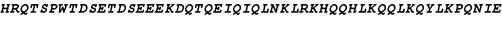

SEQ ID NO: 575:
Ring 2 ORF1 with strand I and C-terminal fragment of Ring 9:
MGSSHHHHHHGSDYKDDDDKSGSLEVLFQGPSGMPYYYRRRRYNYRRPRWYGRGWIRRPFRRRFRRKRRVRPTYT
TIPLKQWQPPYKRTCYIKGQDCLIYYSNLRLGMNSTMYEKSIVPVHWPGGGSFSVSMLTLDALYDIHKLCRNWWTSTN
QDLPLVRYKGCKITFYQSTFTDYIVRIHTELPANSNKLTYPNTHPLMMMMSKYKHIIPSRQTRRKKKPYTKIFVKPPPQFE

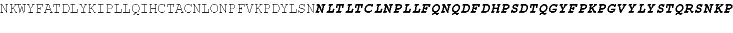

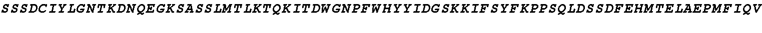

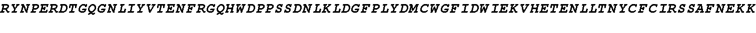

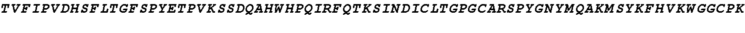

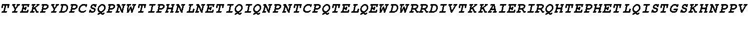

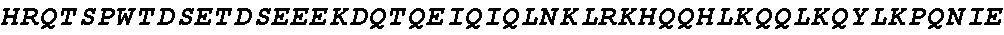

What is claimed is:

1. A particle comprising:
a) proteinaceous exterior comprising an ORF1 molecule; and
b) a genetic element comprising mRNA and encoding an exogenous effector,
wherein the genetic element is enclosed within the proteinaceous exterior.

2. The particle of claim 1, wherein the exogenous effector comprises a therapeutic polypeptide.

3. The particle of claim 1, wherein the exogenous effector comprises a human protein.

4. The particle of claim 1, wherein the exogenous effector comprises:
(i) a cytosolic polypeptide or cytosolic peptide;
(ii) a regulatory intracellular polypeptide;
(iii) a secreted polypeptide or peptide;
(iv) a protein replacement therapeutic;
(v) an enzyme;
(vi) a component of a gene editing system;
(vii) a membrane receptor; or
(viii) a membrane transporter.

5. The particle of claim 1, wherein the genetic element comprises a chemically modified ribonucleotide.

6. The particle of claim 1, wherein the genetic element consists of or consists essentially of RNA.

7. The particle of claim 1, wherein the genetic element is about 50-60 60-70, 70-80, 80-90, 90-100, 100-125, 125-150, 150-200, 200-300, 300-400, 400-500, 500-600, 600-700, 700-800, 800-900, 900-1000, 1000-1500, 1500-2000, 2000-2500, 2500-3000, 3000-3500, 3500-4000, or 4000-4500 nucleotides in length.

8. The particle of claim 1, wherein the sequence encoding the exogenous effector is at least about 30, 40, 50, 60, 70, 80, 90, 100, 125, 150, 175, 200, 250, 300, 400, 500, 600, 700, 800, 900, 1000, 1500, 2000, 2500, or 3000 nucleotides in length.

9. The particle of claim 1, wherein the genetic element binds the ORF1 molecule.

10. The particle of claim 1, wherein the particle comprises a plurality of genetic elements, e.g., at least 2, 3, 4, 5, 10, 20, 30, 40, 50, or 60 genetic elements.

11. The particle of claim 10, wherein the genetic elements of the plurality each comprise the same sequence.

12. The particle of claim 1, wherein the genetic element comprises an mRNA cap and/or a poly-A tail.

13. The particle of claim 12, wherein the poly-A tail is at least about 5, 10, 15, 20, 25, 30, 40, 50, 60, 70, 80, 90, or 100 adenosines in length.

14. The particle of claim 1, wherein the ORF1 molecule comprises an amino acid sequence of any one of SEQ ID NOs: 21, 58, 891, 1005, or 1012, or an amino acid sequence having at least 30%, 40%, 50%, 60%, 70%, 75%, 80%, 85%, 90%, 95%, 96%, 97%, 98%, or 99% sequence identity thereto.

15. The particle of claim 1, wherein the genetic element:
(i) lacks a sequence encoding an Anellovirus ORF1 protein;
(ii) lacks a sequence encoding an Anellovirus ORF2 protein; and/or
(iii) lacks a sequence encoding an Anellovirus ORF3 protein.

16. The particle of claim 1, wherein the genetic element comprises a 5′ UTR.

17. A composition comprising a plurality of the particles of claim 1.

18. A pharmaceutical composition comprising the composition of claim 17, and a pharmaceutically acceptable carrier or excipient.

19. A method of making a particle according to claim 1, the method comprising:
(a) providing a mixture comprising:
(i) a genetic element comprising mRNA, and
(ii) an ORF1 molecule; and
(b) incubating the mixture under conditions suitable for enclosing the genetic element within a proteinaceous exterior comprising the ORF1 molecule, thereby making the particle.

20. The method of claim 19, wherein the mixture is not comprised in a cell.

21. A method of purifying a particle, the method comprising:
(a) providing a particle of claim 1; and
(b) purifying the particle.

22. A method of manufacturing a pharmaceutical composition, the method comprising:
providing a plurality of particles according to claim 1; and
formulating the plurality of particles as a pharmaceutical composition suitable for administration to a subject.

23. The method of claim 22, further comprising, between the providing step and the formulating step, evaluating the plurality of particles for one or more of:
(i) a contaminant,
(ii) an optical density measurement,
(iii) particle number, and/or
(iv) infectivity.

24. The method of claim 23, wherein the pharmaceutical composition is suitable for administration to a subject if one or more of (i)-(iv) meet a specified threshold.

25. A method of treating a disease or disorder in a subject in need thereof, the method comprising administering to the subject the particle of claim 1, thereby treating a disease or disorder (e.g., as described herein) in the subject.

26. A method of modulating, e.g., enhancing or inhibiting, a biological function (e.g., as described herein) in a subject, the method comprising administering the particle of claim 1 to the subject.

27. A method of delivering a genetic element to a cell, the method comprising contacting the particle of claim 1 with a cell.

28. The method of claim 27, wherein the cell is a human cell.

29. The method of claim 27, wherein the cell is a bone marrow cell, blood cell, heart cell, GI cell, skin cell, retinal cell, liver cell, cell of the spinal cord, motor neuron, myocyte, cell of an epithelial lining, or pancreatic cell.


SEQUENCE LISTING (968 sequences)

SEQ ID NO: 1 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 2 - protein, 688 aa, Artificial Sequence
  Description of Artificial Sequence: Synthetic polypeptide
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 3 - protein, 714 aa, Artificial Sequence
  Description of Artificial Sequence: Synthetic polypeptide
MGSSHHHHHHGSDYKDDDDKSGSLEVLFQGPSGMPPYWRQKYYRRRYRPFSWRTRRIIQRRKRWRYRKPRKTYWRRKLRVRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 4 - protein, 714 aa, Artificial Sequence
  Description of Artificial Sequence: Synthetic polypeptide
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 5 - protein, 688 aa, Artificial Sequence
  Description of Artificial Sequence: Synthetic polypeptide
MGSSHHHHHHGSDYKDDDDKSGSLEVLFQGPSGMPPYWRQKYYRRRYRPFSWRTRRIIQRRHRWRYRKPRHTYWRRHLRVRHRFYHRHLHHIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 6 - protein, 687 aa, Artificial Sequence
  Description of Artificial Sequence: Synthetic polypeptide
MGSSHHHHHHGSDYKDDDDKSGSLEVLFQGPSGMWGTSNCACAKFQIRRRYARPYRRRHIRRYRRRRRHFRRRRFTTNRRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 7 - protein, 688 aa, Artificial Sequence
  Description of Artificial Sequence: Synthetic polypeptide
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHAKKWFSRETRKKRKPGFKRLLGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 8 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRRCTIFGTICLFQGSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 9 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSPERANNNYIQTIYSYVPDKEPGGGGWTLITESLSSLWEDWEHLKNVWTQSNAGLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 10 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYGGVTLYFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 11 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHNCYPMTDTKYTHADSAPNRMLLKKHKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 12 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYKRVRVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 13 - protein, 699 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKHVIRVPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 14 - protein, 673 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 15 - protein, 673 aa, Artificial Sequence
  Description of Artificial Sequence: Synthetic polypeptide
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 16 - DNA, 3753 bp, Alphatorquevirus sp.
tgctacgtcactaacccacgtgtcctctacaggccaatcgcagtctatgtcgtgcacttcctgggcatggtctacataattatataaatgcttgcacttccgaatggctgagtttttgctgcccgtccgcggagaggagccacggcaggggatccgaacgtcctgagggcgggtgccggaggtgagtttacacaccgaagtcaaggggcaattcgggctcaggactggccgggctttgggcaaggctcttaaaaatgcacttttctcgaataagcagaaagaaaaggaaagtgctactgctttgcgtgccagcagctaagaaaaaaccaactgctatgagcttctggaaacctccggtacacaatgtcacggggatccaacgcatgtggtatgagtcctttcaccgtggccacgcttctttttgtggttgtgggaatcctatacttcacattactgcacttgctgaaacatatggccatccaacaggcccgagaccttctgggccaccgggagtagaccccaacccccacatccgtagagccaggcctgccccggccgctccggagccctcacaggttgattcgagaccagccctgacatggcatggggatggtggaagcgacggaggcgctggtggttccggaagcggtggacccgtggcagacttcgcagacgatggcctcgatcagctcgtcgccgccctagacgacgaagagtaaggaggcgcagacggtggaggagggggagacgaaaaacaaggacttacagacgcaggagacgctttagacgcaggggacgaaaagcaaaacttataataaaactgtggcaacctgcagtaattaaaagatgcagaataaagggatacataccactgattataagtgggaacggtacctttgccacaaactttaccagtcacataaatgacagaataatgaaaggccccttcgggggaggacacagcactatgaggttcagcctctacattttgtttgaggagcacctcagacacatgaacttctggaccagaagcaacgataacctagagctaaccagatacttgggggcttcagtaaaaatatacaggcacccagaccaagactttatagtaatatacaacagaagaacccctctaggaggcaacatctacacagcaccctctctacacccaggcaatgccattttagcaaaacacaaaatattagtaccaagtttacagacaagaccaaagggtagaaaagcaattagactaagaatagcaccccccacactctttacagacaagtggtactttcaaaaggacatagccgacctcacccttttcaacatcatggcagttgaggctgacttgcggtttccgttctgctcaccacaaactgacaacacttgcatcagcttccaggtccttagttccgtttacaacaactacctcagtattaatacctttaataatgacaactcagactcaaagttaaaagaatttttaaataaagcatttccaacaacaggcacaaaaggaacaagtttaaatgcactaaatacatttagaacagaaggatgcataagtcacccacaactaaaaaaaccaaacccacaaataaacaaaccattagagtcacaatactttgcacctttagatgccctctggggagaccccatatactataatgatctaaatgaaaacaaaagtttgaacgatatcattgagaaaatactaataaaaaacatgattacataccatgcaaaactaagagaatttccaaattcataccaaggaaacaaggccttttgccacctaacaggcatatacagcccaccatacctaaaccaaggcagaatatctccagaaatatttggactgtacacagaaataatttacaacccttacacagacaaaggaactggaaacaaagtatggatggacccactaactaaagagaacaacatatataaagaaggacagagcaaatgcctactgactgacatgcccctatggactttactttttggatatacagactggtgtaaaaaggacactaataactgggacttaccactaaactacagactagtactaatatgcccttatacctttccaaaattgtacaatgaaaaagtaaaagactatgggtacatcccgtactcctacaaattcggagcgggtcagatgccagacggcagcaactacataccctttcagtttagagcaaagtggtaccccacagtactacaccagcaacaggtaatggaggacataagcaggagcgggccctttgcacctaaggtagaaaaaccaagcactcagctggtaatgaagtactgttttaactttaactggggcggtaaccctatcattgaacagattgttaaagaccccagcttccagcccacctatgaaatacccggtaccggtaacatccctagaagaatacaagtcatcgacccgcgggtcctgggaccgcactactcgttccggtcatgggacatgcgcagacacacatttagcagagcaagtattaagagagtgtcagaacaacaagaaacttctgaccttgtattctcaggcccaaaaaagcctcgggtcgacatcccaaaacaagaaacccaagaagaaagctcacattcactccaaagagaatcgagaccgtgggagaccgaggaagaaagcgagacagaagccctctcgcaagagagccaagaggtccccttccaacagcagttgcagcagcagtaccaagagcagctcaagctcagacagggaatcaaagtcctcttcgagcagctcataaggacccaacaaggggtccatgtaaacccatgcctacggtaggtcccaggcagtggctgtttccagagagaaagccagccccagctcctagcagtggagactgggccatggagtttctcgcagcaaaaatatttgataggccagttagaagcaaccttaaagataccccttactacccatatgttaaaaaccaatacaatgtctactttgaccttaaatttgaataaacagcagcttcaaacttgcaaggccgtgggagtttcactggtcggtgtctacctctaaaggtcactaagcactccgagcgtaagcgaggagtgcgaccctcccccctggaacaacttcttcggagtccggcgctacgccttcggctgcgccggacacctcagaccccccctccacccgaaacgcttgcgcgtttcggaccttcggcgtcgggggggtcgggagctttattaaacggactccgaagtgctcttggacactgagggggtgaacagcaacgaaagtgagtggggccagacttcgccataaggcctttatcttcttgccatttgtcagtgtccggggtcgccataggcttcgggctcgtttttaggccttccggactacaaaaatcgccattttggtgacgtcacggccgccatcttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcctctttttcaaaaaaaagcggaagtgccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 17 - protein, 127 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEE

SEQ ID NO: 18 - protein, 268 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEELLKTPASSPPMKYPVPVTSLEEYKSSTRGSWDRTTRSGHGTCADTHLAEQVLRECQNNKKLLTLYSQAQKSLGSTSQNKKPKKKAHIHSKENRDRGRPRKKARQKPSRKRAKRSPSNSSCSSSTKSSSSSDRESKSSSSSS

SEQ ID NO: 19 - protein, 276 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEEPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 20 - protein, 167 aa, Alphatorquevirus sp.
MSFWKPPVHNVTGIQRMWPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 21 - protein, 743 aa, Alphatorquevirus sp.
MAWGWWKRRRRWWFRKRWTRGRLRRRWPRSARRRPRRRRVRRRRRWRRGRRKTRTYRRRRRFRRRGRKAKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSINTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGRISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFNWGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 22 - protein, 194 aa, Alphatorquevirus sp.
MAWGWWKRRRRWWFRKRWTRGRLRRRWPRSARRRPRRRRIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 23 - protein, 113 aa, Alphatorquevirus sp.
MAWGWWKRRRRWWFRKRWTRGRLRRRWPRSARRRPRRRRAQKSLGSTSQNKKPKKKAHIHSKENRDRGRPRKKARQKPSRKRAKRSPSNSSCSSSTKSSSSSDRESKSSSSSS

SEQ ID NO: 24-53 - skipped (30 entries)

SEQ ID NO: 54 - DNA, 2979 bp, Betatorquevirus sp.
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61-104 - skipped (44 entries)

SEQ ID NO: 105 - DNA, 71 bp, Artificial Sequence
  Description of Artificial Sequence: Synthetic oligonucleotide
cgggtgccgkaggtgagtttacacaccgmagtcaaggggcaattcgggctcrggactggccgggcyhtggg

SEQ ID NO: 106 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctwtggg

SEQ ID NO: 107 - DNA, 71 bp, Artificial Sequence
  Description of Artificial Sequence: Synthetic oligonucleotide
cgggtgccgtaggtgagtttacacaccgcagtcaaggggcaattcgggctcgggactggccgggctatggg

SEQ ID NO: 108 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccctggg

SEQ ID NO: 109 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctttggg

SEQ ID NO: 110 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctatggg

SEQ ID NO: 111 - DNA, 71 bp, Artificial Sequence
  Description of Artificial Sequence: Synthetic oligonucleotide
cgggtgccggaggtgagtttacacaccgaagtcaaggggcaattcgggctcaggactggccgggctttggg

SEQ ID NO: 112 - DNA, 71 bp, Artificial Sequence
  Description of Artificial Sequence: Synthetic oligonucleotide
cgggtgccggaggtgagtttacacaccgcagtcaaggggcaattcgggctcgggactggccgggcyhtggg

SEQ ID NO: 113 - DNA, 71 bp, Artificial Sequence; identical to SEQ ID NO: 107
  Description of Artificial Sequence: Synthetic oligonucleotide

SEQ ID NO: 114 - DNA, 70 bp, Artificial Sequence
  Description of Artificial Sequence: Synthetic oligonucleotide
cgggtgccggaggtgagtttacacaccgcagtcaaggggcaattcgggctcgggactggccgggcccggg

SEQ ID NO: 115 - DNA, 71 bp, Artificial Sequence; identical to SEQ ID NO: 111
  Description of Artificial Sequence: Synthetic oligonucleotide

SEQ ID NO: 116 - DNA, 69 bp, Artificial Sequence
  Description of Artificial Sequence: Synthetic oligonucleotide
cgggtgccggaggtgagtttacacaccgcagtcaaggggcaattcgggctcgggaggccgggccatggg

SEQ ID NO: 117 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccccggg

SEQ ID NO: 118 - DNA, 71 bp, Artificial Sequence; identical to SEQ ID NO: 110
  Description of Artificial Sequence: Synthetic oligonucleotide

SEQ ID NO: 119 - DNA, 71 bp, Artificial Sequence
  Description of Artificial Sequence: Synthetic oligonucleotide
cgggtgccgaaggtgagtttacacaccgcagtcaaggggcaattcgggctcgggactggccgggctatggg

SEQ ID NO: 120 - DNA, 117 bp, Artificial Sequence
  Description of Artificial Sequence: Synthetic polynucleotide
  misc_feature (10)..(10): May or may not be present
  misc_feature (12)..(12): May or may not be present
  misc_feature (30)..(32): May or may not be present
  misc_feature (34)..(34): May or may not be present
  misc_feature (43)..(46): May or may not be present
  misc_feature (52)..(54): May or may not be present
  misc_feature (70)..(71): May or may not be present
  misc_feature (89)..(90): May or may not be present
  misc_feature (103)..(103): May or may not be present
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121 - DNA, 169 bp, Artificial Sequence
  Description of Artificial Sequence: Synthetic polynucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
  modified_base (97)..(98): a, c, t, g, unknown or other
gccgccgcggcggcggsggngnsgcgcgctdcgcgcgcsnnncrccrgggggnnnncwgcsncncccccccccgcgcatgcgcgggkcccccccccnncggggggctccgccccccggcccccccccgtgctaaacccaccgcgcatgcgcgaccacgcccccgccgcc

SEQ ID NO: 122 - DNA, 79 bp, Artificial Sequence
  Description of Artificial Sequence: Synthetic oligonucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
gccgccgcggcggcggsggngnsgcgcgctdcgcgcgcsnnncrccrgggggnnnncwgcsncncccccccccgcgcat

SEQ ID NO: 123 - DNA, 31 bp, Artificial Sequence
  Description of Artificial Sequence: Synthetic oligonucleotide
  modified_base (18)..(19): a, c, t, g, unknown or other
gcgcgggkcccccccccnncggggggctccg

SEQ ID NO: 124 - DNA, 59 bp, Artificial Sequence
  Description of Artificial Sequence: Synthetic oligonucleotide
ccccccggcccccccccgtgctaaacccaccgcgcatgcgcgaccacgcccccgccgcc

SEQ ID NO: 125 - DNA, 156 bp, Artificial Sequence
  Description of Artificial Sequence: Synthetic polynucleotide
gcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 126 - DNA, 7 bp, Artificial Sequence
  Description of Artificial Sequence: Synthetic oligonucleotide
gcggcgg

SEQ ID NO: 127 - DNA, 7 bp, Artificial Sequence
  Description of Artificial Sequence: Synthetic oligonucleotide
gggggcg

SEQ ID NO: 128 - DNA, 6 bp, Artificial Sequence
  Description of Artificial Sequence: Synthetic oligonucleotide
gccgcg

SEQ ID NO: 129 - DNA, 25 bp, Artificial Sequence
  Description of Artificial Sequence: Synthetic oligonucleotide
ttcgcgcgccgcccaccagggggtg

SEQ ID NO: 130 - DNA, 5 bp, Artificial Sequence
  Description of Artificial Sequence: Synthetic oligonucleotide
ctgcg

SEQ ID NO: 131 - DNA, 17 bp, Artificial Sequence
  Description of Artificial Sequence: Synthetic oligonucleotide
cgcccccccccgcgcat

SEQ ID NO: 132 - DNA, 17 bp, Artificial Sequence
  Description of Artificial Sequence: Synthetic oligonucleotide
gcgcggggccccccccc

SEQ ID NO: 133 - DNA, 72 bp, Artificial Sequence
  Description of Artificial Sequence: Synthetic oligonucleotide
gggggggctccgcccccccggcccccccccgtgctaaacccaccgcgcatgcgcgaccacgcccccgccgcc

SEQ ID NO: 134 - DNA, 115 bp, Artificial Sequence
  Description of Artificial Sequence: Synthetic polynucleotide
cggcggcggcggcgcgcgcgctgcgcgcgcgcgccgggggggcgccagcgcccccccccccgcgcatgcacgggtccccccccccacggggggctccgccccccggccccccccc

SEQ ID NO: 135 - DNA, 14 bp, Artificial Sequence
  Description of Artificial Sequence: Synthetic oligonucleotide
cggcggcggcggcg

SEQ ID NO: 136 - DNA, 17 bp, Artificial Sequence
  Description of Artificial Sequence: Synthetic oligonucleotide
cgcgcgctgcgcgcgcg

SEQ ID NO: 137 - DNA, 19 bp, Artificial Sequence
  Description of Artificial Sequence: Synthetic oligonucleotide
cgccgggggggcgccagcg

SEQ ID NO: 138 - DNA, 17 bp, Artificial Sequence
  Description of Artificial Sequence: Synthetic oligonucleotide
cccccccccccgcgcat

SEQ ID NO: 139 - DNA, 31 bp, Artificial Sequence
  Description of Artificial Sequence: Synthetic oligonucleotide
gcacgggtccccccccccacggggggctccg

SEQ ID NO: 140 - DNA, 17 bp, Artificial Sequence
  Description of Artificial Sequence: Synthetic oligonucleotide
ccccccggccccccccc

SEQ ID NO: 141 - DNA, 121 bp, Artificial Sequence
  Description of Artificial Sequence: Synthetic polynucleotide
ccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 142 - DNA, 37 bp, Artificial Sequence
  Description of Artificial Sequence: Synthetic oligonucleotide
ccgtcggcgggggggccgcgcgctgcgcgcgcggccc

SEQ ID NO: 143 - DNA, 84 bp, Artificial Sequence
  Description of Artificial Sequence: Synthetic oligonucleotide
ccgggggaggcacagcctcccccccccgcgcgcatgcgcgcgggtccccccccctccggggggctccgccccccggcccccccc

SEQ ID NO: 144 - DNA, 104 bp, Artificial Sequence
  Description of Artificial Sequence: Synthetic polynucleotide
cggcggcggcgcgcgcgctacgcgcgcgcgccggggggctgccgccccccccccgcgcatgcgcggggcccccccccgcggggggctccgccccccggcccccc

SEQ ID NO: 145 - DNA, 11 bp, Artificial Sequence
  Description of Artificial Sequence: Synthetic oligonucleotide
cggcggcggcg

SEQ ID NO: 146 - DNA, 17 bp, Artificial Sequence
  Description of Artificial Sequence: Synthetic oligonucleotide
cgcgcgctacgcgcgcg

SEQ ID NO: 147 - DNA, 10 bp, Artificial Sequence
  Description of Artificial Sequence: Synthetic oligonucleotide
cgccgggggg

SEQ ID NO: 148 - DNA, 7 bp, Artificial Sequence
  Description of Artificial Sequence: Synthetic oligonucleotide
ctgccgc

SEQ ID NO: 149 - DNA, 15 bp, Artificial Sequence
  Description of Artificial Sequence: Synthetic oligonucleotide
cccccccccgcgcat

SEQ ID NO: 150 - DNA, 17 bp, Artificial Sequence
  Description of Artificial Sequence: Synthetic oligonucleotide
gcgcggggccccccccc

SEQ ID NO: 151 - DNA, 13 bp, Artificial Sequence
  Description of Artificial Sequence: Synthetic oligonucleotide
gcggggggctccg

SEQ ID NO: 152 - DNA, 14 bp, Artificial Sequence
  Description of Artificial Sequence: Synthetic oligonucleotide
ccccccggcccccc

SEQ ID NO: 153 - DNA, 122 bp, Artificial Sequence
  Description of Artificial Sequence: Synthetic polynucleotide
gccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 154 - DNA, 19 bp, Artificial Sequence
  Description of Artificial Sequence: Synthetic oligonucleotide
gccgccgcggcggcggggg

SEQ ID NO: 155 - DNA, 41 bp, Artificial Sequence
  Description of Artificial Sequence: Synthetic oligonucleotide
gcggcgcgctgcgcgcgccgcccagtagggggagccatgcg

SEQ ID NO: 156 - DNA, 15 bp, Artificial Sequence
  Description of Artificial Sequence: Synthetic oligonucleotide
cccccccccgcgcat

SEQ ID NO: 157 - DNA, 17 bp, Artificial Sequence
  Description of Artificial Sequence: Synthetic oligonucleotide
gcgcggggccccccccc

SEQ ID NO: 158 - DNA, 13 bp, Artificial Sequence
  Description of Artificial Sequence: Synthetic oligonucleotide
gcggggggctccg

SEQ ID NO: 159 - DNA, 17 bp, Artificial Sequence
  Description of Artificial Sequence: Synthetic oligonucleotide
ccccccggccccccccg

SEQ ID NO: 160 - DNA, 36 bp, Artificial Sequence
  Description of Artificial Sequence: Synthetic oligonucleotide
cgcgctgcgcgcgccgcccagtagggggagccatgc

SEQ ID NO: 161 - DNA, 78 bp, Artificial Sequence
  Description of Artificial Sequence: Synthetic oligonucleotide
ccgccatcttaagtagttgaggcggacggtggcgtgagttcaaaggtcaccatcagccacacctactcaaaatggtgg

SEQ ID NO: 162 - DNA, 172 bp, Artificial Sequence
  Description of Artificial Sequence: Synthetic polynucleotide
cttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcc

SEQ ID NO: 163 - DNA, 36 bp, Artificial Sequence; identical to SEQ ID NO: 160
  Description of Artificial Sequence: Synthetic oligonucleotide

SEQ ID NO: 164 - DNA, 36 bp, Artificial Sequence
  Description of Artificial Sequence: Synthetic oligonucleotide
gcgctdcgcgcgcgcgccggggggctgcgccccccc

SEQ ID NO: 165 - DNA, 36 bp, Artificial Sequence
  Description of Artificial Sequence: Synthetic oligonucleotide
gcgcttcgcgcgccgcccactagggggcgttgcgcg

SEQ ID NO: 166 - DNA, 36 bp, Artificial Sequence
  Description of Artificial Sequence: Synthetic oligonucleotide
gcgctgcgcgcgccgcccagtagggggcgcaatgcg

SEQ ID NO: 167 - DNA, 36 bp, Artificial Sequence
  Description of Artificial Sequence: Synthetic oligonucleotide
gcgctgcgcgcgcggcccccgggggaggcattgcct

SEQ ID NO: 168 - DNA, 36 bp, Artificial Sequence
  Description of Artificial Sequence: Synthetic oligonucleotide
gcgctgcgcgcgcgcgccgggggggcgccagcgccc

SEQ ID NO: 169 - DNA, 36 bp, Artificial Sequence
  Description of Artificial Sequence: Synthetic oligonucleotide
gcgcttcgcgcgcgcgccggggggctccgccccccc

SEQ ID NO: 170 - DNA, 36 bp, Artificial Sequence
  Description of Artificial Sequence: Synthetic oligonucleotide
gcgcttcgcgcgcgcgccggggggctgcgccccccc

SEQ ID NO: 171 - DNA, 36 bp, Artificial Sequence
  Description of Artificial Sequence: Synthetic oligonucleotide
gcgctacgcgcgcgcgccggggggctgcgccccccc

SEQ ID NO: 172 - DNA, 36 bp, Artificial Sequence
  Description of Artificial Sequence: Synthetic oligonucleotide
gcgctacgcgcgcgcgccggggggctctgccccccc

SEQ ID NO: 173-184 - skipped (12 entries)

SEQ ID NO: 185 - protein, 743 aa, Alphatorquevirus sp.; identical to SEQ ID NO: 21

SEQ ID NO: 186 - protein, 68 aa, Alphatorquevirus sp.
MAWGWWKRRRRWWFRKRWTRGRLRRRWPRSARRRPRRRRVRRRRRWRRGRRKTRTYRRRRRFRRRGRK

SEQ ID NO: 187 - protein, 212 aa, Alphatorquevirus sp.
AKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSI

SEQ ID NO: 188 - protein, 133 aa, Alphatorquevirus sp.
NTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGR

SEQ ID NO: 189 - protein, 166 aa, Alphatorquevirus sp.
ISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFN

SEQ ID NO: 190 - protein, 164 aa, Alphatorquevirus sp.
WGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 191-214 - skipped (24 entries)

SEQ ID NO: 215 - protein, 666 aa, Betatorquevirus sp.; identical to SEQ ID NO: 58

SEQ ID NO: 216 - protein, 38 aa, Betatorquevirus sp.
MPYYYRRRRYNYRRPRWYGRGWIRRPFRRRFRRKRRVR

SEQ ID NO: 217 - protein, 208 aa, Betatorquevirus sp.
PTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 218 - protein, 128 aa, Betatorquevirus sp.
ISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAG

SEQ ID NO: 219 - protein, 163 aa, Betatorquevirus sp.
TMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFK

SEQ ID NO: 220 - protein, 129 aa, Betatorquevirus sp.
WGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 221-226 - skipped (6 entries)

SEQ ID NO: 227 - protein, 220 aa, Artificial Sequence
  Description of Artificial Sequence: Synthetic polypeptide
  MOD_RES (29)..(31): Any amino acid
  SITE (29)..(31): This region may encompass 0-3 residues
  MOD_RES (100)..(100): Any amino acid
  MOD_RES (125)..(129): Any amino acid
  SITE (125)..(129): This region may encompass 1-5 residues
  MOD_RES (181)..(181): Any amino acid
  MOD_RES (211)..(211): Any amino acid
LVLTQWQPNTVRRCYIRGYLPLIICGENXXXTTSRNYATHSDDTIQKGPFGGGMSTTTFSLRVLYDEYQRFMNRWTYSNEDLDLARYLGCKFTFYRHPDXDFIVQYNTNPPFKDTKLTAPSIHPXXXXXGMLMLSKRKILIPSLKTRPKGKHYVKVRIGPPKLFEDKWYTQSDLCDVPLVXLYATAADLQHPFGSPQTDNPCVTFQVLGSXYNKHLSISP

SEQ ID NO: 228 - protein, 172 aa, Artificial Sequence
  Description of Artificial Sequence: Synthetic polypeptide
  MOD_RES (38)..(38): Any amino acid
  MOD_RES (44)..(46): Any amino acid
  SITE (44)..(46): This region may encompass 0-3 residues
  MOD_RES (77)..(77): Any amino acid
  MOD_RES (79)..(79): Any amino acid
  MOD_RES (98)..(101): Any amino acid
  SITE (98)..(101): This region may encompass 0-4 residues
SNFEFPGAYTDITYNPLTDKGVGNMVWIQYLTKPDTIXDKTQSXXXKCLIEDLPLWAALYGYVDFCEKETGDSAIIXNXGRVLIRCPYTKPPLYDKTXXXXNKGFVPYSTNFGNGKMPGGSGYVPIYWRARWYPTLFHQKEVLEDIVQSGPFAYKDEKPSTQLVMKYCFNFN

SEQ ID NO: 229 - protein, 258 aa, Artificial Sequence
  Description of Artificial Sequence: Synthetic polypeptide
  MOD_RES (20)..(22): Any amino acid
  SITE (20)..(22): This region may encompass 0-3 residues
  MOD_RES (25)..(25): Any amino acid
  MOD_RES (78)..(78): Any amino acid
  MOD_RES (89)..(89): Any amino acid
  MOD_RES (91)..(91): Any amino acid
  MOD_RES (95)..(98): Any amino acid
  SITE (95)..(98): This region may encompass 1-4 residues
  MOD_RES (107)..(120): Any amino acid
  SITE (107)..(120): This region may encompass 2-14 residues
  MOD_RES (129)..(129): Any amino acid
  MOD_RES (139)..(168): Any amino acid
  SITE (139)..(168): This region may encompass 0-30 residues
  MOD_RES (201)..(204): Any amino acid
  SITE (201)..(204): This region may encompass 0-4 residues
  MOD_RES (219)..(258): Any amino acid
  SITE (219)..(258): This region may encompass 0-40 residues
WGGNPISQQVVRNPCKDSGXXXSGXGRQPRSVQVVDPKYMGPEYTFHSWDWRRGLFGEKAIKRMSEQPTDDEIFTGGXPKRPRRDPPTXQXPEEXXXXQKESSSFRXXXXXXXXXXXXXXPWESSSQEXESESQEEEEXXXXXXXXXXXXXXXXXXXXXXXXXXXXXXEQTVQQQLRQQLREQRRLRVQLQLLFQQLLKTXXXXQAGLHINPLLLSQAXXXXXXXXXXXXXXXXXXXXXXXXXXXXXXXXXXXXXXXX

SEQ ID NO: 230 - protein, 214 aa, Artificial Sequence
  Description of Artificial Sequence: Synthetic polypeptide
  MOD_RES (136)..(136): Any amino acid
  MOD_RES (138)..(141): Any amino acid
  SITE (138)..(141): This region may encompass 1-4 residues
  MOD_RES (179)..(179): Any amino acid
LKQWQPSTIRKCKIKGYLPLFQCGKGRISNNYTQYKESIVPHHEPGGGGWSIQQFTLGALYEEHLKLRNWWTKSNDGLPLVRYLGCTIKLYRSEDTDYIVTYQRCYPMTATKLTYLSTQPSRMLMNKHKIIVPSKXTXXXXNKKKKPYKKIFIKPPSQMQNKWYFQQDIANTPLLQLTXTACSLDRMYLSSDSISNNITFTSLNTNFFQNPNFQ

SEQ ID NO: 231 - protein, 187 aa, Artificial Sequence
  Description of Artificial Sequence: Synthetic polypeptide
  MOD_RES (1)..(10): Any amino acid
  SITE (1)..(10): This region may encompass 4-10 residues
  MOD_RES (38)..(45): Any amino acid
  SITE (38)..(45): This region may encompass 1-8 residues
  MOD_RES (94)..(94): Any amino acid
  MOD_RES (100)..(102): Any amino acid
  SITE (100)..(102): This region may encompass 1-3 residues
  MOD_RES (112)..(112): Any amino acid
  MOD_RES (114)..(115): Any amino acid
  SITE (114)..(115): This region may encompass 0-2 residues
  MOD_RES (124)..(139): Any amino acid
  SITE (124)..(139): This region may encompass 3-16 residues
  MOD_RES (154)..(154): Any amino acid
XXXXXXXXXXTPLYFECRYNPFKDKGTGNKVYLVSNNXXXXXXXXTGWDPPTDPDLIIEGFPLWLLLWGWLDWQKKLGKIQNIDTDYILVIQSXYYIPPXXXKLPYYVPLDXDXXFLHGRSPYXXXXXXXXXXXXXXXXPSDKQHWHPKVRFQXETINNIALTGPGTPKLPNQKSIQAHMKYKFYFK

SEQ ID NO: 232 - protein, 163 aa, Artificial Sequence
  Description of Artificial Sequence: Synthetic polypeptide
  MOD_RES (34)..(34): Any amino acid
  MOD_RES (65)..(65): Any amino acid
  MOD_RES (77)..(78): Any amino acid
  MOD_RES (86)..(87): Any amino acid
  MOD_RES (96)..(96): Any amino acid
  MOD_RES (102)..(106): Any amino acid
  SITE (102)..(106): This region may encompass 0-5 residues
  MOD_RES (125)..(125): Any amino acid
  MOD_RES (135)..(135): Any amino acid
  MOD_RES (138)..(163): Any amino acid
  SITE (138)..(163): This region may encompass 0-26 residues
WGGCPAPMETITDPCKQPKYPIPNNLLQTTSLQXPTTPIETYLYKFDERRGLLTKKAAKRIKKDXTTETTLFTDTGXXTSTTLPTXXQTETTQEEXTSEEEXXXXXETLLQQLQQLRRKQKQLRXRILQLLQLLXLLXXXXXXXXXXXXXXXXXXXXXXXXXX

SEQ ID NO: 233 - protein, 203 aa, Artificial Sequence
  Description of Artificial Sequence: Synthetic polypeptide
  MOD_RES (79)..(79): Any amino acid
  MOD_RES (104)..(104): Any amino acid
  MOD_RES (116)..(116): Any amino acid
  MOD_RES (120)..(121): Any amino acid
  MOD_RES (125)..(125): Any amino acid
  MOD_RES (170)..(170): Any amino acid
TIPLKQWQPESIRKCKIKGYGTLVLGAEGRQFYCYTNEKDEYTPPKAPGGGGFGVELFSLEYLYEQWKARNNIWTKSNXYKDLCRYTGCKITFYRHPTTDFIVXYSRQPPFEIDKXTYMXXHPQXLLLRKHKKIILSKATNPKGKLKKKIKIKPPKQMLNKWFFQKQFAXYGLVQLQAAACBLRYPRLGCCNENRLITLYYLN

SEQ ID NO: 234 - protein, 162 aa, Artificial Sequence
  Description of Artificial Sequence: Synthetic polypeptide
  MOD_RES (12)..(12): Any amino acid
  MOD_RES (20)..(20): Any amino acid
  MOD_RES (23)..(23): Any amino acid
  MOD_RES (30)..(30): Any amino acid
  MOD_RES (58)..(58): I or L
  MOD_RES (84)..(84): Any amino acid
  MOD_RES (90)..(90): Any amino acid
  MOD_RES (95)..(95): Any amino acid
  MOD_RES (105)..(105): Any amino acid
  MOD_RES (111)..(111): I or L
  MOD_RES (113)..(113): Any amino acid
  MOD_RES (154)..(154): Any amino acid
  MOD_RES (156)..(156): Any amino acid
LPIVVARYNPAXDTGKGNKXWLXSTLNGSXWAPPTTDKDLIIEGLPLWLALYGYWSYXKKVKKDKGILQSHMFVVKSPAIQPLXTATTQXTFYPXIDNSFIQGKXPYDEPXTXNQKKLWYPTLEHQQETINAIVESGPYVPKLDNQKNSTWELXYXYTFYFK

SEQ ID NO: 235 - protein, 177 aa, Artificial Sequence
  Description of Artificial Sequence: Synthetic polypeptide
  MOD_RES (16)..(16): Any amino acid
  MOD_RES (26)..(26): Any amino acid
  MOD_RES (33)..(33): Any amino acid
  MOD_RES (73)..(73): Any amino acid
  MOD_RES (81)..(82): Any amino acid
  SITE (81)..(82): This region may encompass 0-2 residues
  MOD_RES (90)..(90): Any amino acid
  MOD_RES (94)..(94): Any amino acid
  MOD_RES (119)..(124): Any amino acid
  SITE (119)..(124): This region may encompass 1-6 residues
  MOD_RES (168)..(177): Any amino acid
  SITE (168)..(177): This region may encompass 1-10 residues
WGGPQIPDQPVEDPKXQGTYPVPDTXQQTIQIXNPLKQKPETMFHDWDYRRGIITSTALKRMQENLETDSSFXSDSEETPXXKKKKRLTXELPXPQEETEEIQSCLLSLCEESTCQEEXXXXXXENLQQLIHQQQQQQQQLKHNILKLLSDLKZKQRLLQLQTGILEXXXXXXXXXX

SEQ ID NO: 236-800 - skipped (565 entries)

SEQ ID NO: 801 - DNA, 156 bp, Artificial Sequence; identical to SEQ ID NO: 125
  Description of Artificial Sequence: Synthetic polynucleotide

SEQ ID NO: 802 - DNA, 150 bp, Artificial Sequence
  Description of Artificial Sequence: Synthetic polynucleotide
ccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 803 - DNA, 122 bp, Artificial Sequence; identical to SEQ ID NO: 153
  Description of Artificial Sequence: Synthetic polynucleotide

SEQ ID NO: 804 - DNA, 111 bp, Artificial Sequence
  Description of Artificial Sequence: Synthetic polynucleotide
cggcccagcggcggcgcgcgcgcttcgcgcgcgcgccggggggctccgcccccccccgcgcatgcgcggggcccccccccgcggggggctccgccccccggtccccccccg

SEQ ID NO: 805 - DNA, 115 bp, Artificial Sequence
  Description of Artificial Sequence: Synthetic polynucleotide
cggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 806 - DNA, 104 bp, Artificial Sequence; identical to SEQ ID NO: 144
  Description of Artificial Sequence: Synthetic polynucleotide

SEQ ID NO: 807 - DNA, 108 bp, Artificial Sequence
  Description of Artificial Sequence: Synthetic polynucleotide
ggcggcggcgcgcgcgctacgcgcgcgcgccggggagctctgcccccccccgcgcatgcgcgcgggtcccccccccgcggggggctccgccccccggtcccccccccg

SEQ ID NO: 808-828 - skipped (21 entries)

SEQ ID NO: 829 - protein, 11 aa, Artificial Sequence
  Description of Artificial Sequence: Synthetic peptide
  MOD_RES (4)..(5): Any amino acid
  MOD_RES (7)..(7): Any amino acid
  MOD_RES (9)..(10): Any amino acid
YNPXXDXGXXN

SEQ ID NO: 830-885 - skipped (56 entries)

SEQ ID NO: 886 - DNA, 3176 bp, Gammatorquevirus sp.
taaaatggcgggagccaatcattttatactttcactttccaattaaaaatggccacgtcacaaacaaggggtggagccatttaaactatataactaagtggggtggcgaatggctgagtttaccccgctagacggtgcagggaccggatcgagcgcagcgaggaggtccccggctgcccatgggcgggagccgaggtgagtgaaaccaccgaggtctaggggcaattcgggctagggcagtctagcggaacgggcaagaaacttaaaacaatatttgttttacagatggttagtatatcctcaagtgatttttttaagaaaacgaaatttaatgaggagacgcagaaccaagtatggatgtctcaaattgctgactctcatgataatatctgcagttgctggcatccatttgctcaccttcttgcttccatatttcctcctggccacaaagatcgtgatcttactattaaccaaattcttctaagagattataaagaaaaatgccattctggtggagaagaaggagaaaattctggaccaacaacaggtttaattacaccaaaagaagaagatatagaaaaagatggcccagaaggcgccgcagaagaagaccatacagacgccctgttcgccgccgccgtagaaaacttcgaaaggtaaagagaaaaaaaaaatctttaattgttagacaatggcaaccagacagtataagaacttgtaaaattataggacagtcagctatagttgttggggctgaaggaaagcaaatgtactgttatactgtcaataagttaattaatgtgcccccaaaaacaccatatgggggaggctttggagtagaccaatacacactgaaatacttatatgaagaatacagatttgcacaaaacatttggacacaatctaatgtactgaaagacttatgcagatacataaatgttaagctaatattctacagagacaacaaaacagactttgtcctttcctatgacagaaacccaccttttcaactaacaaaatttacatacccaggagcacacccacaacaaatcatgcttcaaaaacaccacaaattcatactatcacaaatgacaaagcctaatggaagactaacaaaaaaactcaaaattaaacctcctaaacaaatgctttctaaatggttcttttcaaaacaattctgtaaataccctttactatctcttaaagcttctgcactagaccttaggcactcttacctaggctgctgtaatgaaaatccacaggtatttttttattatttaaaccatggatactacacaataacaaactggggagcacaatcctcaacagcatacagacctaactccaaggtgacagacacaacatactacagatacaaaaatgacagaaaaaatattaacattaaaagccatgaatacgaaaaaagtatatcatatgaaaacggttattttcaatctagtttcttacaaacacagtgcatatataccagtgagcgtggtgaagcctgtatagcagaaaaaccactaggaatagctatttacaatccagtaaaagacaatggagatggtaatatgatataccttgtaagcactctagcaaacacttgggaccagcctccaaaagacagtgctattttaatacaaggagtacccatatggctaggcttatttggatatttagactactgtagacaaattaaagctgacaaaacatggctagacagtcatgtactagtaattcaaagtcctgctatttttacttacccaaatccaggagcaggcaaatggtattgtccactatcacaaagttttataaatggcaatggtccgtttaatcaaccacctacactgctacaaaaagcaaagtggtttccacaaatacaataccaacaagaaattattaatagctttgtagaatcaggaccatttgttcccaaatatgcaaatcaaactgaaagcaactgggaactaaaatataaatatgtttttacatttaagtggggtggaccacaattccatgaaccagaaattgctgaccctagcaaacaagagcagtatgatgtccccgatactttctaccaaacaatacaaattgaagatccagaaggacaagaccccagatctctcatccatgattgggactacagacgaggctttattaaagaaagatctcttaaaagaatgtcaacttacttctcaactcatacagatcagcaagcaacttcagaggaagacattcccaaaaagaaaaagagaattggaccccaactcacagtcccacaacaaaaagaagaggagacactgtcatgtctcctctctctctgcaaaaaagataccttccaagaaacagagacacaagaagacctccagcagctcatcaagcagcagcaggagcagcagctcctcctcaagagaaacatcctccagctcatccacaaactaaaagagaatcaacaaatgcttcagcttcacacaggcatgttaccttaaccagatttaaacctggatttgaagagcaaacagagagagaattagcaattatatttcataggccccctagaacctacaaagaggaccttccattctatccctggctaccacctgcaccccttgtacaatttaaccttaacttcaaaggctaggccaacaatgtacacttagtaaagcatgtttattaaagcacaacccccaaaataaatgtaaaaataaaaaaaaaaaaaaaaaaataaaaaattgcaaaaattcggcgctcgcgcgcatgtgcgcctctggcgcaaatcacgcaacgctcgcgcgcccgcgtatgtctctttaccacgcacctagattggggtgcgcgcgctagcgcgcgcaccccaatgcgccccgccctcgttccgacccgcttgcgcgggtcggaccacttcgggctcgggggggcgcgcctgcggcgcttttttactaaacagactccgagccgccatttggccccctaagctccgcccccctcatgaatattcataaaggaaaccacataattagaattgccgaccacaaactgccatatgctaattagttccccttttacaaagtaaaaggggaagtgaacatagccccacacccgcaggggcaaggccccgcacccctacgtcactaaccacgcccccgccgccatcttgggtgcggcagggcgggggc

SEQ ID NO: 887 - protein, 124 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFER

SEQ ID NO: 888 - protein, 271 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFESGVDHNSMNQKLLTLANKSSMMSPILSTKQYKLKIQKDKTPDLSSMIGTTDEALLKKDLLKECQLTSQLIQISKQLQRKTFPKRKRELDPNSQSHNKKKRRHCHVSSLSAKKIPSKKQRHKKTSSSSSSSSRSSSSSSRETSSSSSTN

SEQ ID NO: 889 - protein, 267 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFERSASNFRGRHSQKEKENWTPTHSPTTKRRGDTVMSPLSLQKRYLPRNRDTRRPPAAHQAAAGAAAPPQEKHPPAHPQTKRESTNASASHRHVTLTRFKPGFEEQTERELAIIFHRPPRTYKEDLPFYPWLPPAPLVQFNLNFKG

SEQ ID NO: 890 - protein, 50 aa, Gammatorquevirus sp.
MRRRRTKYGCLKLLTLMIISAVAGIHLLTFLLPYFLLATKIVILLLTKFF

SEQ ID NO: 891 - protein, 662 aa, Gammatorquevirus sp.
MPFWWRRRRKFWTNNRFNYTKRRRYRKRWPRRRRRRRPYRRPVRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYTITNWGAQSSTAYRPNSKVTDTTYYRYKNDRKNINIKSHEYEKSISYENGYFQSSFLQTQCIYTSERGEACIAEKPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 892 - protein, 215 aa, Gammatorquevirus sp.
MPFWWRRRRKFWTNNRFNYTKRRRYRKRWPRRRRRRRPYRRPVRRRRRKLRKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 893 - protein, 129 aa, Gammatorquevirus sp.
MPFWWRRRRKFWTNNRFNYTKRRRYRKRWPRRRRRRRPYRRPVRRRRRKLRKISKQLQRKTFPKRKRELDPNSQSHNKKKRRHCHVSSLSAKKIPSKKQRHKKTSSSSSSSSRSSSSSSRETSSSSSTN

SEQ ID NO: 894-924 - skipped (31 entries)

SEQ ID NO: 925 - protein, 662 aa, Gammatorquevirus sp.; identical to SEQ ID NO: 891

SEQ ID NO: 926 - protein, 58 aa, Gammatorquevirus sp.
MPFWWRRRRKFWTNNRFNYTKRRRYRKRWPRRRRRRRPYRRPVRRRRRKLRKVKRKKK

SEQ ID NO: 927 - protein, 202 aa, Gammatorquevirus sp.
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYL

SEQ ID NO: 928 - protein, 79 aa, Gammatorquevirus sp.
NHGYYTITNWGAQSSTAYRPNSKVTDTTYYRYKNDRKNINIKSHEYEKSISYENGYFQSSFLQTQCIYTSERGEACIAE

SEQ ID NO: 929 - protein, 160 aa, Gammatorquevirus sp.
KPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFK

SEQ ID NO: 930 - protein, 163 aa, Gammatorquevirus sp.
WGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 931-948 - skipped (18 entries)

SEQ ID NO: 949 - protein, 21 aa, Artificial Sequence
  Description of Artificial Sequence: Synthetic peptide
  MOD_RES (1)..(1): W or F
  MOD_RES (2)..(8): Any amino acid
  MOD_RES (10)..(12): Any amino acid
  MOD_RES (14)..(14): Any amino acid
  MOD_RES (16)..(20): Any amino acid
XXXXXXXXHXXXCXCXXXXXH

SEQ ID NO: 950 - protein, 22 aa, Artificial Sequence
  Description of Artificial Sequence: Synthetic peptide
  MOD_RES (6)..(7): Any amino acid
  MOD_RES (9)..(9): Any amino acid
  MOD_RES (15)..(16): Any amino acid
YNCSPXXDXGASKRXXNTSVAK

SEQ ID NO: 951 - DNA, 51 bp, Artificial Sequence
  Description of Artificial Sequence: Synthetic oligonucleotide
  modified_base (45)..(45): a, c, t, g, unknown or other
aggtgagtgaaaccaccgaagtcaaggggcaattcgggctagggncagtct

SEQ ID NO: 952 - DNA, 50 bp, Alphatorquevirus sp.
aggtgagtttacacaccgcagtcaaggggcaattcgggctcgggactggc

SEQ ID NO: 953 - DNA, 50 bp, Betatorquevirus sp.
aggtgagtgaaaccaccgaagtcaaggggcaattcgggctagatcagtct

SEQ ID NO: 954 - DNA, 50 bp, Gammatorquevirus sp.
aggtgagtgaaaccaccgaggtctaggggcaattcgggctagggcagtct

SEQ ID NO: 955 - protein, 6 aa, Artificial Sequence
  Description of Artificial Sequence: Synthetic 6xHis tag
HHHHHH

SEQ ID NO: 956 - protein, 237 aa, Unknown
  Description of Unknown: Anelloviridae family sequence
RRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGY

SEQ ID NO: 957 - protein, 232 aa, Unknown
  Description of Unknown: Anelloviridae family sequence
RRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWP

SEQ ID NO: 958 - protein, 238 aa, Unknown
  Description of Unknown: Anelloviridae family sequence
RRRRRWRKRPTVRRKLKKLTIQQWQPKTIRKCCIQGLHCLFLVTEDTISRNYRMYEHSYTGEYYPGGGGFSITRYSLDGLYEQHQLDRNWWTNSNTNLPLVRYTGCKIKFYQSWSVDYICNYSLTWPMVATQLLYQSCQPSFMMMNKNSIMIPSKLTKPIKKGYKTIKEKPPHEMLNRWYFAKDLSKVGLLMLTAASASFDHYYQATDSLSNNCTFESLNPYFYMRHDFILFPVTGYV

SEQ ID NO: 959 - protein, 238 aa, Unknown
  Description of Unknown: Anelloviridae family sequence
RRRRRWVRRKPFYKRKIKRLNIVEWQPKSIRKCRIKGMLCLFQTTEDRLSYNFDMYEESIIPEKLPGGGGFSIKNISLYALYQEHIHAHNIFTHTNTDRPLARYTGCSLKFYQSKDIDYVVTYSTSLPLRSSMGMYNSMQPSIHLMQQNKLIVPSKQTQKRRKPYIKKHISPPTQMKSQWYFQHNIANIPLLMIRTTALTLDNYYIGSRQLSTNVTIHTLNTTYIQNRDWGDRNKTYY

SEQ ID NO: 960 - protein, 240 aa, Unknown
  Description of Unknown: Anelloviridae family sequence
RRPVRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYTITNWGAQSSTAYR

SEQ ID NO: 961 - protein, 240 aa, Unknown
  Description of Unknown: Anelloviridae family sequence
RFTKTRRRRKRKKVRRKLKKITIKQWQPDSVKKCKIKGYSTLVMGAQGKQYNCYTNQASDYVQPKAPQGGGFGCEVFNLKWLYQEYTAHRNIWTKTNEYTDLCRYTGAQIILYRHPDVDFIVSWDNQPPFLLNKYTYPELQPQNLLLARRKRIILSQKSNPKGKLRIKLRIPPPKQMITKWFFQRDFCDVNLFKLCASAASFRYPGISHGAQSTIFSAYALNTDFYQCSDWCQTNTETGY

SEQ ID NO: 962 - protein, 239 aa, Unknown
  Description of Unknown: Anelloviridae family sequence
GRRTYTRRAVRRRRRPRKRLVLTQWSPQTVRNCSIRGIVPMVICGHTKAGRNYAIHSEDFTTQIQPFGGSFSTTTWSLKVLWDEHQKFQNRWSYPNTQLDLARYRGVTFWFYRDQKTDYIVQWSRNPPFKLNKYSSAMYHPGMMMQAKRKLVVPSFQTRPKGKKRYRVTIKPPNMFADKWYTQEDLCPVPLVQIVVSAASLLHPFCPPQTNNPCITFQVLKDIYDECIGVNETMKDKYK

SEQ ID NO: 963 - protein, 240 aa, Unknown
  Description of Unknown: Anelloviridae family sequence
RRRRRRGRRRRRRRRRHKPTLILRQWQPDCIRHCKITGWMPLIICGKGSTQFNYITHADDITPRGASYGGNFTNMTFSLEAIYEQFLYHRNRWSASNHDLELCRYKGTTLKLYRHPEVDYIVTYSRTGPFEISHMTYLSTHPMLMLLNKHHIVVPSLKTKPRGRKAIKVRIRPPKLMNNKWYFTRDFCNIGLFQLWATGLELRNPWLRMSTLSPCIGFNVLKNSIYTNLSNLPQYKNERL

SEQ ID NO: 964 - protein, 233 aa, Unknown
  Description of Unknown: Anelloviridae family sequence
RRRRRFRRRGRKAKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSINTFNNDNSD

SEQ ID NO: 965 - protein, 233 aa, Unknown
  Description of Unknown: Anelloviridae family sequence
RRWRRKGKRSRKKKIIIRQWQPNYTRRCNIVGYMPLLICGENTVATNYATHSDDSYYPGPFGGGMTTDKFTLRILYDEYKRFMNYWTSSNEDLDLCRYLGCTLYVFRHPEVDFIIIINTSPPFLDTEITGPSIHPGMMALNKRSRWIPSIKNRPGRKHYIKIKVGAPRMFTDKWYPQTDLCDMTLLTIFASAADMQYPFGSPLTDTIVVSFQVLQSMYNDCLSVLPDNFAETS

SEQ ID NO: 966 - protein, 232 aa, Unknown
  Description of Unknown: Anelloviridae family sequence
RRWKRKGRRRRKAKIIIRQWQPNYRRRCNIVGYLPILICGGNTVSRNYATHSDDTNYPGPFGGGMTTDKFSLRILYDEYKRFMNYWTASNEDLDLCRYLGCTFYFFRHPEVDFIIKINTMPPFLDTTITAPSIHPGLMALDKRARWIPSLKNRPGKKHYIKIRVGAPKMFTDKWYPQTDLCDMTLLTIYATAADMQYPFGSPLTDTVVVNSQVLQSMYDETISILPDEKTKR

SEQ ID NO: 967 - protein, 170 aa, Beak and feather disease virus
RRRRRRFSTNRIYTLRLTRQFQFKINKQLIFNADYITFALDDFLQAVPNPHTLNFEDYRIKLAKMEMRPTGGHYTGFGHTAVIQDSRITRFKTTADPLAPFDGAKKWFVSRGFKRLLRPKPQITIEGPNSAGTKVRHYGIAFSFPQPEQTVTKLTLYVQFRQFAPNNPST

SEQ ID NO: 968 - protein, 179 aa, Orthohepevirus A
GAILRRQYNLSTSPLTSSVASGTNLVLYAAPLNPLLPLQDGTNTHIMATEASNYAQYRVVRATIRYRPLVPNAVSISFWPQTTTTPTSVDMNSITSTDVRILVQPGIASELVIPSERLHYRTRTGVAEEEATSGLVMLCIHGSPVNSALGLLDFALELEFRNLTPGNTNTRVSRYTSTA